SEQUENCE LISTING (195 sequences)

SEQ ID NO: 1 - DNA, 6015 bp, Homo sapiens
ctctctctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagagatgtacggggaccctctatgcaccgaacccaatacgttcattccccgtatgatcgtcctggttggaaccctcggttctgcatcatctcggggaaccagctgctcatgctggatgaggatgagatacaccccctactgatccgggaccggaggagcgagtccagtcgcaacaaactgctgagacgcacagtctccgtgccggtggaggggcggccccacggcgagcatgaataccacttgggtcgctcgaggaggaagagtgtcccaggggggaagcagtacagcatggagggtgcccctgctgcgcccttccggccctcgcaaggcttcctgagccgacggctaaaaagctccatcaaacgaacgaagtcacaacccaaacttgaccggaccagcagctttcgccagatcctgcctcgcttccgaagtgctgaccatgaccgggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtaacaacttcatcaggaacaaaatgctttgcctgtcggtctgcggccgaaagagacaaatggattgagaatctgcagcgggcagtaaagcccaacaaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggacttcctttcagacatggccatgtctgaggtagaccggttcatggaacgggagcacctcatattccgcgagaacacgcttgccactaaagccatagaagagtatatgagactgattggtcagaaatacctcaaggatgccattggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactgcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacagctccatcgaccttcagtccttcatggctcgaggcctcaacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggtgaaggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcaggagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttaggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttctttta

SEQ ID NO: 2 - DNA, 5966 bp, Homo sapiens
ctctctctcggctgccgctgctgccgttggctcttattctcctcctcctcctcctctctcctcctctctgcttctctctgctcctctctcctcctctctcctcctcctcctcctccacctcctcctccttctccccctctttctccccctctttctctcttctttctcccccgtccccccgccccctccccccaggcctgatgagcaggtctcgagcctccatccatcgggggagcatccccgcgatgtcctatgcccccttcagagatgtacggggaccctctatgcaccgaacccaatacgttcattccccgtatgatcgtcctggttggaaccctcggttctgcatcatctcggggaaccagctgctcatgctggatgaggatgagatacaccccctactgatccgggaccggaggagcgagtccagtcgcaacaaactgctgagacgcacagtctccgtgccggtggaggggcggccccacggcgagcatgaataccacttgggtcgctcgaggaggaagagtgtcccaggggggaagcagtacagcatggagggtgcccctgctgcgcccttccggccctcgcaaggcttcctgagccgacggctaaaaagctccatcaaacgaacgaagtcacaacccaaacttgaccggaccagcagctttcgccagatcctgcctcgcttccgaagtgctgaccatgaccgggcccggctgatgcaaagctttaaggagtcacactctcatgagtccttgctgagtcctagcagtgcagctgaggcattggagctcaacttggatgaagattccattatcaagccagtgcacagctccatcctgggccaggagttctgttttgaggtaacaacttcatcaggaacaaaatgctttgcctgtcggtctgcggccgaaagagacaaatggattgagaatctgcagcgggcagtaaagcccaacaaggacaacagccgccgggtagacaatgtgctaaagctgtggatcatagaggcccgggagctgccccccaagaagcggtactactgtgagctctgcctggatgacatgctgtatgcacgcaccacctccaagccccgctctgcctctggggacaccgtcttctggggcgagcacttcgagtttaacaacctgccggctgtccgtgccctgcggctgcatctgtaccgtgactcagacaaaaagcgcaagaaggacaaggcaggctatgtcggcctggtgactgtgccagtggccaccctggctgggcgccacttcacagagcagtggtaccctgtaaccctgccaacaggcagtgggggatctgggggcatgggttcgggagggggagggggctcggggggtggctcagggggcaagggcaaaggaggttgcccggctgtgcggctgaaagcacgttaccagacaatgagcatcttgcccatggagctatataaagagtttgcagagtatgtcaccaaccattatcggatgctgtgtgcagtcttggagcccgccctgaatgtcaaaggcaaggaggaggttgccagtgcactagttcacatcctgcagagtacaggcaaggccaaggacttcctttcagacatggccatgtctgaggtagaccggttcatggaacgggagcacctcatattccgcgagaacacgcttgccactaaagccatagaagagtatatgagactgattggtcagaaatacctcaaggatgccattggagaattcatccgtgctctgtatgaatctgaggaaaactgcgaggtagaccctatcaagtgcacagcatccagtttggcagagcaccaggccaacctgcgaatgtgctgtgagttggccctgtgcaaggtggtcaactcccactgcgtgttcccgagggagctgaaggaggtgtttgcttcgtggcggctgcgctgcgcagagcgaggccgggaggacatcgcagacaggcttatcagcgcctcactcttcctgcgcttcctctgcccagcgattatgtcgcccagtctctttgggcttatgcaggagtacccagatgagcagacctcacgaaccctcaccctcattgccaaggtcatccagaacctggccaacttttccaagtttacctcaaaggaggactttctgggcttcatgaatgagtttctggagctggaatggggttccatgcagcagtttttgtatgagatctccaatctggacacgctaaccaacagcagtagctttgagggttacatcgacttgggccgagagctctccacactgcatgccctactctgggaggtgctgccccagctcagcaaggaagccctcctgaagctgggtccactgccccggctcctcaacgacatcagcacagctctgaggaaccccaacatccaaaggcagccaagccgccagagtgagcggccccggcctcagcctgtggtactgcgggggccatcggctgagatgcagggctacatgatgcgggacctcaacagctctatggacatggctcgcctcccctccccaaccaaggaaaagccacccccaccaccgcctggtggtggtaaagacctgttctatgtaagccgtccacccctggcccgttcctcaccagcatactgcacgagcagctcggacatcacagagccagagcagaagatgctgagtgtcaacaagagtgtgtccatgctggacttacagggtgatgggcctggtggccgcctcaacagcagcagtgtttcgaacctggcggccgtaggggacctgctgcactcaagccaggcctcgctgacagcagccttggggctacggcctgcgcctgccggacgcctctcccaggggagtggctcatccatcacggcggctggcatgcgcctcagccagatgggtgtcaccacagacggtgtccctgcccagcaactgcgaatccccctctccttccagaaccctctcttccacatggctgctgatgggccaggtcccccaggcggccatggagggggcggtggccatggcccaccttcctcccatcaccaccaccaccaccatcaccaccaccgaggtggagagccccctggggacacctttgccccattccatggctatagcaagagtgaggacctctcttccggggtccccaagccccctgctgcctccatccttcatagccacagctacagtgatgagtttggaccctctggcactgacttcacccgtcggcagctttcactccaggacaacctgcagcacatgctgtcccctccccagatcaccattggtccccagaggccagccccctcagggcctggaggtgggagcggtgggggcagcggtgggggtggcgggggccagccgcctccattgcagaggggcaagtctcagcagttgacagtcagcgcagcccagaaaccccggccatccagcgggaatctattgcagtccccagagccaagttatggccccgcccgtccacggcaacagagcctcagcaaggagggcagcattgggggcagcgggggcagcggtggcggagggggtggggggctgaagccctccatcaccaagcagcattctcagacaccatccacattgaaccccacaatgccagcctctgagcggacagtggcctgggtctccaacatgcctcacctgtcggctgacatcgagagtgcccacatcgagcgggaagagtacaagctcaaggagtactcaaaatcgatggatgagagccggctggatagggagtacgaggaggagattcactcactgaaagagcggctgcacatgtccaaccggaagctggaagagtatgagcggaggctgctgtcccaggaagaacaaaccagcaaaatcctgatgcagtatcaggcccgactggagcagagtgagaagaggctaaggcagcagcaggcagagaaggattcccagatcaagagcatcattggcaggctgatgctggtggaggaggagctgcgccgggaccaccccgccatggctgagccgctgccagaacccaagaagaggctgctcgacgctcagagaggcagcttccccccttgggtccaacaaacccgcgtgtgacgctggccccaccgtggaatggcctggcccccccagccccaccacccccaccccggctgcagattacggagaacggcgagttccgaaacaccgcagaccactagcccacccagcatcagagaccttctcttcctttcctgtgcaccccaccctgtaacagcaccaaccaccaggattggacatcaccgaggaacagcgggattgcctccccgaatgcctccctgggaggcacactgattgcccacccccaccactgcaccatttccaggagggagagtggggaccctcagccgcccccttttccttcccattggggtgctgccctctctttgacccccagggacccttgccccaggacaccgcctaccccgtacagaccccttcactccggggtgctatccccatcctctgcctcatcgttcccctgagcactgggggacagaccctcacccccaccctgggggtgtggcacctccaaactttcaacttcagggtgatttttttagcagtaaccagagctgacaatctaactcccctccaccgccccattttggcctcccctgccccccttgttatggggaggggaccccgggtgagggggccctattaccccttgatttctcaggagcgtctgggggggctcagcacgcacaaactccttctccttctaccactcttaaatttactccctccccacccagaacccagatggggtggagggggccaccggggcagggagggggcggcaaggggggaatgggagttgtctccccttctccccacacctgatctgctctcggctggtcccagagcggggtgagggggcttatgcccccccctcccccagtgtgttgggtggggtggaattgaggttagggtgaggggtcagggtttaggagggtgtgtatgttgggaggacaggctagttgatctgtcctactctgacacacagtcccctctgccccttccttctctcttcttggtctctactcccagggggaggggggaacttactctaggaaaagccatgtctctctcccccagggtggggggacctgtgttggaggaggggtgttggggggcccccttccatgactctgtcccctgggggaggtaggacagggctgggcttccctctcatcctccccctcccaatctccttccacctccctccctcccgccagctccacgatttttcggtgtttctctgtacatagttttctggcgggataggggaggtaggatggatggggtttggggtgggtaggccatgggaggggagaagcccctccttggcaccccctcttccctgactgctgtcccctacccagccttgcccccttcatccttttgcgtttggtattgagactctcctagactctactcctctttcttttgtatggacagttccccttcagtcccatccccctacacatacacccagccggggccaaatttatacttatataaaagttgtaaatatgtgaaattttatccctgtgccctttccccacctcaggccctacccctggaccctccccaaccttccttctctcttctttggctgttgtaattatctggggtttgtactgtacatatccggggtgtgtgtgtgtgggctgggggcaacccttctgtacagagcttcctggccccctccccccccgcccctctgcttccctccccacccaccacctcaagggtagggagttgctcttcctacctgttttattttgttttctcgttctccctccccaccccactcccagccttatctatcccccctcactgtccccttttctccactcccagccccatttcctttttttctggagtgtgtggtgaaacagaaaaaaacatgtttaataaacggagattgttctttta

SEQ ID NO: 3 - protein, 1343 aa, Homo sapiens [sequence omitted]

SEQ ID NO: 4 - protein, 1292 aa, Homo sapiens
MSRSRASIHRGSIPAMSYAPFRDVRGPSMHRTQYVHSPYDRPGWNPRFCIISGNQLLMLDEDEIHPLLIRDRRSESSRNKLLRRTVSVPVEGRPHGEHEYHLGRSRRKSVPGGKQYSMEGAPAAPFRPSQGFLSRRLKSSIKRTKSQPKLDRTSSFRQILPRFRSADHDRARLMQSFKESHSHESLLSPSSAAEALELNLDEDSIIKPVHSSILGQEFCFEVTTSSGTKCFACRSAAERDKWIENLQRAVKPNKDNSRRVDNVLKLWIIEARELPPKKRYYCELCLDDMLYARTTSKPRSASGDTVFWGEHFEFNNLPAVRALRLHLYRDSDKKRKKDKAGYVGLVTVPVATLAGRHFTEQWYPVTLPTGSGGSGGMGSGGGGGSGGGSGGKGKGGCPAVRLKARYQTMSILPMELYKEFAEYVTNHYRMLCAVLEPALNVKGKEEVASALVHILQSTGKAKDFLSDMAMSEVDRFMEREHLIFRENTLATKAIEEYMRLIGQKYLKDAIGEFIRALYESEENCEVDPIKCTASSLAEHQANLRMCCELALCKVVNSHCVFPRELKEVFASWRLRCAERGREDIADRLISASLFLRFLCPAIMSPSLFGLMQEYPDEQTSRTLTLIAKVIQNLANFSKFTSKEDFLGFMNEFLELEWGSMQQFLYEISNLDTLTNSSSFEGYIDLGRELSTLHALLWEVLPQLSKEALLKLGPLPRLLNDISTALRNPNIQRQPSRQSERPRPQPVVLRGPSAEMQGYMMRDLNSSMDMARLPSPTKEKPPPPPPGGGKDLFYVSRPPLARSSPAYCTSSSDITEPEQKMLSVNKSVSMLDLQGDGPGGRLNSSSVSNLAAVGDLLHSSQASLTAALGLRPAPAGRLSQGSGSSITAAGMRLSQMGVTTDGVPAQQLRIPLSFQNPLFHMAADGPGPPGGHGGGGGHGPPSSHHHHHHHHHHRGGEPPGDTFAPFHGYSKSEDLSSGVPKPPAASILHSHSYSDEFGPSGTDFTRRQLSLQDNLQHMLSPPQITIGPQRPAPSGPGGGSGGGSGGGGGGQPPPLQRGKSQQLTVSAAQKPRPSSGNLLQSPEPSYGPARPRQQSLSKEGSIGGSGGSGGGGGGGLKPSITKQHSQTPSTLNPTMPASERTVAWVSNMPHLSADIESAHIEREEYKLKEYSKSMDESRLDREYEEEIHSLKERLHMSNRKLEEYERRLLSQEEQTSKILMQYQARLEQSEKRLRQQQAEKDSQIKSIIGRLMLVEEELRRDHPAMAEPLPEPKKRLLDAQRGSFPPWVQQTRV

SEQ ID NO: 5 - DNA, 2345 bp, Homo sapiens
gtacaggggctggagcatgtgggatgagattgatgtaatgtagggtctcctgtgtgagatgcagagggagggggttatctgtgtgcaaaggttgaaggattcaactcaagttggttgggggatgtcatggcacaggggacagaacagaaaagaactagaatagggatctgtgagcagcaggagaggggtagggtggcagagagaagacagacagacaggctggaaagggaatgaaggtgaagccaaggagggactcctcagggactcctcaggccaagaaggatgggctctagcccaggatcaaaggagctgtacaggaggagagtgaccctggaggaatgtttaaggaatgcagggaaggggttggtaggtgagtgagcaataggctgtaggtggaagggtgtcagggaaggtcaggaaatacaggggcagcaggttggagtggggctgggggtggctgaatgaatggatgatggctagggctcaaggacctcatcagtgagggaagagacagtatagagcatggcagagaaggggaggctgggacaggtgtgcagggtgacagaatgggaagcaacccatggactgaggcatgaagaagcagccagcggagaagtccagaaggcactgtccctgagaccaggctgaaggagacctccactgtttgcctttgttgcctgccatttggggttcctctctgggtttccccctcacccagtcactccccagggagaaccatgccctccctttcccccatgtctggccacccccaggattgggcaggtagggaggttgggataaagtgagtcacacctttccctgcccccctcccatgttgccagagctggatttggggccggcagggggtgagggcatggtattcctggccgcgggggcgggggggggggtccgggggccgggggagcgtcgcgctgacggcagccagagcctgcgatgacggggctgctataaataacttcttggaggctcccacacccaagctcccctcccgctttcccactgctctctactcttcatcccctgcccatctccataccgcttttgtattgctatcctacccctcattattccatgcccctagccccctttatcttctgccctcctgcagtgatttttttgcattccatcccctcttagccctcacctcggttctcccggccatctctccagttggccttcctcctcttctcctgtcctctgtcttgctgcacatacctttgtctccccctttcttcttcttgccctacctcctcttcttccctagtccgtgtattctgtcttttatcctctttgagctcttttctgcccacagctttctcctatttcttatgcttttccctcactctttcccctgcttctgctaaaacttgtcctcttatgctgtgttcattcattctttgaatcattaaatgtttatcaggcactagccgtgtgccaggcccaggctagacatatctcttctctgtgccttcacttctttacttccactttttcctttatactgaggctctggtttctggggttacctggaggtactacctagaagtgccccaggcccactttgttctctccttttttttttttcttttctgccatggtccatttctgggttgagatatttctagatgtccccagtcctcgcaatcccttaggtgtgagatggtgggagtttcttttttttcctttttttttttttaaatagaaatagggtctcactgtgttgcccagactggtcttgaactcctgggctcaagtgaccctcccacctcggcctttgaaatgttgggattacaggtgtgagccaccaggcccaggtggagcaggggagttccttaaaggattctgatttttctcacatccctcacgtccttcctgataggcagggtttctttctgtgtctgtttgggaagggtgttcagggggccttctctccaagtctccatcctggaacagactgatgatgcagggtacctatgtgtctaagaagagtaggggggccgggcgcggtggctcatgcctgtaatcccagcactttgggaggctgaatcacttgaggtcaggagtttgagaccagcctgaccaacagggtgaaaccccgtctcagctaaaaatacaaaaaaaaaaaagaaaaaaaaattagctgggtgtgctgaggcaggagagacgcttgagcccaggaggcagaagttgcagcaagccgagatcacaccactgtactccagcctgggcgacagagcaagactgtctcaaaaaaaaaaaaaaaaaaaaaaaaggaagagtgggaagccctgatcccttcctctcctgaacctcctgcctgccag

SEQ ID NO: 6 - DNA, 127 bp, Homo sapiens
gtgagtgttgtgccctcagggaaaggtgacttgggaatgggcacttgcttgggggttagtgaggacagggcaaattcacgagattgggttgtgcagaggctgacacttggattttcctgggcctcag

SEQ ID NO: 7 - DNA, 211 bp, Homo sapiens
gtatggcccacactcaggccctcttcttcccaaacctgccagatgtccaccccagaccccaagtccacccttccacagcttgatacttcctaacccagagtcctaggactccagcctccaacacctgattctgaaatttccccaaccctggccacccccttccctgcccttggaaagtgtgaccacaccctcttgtgcccccaccccccag

SEQ ID NO: 8 - DNA, 207 bp, Homo sapiens
gtgagggaagcttcaggagtgggcagggcagggagtggcagggcagggagtggcagggctgggggtcggcaagaagggtctcctgagtccccagagatcctgagatggggaggctatgataccttgtgtgtgtgtgtatgtgtgtgtatgtgtgtgtgtgtgtgtgtgtgtgtgtatgtgacctttatcttctgcattcttggctag

SEQ ID NO: 9 - DNA, 206 bp, Homo sapiens
gtcagcagatcccctctttgccctatccccagatggctccagaggttcctggagcctgagaaactaccctttgaagattttttttctccccttgtttctcgaggtgtcaccactactatcccaactcaggccccctccacctgcaccctcagaggccctcttagagctgggcactgagcccccaggtaacagcctcacccttccag

SEQ ID NO: 10 - DNA, 693 bp, Homo sapiens
gtgagcaccctgggacagccaggcctgtgccctaggagcccttctcctattctagatactcctcactgggccccacatgcatctctctagggcttgaaagaagggaggaaaaagcaccaagttctcaggggagacgataaggagacaggtacagtcagtggtaggctgagagccctttacagcctgagggagtgagagatttggagctctaggaatagggctgaggctccaccaactcacggcttagttgtaagcctagagcatccctgctgcaagctctgatttgctgtccctctgcctgcccatgctagtccccaggctgaggttcagccagcatgtcatgtcagccatgtgtcaaaatgttcaaacatctcagtaatagctagtgaataagcacttcccccagcccccgaccacaaccccacagacctccccatgatccagcacttagagcagtgacagcagaagcctagcagggcctgcagcctgctccagagtcccagcctccattctgataggtggtgcccgtgcttcatttgctgctcattattttgatgatggccctgctttttcctctgcccgtgtcttcctccatgacaccatacccatcccaccattcccgcctctcctttcatttgtccacatctctctccttctctgtctgtgctcgcccctctttccatctctctccag

SEQ ID NO: 11 - DNA, 23 bp, Homo sapiens
ctatgcccccttcagagatgtac

SEQ ID NO: 12 - DNA, 18 bp, Homo sapiens
tggttccccgagatgatg

SEQ ID NO: 13 - DNA, 20 bp, Homo sapiens
ctccacctcctcctccttct

SEQ ID NO: 14 - DNA, 20 bp, Homo sapiens
cccaccacgtacctctgaag

SEQ ID NO: 15 - DNA, 25 bp, Homo sapiens
tcatgctggatgagtatgagataca

SEQ ID NO: 16 - DNA, 19 bp, Homo sapiens
cccctgggacactcttcct

SEQ ID NO: 17 - DNA, 20 bp, Homo sapiens
ctctatgcaccgaacccaat

SEQ ID NO: 18 - DNA, 20 bp, Homo sapiens
gaggactctccccattctcc

SEQ ID NO: 19 - DNA, 20 bp, Homo sapiens
tcgcaacaaactgctgagac

SEQ ID NO: 20 - DNA, 20 bp, Homo sapiens
actttgtctccgccttctcc

SEQ ID NO: 21 - DNA, 18 bp, Homo sapiens
ctcgcaaggcttcctgag

SEQ ID NO: 22 - DNA, 19 bp, Homo sapiens
cggtcatggtcagcacttc

SEQ ID NO: 23 - DNA, 20 bp, Homo sapiens
gctctaggaggaagagtgtc

SEQ ID NO: 24 - DNA, 20 bp, Homo sapiens
tgccctctttctcagactcc

SEQ ID NO: 25 - DNA, 17 bp, Homo sapiens
gaccgtgctcggctgat

SEQ ID NO: 26 - DNA, 20 bp, Homo sapiens
tgcactggcttgataatgga

SEQ ID NO: 27 - DNA, 20 bp, Homo sapiens
acggctaaaaagctccatca

SEQ ID NO: 28 - DNA, 20 bp, Homo sapiens
ctccctctgcatctcacaca

SEQ ID NO: 29 - DNA, 22 bp, Homo sapiens
ggagtgctgtgttgaggtaaca

SEQ ID NO: 30 - DNA, 18 bp, Homo sapiens
tactgcccgctccagatt

SEQ ID NO: 31 - DNA, 20 bp, Homo sapiens
gcccaggagttctgttttga

SEQ ID NO: 32 - DNA, 20 bp, Homo sapiens
cctaccctttcctccagtcc

SEQ ID NO: 33 - DNA, 21 bp, Homo sapiens
taaagcccaacaaggacaaca

SEQ ID NO: 34 - DNA, 20 bp, Homo sapiens
agaagacggtgtccccagag

SEQ ID NO: 35 - DNA, 20 bp, Homo sapiens
gccgaaagagacaaatggat

SEQ ID NO: 36 - DNA, 20 bp, Homo sapiens
cccaagcctctcctccttta

SEQ ID NO: 37 - DNA, 21 bp, Homo sapiens
gccaagcacttcctttcagac

SEQ ID NO: 38 - DNA, 21 bp, Homo sapiens
caatggcatccttgaggtatt

SEQ ID NO: 39 - DNA, 20 bp, Homo sapiens
ccattatcggatgctgtgtg

SEQ ID NO: 40 - DNA, 20 bp, Homo sapiens
tgtcctcactaacccccaag

SEQ ID NO: 41 - DNA, 20 bp, Homo sapiens
cattggagagttcatccgtg

SEQ ID NO: 42 - DNA, 20 bp, Homo sapiens
cagtgggagttgaccacctt

SEQ ID NO: 43 - DNA, 20 bp, Homo sapiens
aacacgcttgccactaaagc

SEQ ID NO: 44 - DNA, 20 bp, Homo sapiens
tgttggaggctggagtccta

SEQ ID NO: 45 - DNA, 20 bp, Homo sapiens
tcaactcccactgcgtgttc

SEQ ID NO: 46 - DNA, 20 bp, Homo sapiens
tcgtgaggtctgctcatctg

SEQ ID NO: 47 - DNA, 20 bp, Homo sapiens
ctgcgaatgtgctgtgagtt

SEQ ID NO: 48 - DNA, 20 bp, Homo sapiens
aatttgtccccattctggtg

SEQ ID NO: 49 - DNA, 23 bp, Homo sapiens
cttttccaagtttacctcaaagg

SEQ ID NO: 50 - DNA, 21 bp, Homo sapiens
ccaagtcgatgtaaccctcaa

SEQ ID NO: 51 - DNA, 20 bp, Homo sapiens
atgagcagacctcacgaacc

SEQ ID NO: 52 - DNA, 20 bp, Homo sapiens
tcatagcctccccatctcag

SEQ ID NO: 53 - DNA, 20 bp, Homo sapiens
agcaaggaatccctcctgaa

SEQ ID NO: 54 - DNA, 19 bp, Homo sapiens
tgtagccctgcatctcagc

SEQ ID NO: 55 - DNA, 20 bp, Homo sapiens
gacacgctaaccaacagcag

SEQ ID NO: 56 - DNA, 20 bp, Homo sapiens
tcgagaaacaaggggagaaa

SEQ ID NO: 57 - DNA, 20 bp, Homo sapiens
cctgaacgacatcagcacag

SEQ ID NO: 58 - DNA, 20 bp, Homo sapiens
tcgatggagctgttgaggtc

SEQ ID NO: 59 - DNA, 20 bp, Homo sapiens
tgagatgcacggctacatga

SEQ ID NO: 60 - DNA, 20 bp, Homo sapiens
tcgtctcccctgagaacttg

SEQ ID NO: 61 - DNA, 21 bp, Homo sapiens
tcaacagctctatggacatgg

SEQ ID NO: 62 - DNA, 20 bp, Homo sapiens
gcatcttctgctctggctct

SEQ ID NO: 63 - DNA, 20 bp, Homo sapiens
tgcatcgaccttcagtcctt

SEQ ID NO: 64 - DNA, 20 bp, Homo sapiens
gggcacatatggaggagatg

SEQ ID NO: 65 - DNA, 21 bp, Homo sapiens
accaagcagcattctcagaca

SEQ ID NO: 66 - DNA, 20 bp, Homo sapiens
ggctctcatccatccatttt

SEQ ID NO: 67 - DNA, 20 bp, Homo sapiens
cagtccccagagccaagtta

SEQ ID NO: 68 - DNA, 20 bp, Homo sapiens
tcttccctccctgttgtgac

SEQ ID NO: 69 - DNA, 22 bp, Homo sapiens
gctggatagggtgaaggagtac

SEQ ID NO: 70 - DNA, 19 bp, Homo sapiens
ctccagtcgggcctgatac

SEQ ID NO: 71 - DNA, 20 bp, Homo sapiens
atcgagcgggaagagtacaa

SEQ ID NO: 72 - DNA, 20 bp, Homo sapiens
aactgaccctggaggtttcc

SEQ ID NO: 73 - DNA, 21 bp, Homo sapiens
atagaatcattggcaggctga

SEQ ID NO: 74 - DNA, 18 bp, Homo sapiens
cagagcgtcgagcatcct

SEQ ID NO: 75 - DNA, 20 bp, Homo sapiens
gtcccaggaagaacaaacca

SEQ ID NO: 76 - DNA, 20 bp, Homo sapiens
ctcaggctctccctcacaac

SEQ ID NO: 77 - DNA, 21 bp, Homo sapiens
actctcaggagaggcagcttc

SEQ ID NO: 78 - DNA, 20 bp, Homo sapiens
ttcctcggtgatgtccaatc

SEQ ID NO: 79 - DNA, 20 bp, Homo sapiens
ctgccagaacccaagaagag

SEQ ID NO: 80 - DNA, 20 bp, Homo sapiens
agtgacaaaggcacagacga

SEQ ID NO: 81 - DNA, 21 bp, Homo sapiens
ccattatcggatgctgtgtgc

SEQ ID NO: 82 - DNA, 20 bp, Homo sapiens
gctttagtggcaagcgtgtt

SEQ ID NO: 83 - DNA, 18 bp, Homo sapiens
tgagggcacaacactcac

SEQ ID NO: 84 - DNA, 18 bp, Homo sapiens
ctgagggcacaacactca

SEQ ID NO: 85 - DNA, 18 bp, Homo sapiens
cctgagggcacaacactc

SEQ ID NO: 86 - DNA, 18 bp, Homo sapiens
ccctgagggcacaacact

SEQ ID NO: 87 - DNA, 18 bp, Homo sapiens
tccctgagggcacaacac

SEQ ID NO: 88 - DNA, 18 bp, Homo sapiens
ttccctgagggcacaaca

SEQ ID NO: 89 - DNA, 18 bp, Homo sapiens
tttccctgagggcacaac

SEQ ID NO: 90 - DNA, 18 bp, Homo sapiens
ctttccctgagggcacaa

SEQ ID NO: 91 - DNA, 18 bp, Homo sapiens
cctttccctgagggcaca

SEQ ID NO: 92 - DNA, 18 bp, Homo sapiens
acctttccctgagggcac

SEQ ID NO: 93 - DNA, 18 bp, Homo sapiens
cacctttccctgagggca

SEQ ID NO: 94 - DNA, 18 bp, Homo sapiens
tcacctttccctgagggc

SEQ ID NO: 95 - DNA, 18 bp, Homo sapiens
gtcacctttccctgaggg

SEQ ID NO: 96 - DNA, 18 bp, Homo sapiens
agtcacctttccctgagg

SEQ ID NO: 97 - DNA, 18 bp, Homo sapiens
aagtcacctttccctgag

SEQ ID NO: 98 - DNA, 18 bp, Homo sapiens
ccaagtcacctttccctg

SEQ ID NO: 99 - DNA, 18 bp, Homo sapiens
cccaagtcacctttccct

SEQ ID NO: 100 - DNA, 18 bp, Homo sapiens
attcccaagtcacctttc

SEQ ID NO: 101 - DNA, 18 bp, Homo sapiens
ccattcccaagtcacctt

SEQ ID NO: 102 - DNA, 18 bp, Homo sapiens
gcccattcccaagtcacc

SEQ ID NO: 103 - DNA, 18 bp, Homo sapiens
tgcccattcccaagtcac

SEQ ID NO: 104 - DNA, 18 bp, Homo sapiens
gtgcccattcccaagtca

SEQ ID NO: 105 - DNA, 18 bp, Homo sapiens
agtgcccattcccaagtc

SEQ ID NO: 106 - DNA, 18 bp, Homo sapiens
aagtgcccattcccaagt

SEQ ID NO: 107 - DNA, 18 bp, Homo sapiens
caagtgcccattcccaag

SEQ ID NO: 108 - DNA, 18 bp, Homo sapiens
gcaagtgcccattcccaa

SEQ ID NO: 109 - DNA, 18 bp, Homo sapiens
agcaagtgcccattccca

SEQ ID NO: 110 - DNA, 18 bp, Homo sapiens
aagcaagtgcccattccc

SEQ ID NO: 111 - DNA, 18 bp, Homo sapiens
ccccaagcaagtgcccat

SEQ ID NO: 112 - DNA, 18 bp, Homo sapiens
acccccaagcaagtgccc

SEQ ID NO: 113 - DNA, 18 bp, Homo sapiens
taacccccaagcaagtgc

SEQ ID NO: 114 - DNA, 18 bp, Homo sapiens
actaacccccaagcaagt

SEQ ID NO: 115 - DNA, 18 bp, Homo sapiens
tcactaacccccaagcaa

SEQ ID NO: 116 - DNA, 18 bp, Homo sapiens
cctcactaacccccaagc

SEQ ID NO: 117 - DNA, 18 bp, Homo sapiens
gtcctcactaacccccaa

SEQ ID NO: 118 - DNA, 18 bp, Homo sapiens
ctgtcctcactaaccccc

SEQ ID NO: 119 - DNA, 18 bp, Homo sapiens
ccctgtcctcactaaccc

SEQ ID NO: 120 - DNA, 18 bp, Homo sapiens
tgccctgtcctcactaac

SEQ ID NO: 121 - DNA, 18 bp, Homo sapiens
aatttgccctgtcctcac

SEQ ID NO: 122 - DNA, 18 bp, Homo sapiens
cgtgaatttgccctgtcc

SEQ ID NO: 123 - DNA, 18 bp, Homo sapiens
ctcgtgaatttgccctgt

SEQ ID NO: 124 - DNA, 18 bp, Homo sapiens
atctcgtgaatttgccct

SEQ ID NO: 125 - DNA, 18 bp, Homo sapiens
caatctcgtgaatttgcc

SEQ ID NO: 126 - DNA, 18 bp, Homo sapiens
cccaatctcgtgaatttg

SEQ ID NO: 127 - DNA, 18 bp, Homo sapiens
aacccaatctcgtgaatt

SEQ ID NO: 128 - DNA, 18 bp, Homo sapiens
acaacccaatctcgtgaa

SEQ ID NO: 129 - DNA, 18 bp, Homo sapiens
gcacaacccaatctcgtg

SEQ ID NO: 130 - DNA, 18 bp, Homo sapiens
ctgcacaacccaatctcg

SEQ ID NO: 131 - DNA, 18 bp, Homo sapiens
ctctgcacaacccaatct

SEQ ID NO: 132 - DNA, 18 bp, Homo sapiens
gcctctgcacaacccaat

SEQ ID NO: 133 - DNA, 18 bp, Homo sapiens
cagcctctgcacaaccca

SEQ ID NO: 134 - DNA, 18 bp, Homo sapiens
gtcagcctctgcacaacc

SEQ ID NO: 135 - DNA, 18 bp, Homo sapiens
gtgtcagcctctgcacaa

SEQ ID NO: 136 - DNA, 18 bp, Homo sapiens
aagtgtcagcctctgcac

SEQ ID NO: 137 - DNA, 18 bp, Homo sapiens
atccaagtgtcagcctct

SEQ ID NO: 138 - DNA, 18 bp, Homo sapiens
aaatccaagtgtcagcct

SEQ ID NO: 139 - DNA, 18 bp, Homo sapiens
gaaaatccaagtgtcagc

SEQ ID NO: 140 - DNA, 18 bp, Homo sapiens
ccaggaaaatccaagtgt

SEQ ID NO: 141 - DNA, 18 bp, Homo sapiens
gcccaggaaaatccaagt

SEQ ID NO: 142 - DNA, 18 bp, Homo sapiens
aggcccaggaaaatccaa

SEQ ID NO: 143 - DNA, 18 bp, Homo sapiens
tgaggcccaggaaaatcc

SEQ ID NO: 144 - DNA, 18 bp, Homo sapiens
agggcctgagtgtgggcc

SEQ ID NO: 145 - DNA, 18 bp, Homo sapiens
gagggcctgagtgtgggc

SEQ ID NO: 146 - DNA, 18 bp, Homo sapiens
aagagggcctgagtgtgg

SEQ ID NO: 147 - DNA, 18 bp, Homo sapiens
gaagagggcctgagtgtg

SEQ ID NO: 148 - DNA, 18 bp, Homo sapiens
agaagagggcctgagtgt

SEQ ID NO: 149 - DNA, 18 bp, Homo sapiens
aagaagagggcctgagtg

SEQ ID NO: 150 - DNA, 18 bp, Homo sapiens
gaagaagagggcctgagt

SEQ ID NO: 151 - DNA, 18 bp, Homo sapiens
ggaagaagagggcctgag

SEQ ID NO: 152 - DNA, 18 bp, Homo sapiens
gggaagaagagggcctga

SEQ ID NO: 153 - DNA, 18 bp, Homo sapiens
tgggaagaagagggcctg

SEQ ID NO: 154 - DNA, 18 bp, Homo sapiens
ttgggaagaagagggcct

SEQ ID NO: 155 - DNA, 18 bp, Homo sapiens
tttgggaagaagagggcc

SEQ ID NO: 156 - DNA, 18 bp, Homo sapiens
gtttgggaagaagagggc

SEQ ID NO: 157 - DNA, 18 bp, Homo sapiens
ggtttgggaagaagaggg

SEQ ID NO: 158 - DNA, 18 bp, Homo sapiens
aggtttgggaagaagagg

SEQ ID NO: 159 - DNA, 18 bp, Homo sapiens
caggtttgggaagaagag

SEQ ID NO: 160 - DNA, 18 bp, Homo sapiens
gcaggtttgggaagaaga

SEQ ID NO: 161 - DNA, 18 bp, Homo sapiens
ggcaggtttgggaagaag

SEQ ID NO: 162 - DNA, 18 bp, Homo sapiens
tggcaggtttgggaagaa

SEQ ID NO: 163 - DNA, 18 bp, Homo sapiens
ctggcaggtttgggaaga

SEQ ID NO: 164 - DNA, 18 bp, Homo sapiens
tctggcaggtttgggaag

SEQ ID NO: 165 - DNA, 18 bp, Homo sapiens
atctggcaggtttgggaa

SEQ ID NO: 166 - DNA, 18 bp, Homo sapiens
catctggcaggtttggga

SEQ ID NO: 167 - DNA, 18 bp, Homo sapiens
acatctggcaggtttggg

SEQ ID NO: 168 - DNA, 18 bp, Homo sapiens
aggactctgggttaggaa

SEQ ID NO: 169 - DNA, 18 bp, Homo sapiens
taggactctgggttagga

SEQ ID NO: 170 - DNA, 18 bp, Homo sapiens
ctaggactctgggttagg

SEQ ID NO: 171 - DNA, 18 bp, Homo sapiens
cctaggactctgggttag

SEQ ID NO: 172 - DNA, 18 bp, Homo sapiens
tcctaggactctgggtta

SEQ ID NO: 173 - DNA, 18 bp, Homo sapiens
gtcctaggactctgggtt

SEQ ID NO: 174 - DNA, 18 bp, Homo sapiens
agtcctaggactctgggt

SEQ ID NO: 175 - DNA, 18 bp, Homo sapiens
gagtcctaggactctggg

SEQ ID NO: 176 - DNA, 18 bp, Homo sapiens
ggagtcctaggactctgg

SEQ ID NO: 177 - DNA, 18 bp, Homo sapiens
ctggagtcctaggactct

SEQ ID NO: 178 - DNA, 18 bp, Homo sapiens
gctggagtcctaggactc

SEQ ID NO: 179 - DNA, 18 bp, Homo sapiens
ggctggagtcctaggact

SEQ ID NO: 180 - DNA, 18 bp, Homo sapiens
aggctggagtcctaggac

SEQ ID NO: 181 - DNA, 18 bp, Homo sapiens
gaggctggagtcctagga

SEQ ID NO: 182 - DNA, 18 bp, Homo sapiens
ggaggctggagtcctagg

SEQ ID NO: 183 - DNA, 18 bp, Homo sapiens
tggaggctggagtcctag

SEQ ID NO: 184 - DNA, 18 bp, Homo sapiens
ttggaggctggagtccta

SEQ ID NO: 185 - DNA, 18 bp, Homo sapiens
gttggaggctggagtcct

SEQ ID NO: 186 - DNA, 18 bp, Homo sapiens
tgttggaggctggagtcc

SEQ ID NO: 187 - DNA, 18 bp, Homo sapiens
gtgttggaggctggagtc

SEQ ID NO: 188 - DNA, 18 bp, Homo sapiens
ggtgttggaggctggagt

SEQ ID NO: 189 - DNA, 18 bp, Homo sapiens
aggtgttggaggctggag

SEQ ID NO: 190 - DNA, 20 bp, Homo sapiens
aacacgcttgccactaaagc

SEQ ID NO: 191 - DNA, 20 bp, Homo sapiens
agtgggagttgaccaccttg

SEQ ID NO: 192 - DNA, 18 bp, Homo sapiens
caacactcaccttggcct

SEQ ID NO: 193 - DNA, 18 bp, Homo sapiens
cacaacactcaccttggc

SEQ ID NO: 194 - DNA, 18 bp, Homo sapiens
ggcacaacactcaccttg

SEQ ID NO: 195 - DNA, 18 bp, Homo sapiens
agggcacaacactcacct

(19) United States
(12) Patent Application Publication
(10) Pub. No.: US 2023/0174984 A1
(43) Pub. Date: Jun. 8, 2023

(54) COMPOSITIONS AND METHODS FOR TREATING DISORDERS ASSOCIATED WITH LOSS-OF-FUNCTION MUTATIONS IN SYNGAP1

(71) Applicant: The Florey Indtitute of Neuroscience and Mental Health, Parkville (AU)

(72) Inventor: Steven Petrou, Eltham (AU)

(21) Appl. No.: 17/924,255

(22) PCT Filed: May 11, 2021
(86) PCT No.: PCT/AU2021/050436

§ 371 Date: Nov. 9, 2022

(30) Foreign Application Priority Data
May 11, 2020 (AU) 2020901507

(51) Int. Cl. C12N 15/113; C07K 14/47

(52) U.S. Cl. CPC C12N 15/113; C07K 14/4706; C12N 2310/11; C12N 2310/315; C12N 2310/321

(57) ABSTRACT

The present disclosure relates generally to compositions and methods suitable for treating a disorder associated with loss-of-function mutations in SYNGAP1. More specifically, the disclosure relates to methods for treating a disorder associated with heterozygous loss-of-function mutations of SYNGAP1, and to antisense oligonucleotides specific for SYNGAP1 and their use for treating a disorder associated with heterozygous loss-of-function mutations of SYNGAP1.

### RELATED APPLICATIONS

This application claims priority to Australian Provisional Application No. 2020901507 entitled “Compositions and methods for treating disorders associated with loss-of-function mutations in syngap1”, filed on 11 May 2020, the entire content of which is hereby incorporated herein by reference in its entirety.

### FIELD OF INVENTION

The present disclosure relates generally to compositions and methods suitable for treating a disorder associated with loss-of-function mutations in SYNGAP1. More specifically, the disclosure relates to antisense oligonucleotides specific for SYNGAP1 and their use for treating a disorder associated with heterozygous loss-of-function mutations of SYNGAP1.

### BACKGROUND TO THE INVENTION

The SynGAP1 protein (also referred to as SynGAP, Synaptic Ras GTPase-activating protein 1, Ras/Rap GTPase-activating protein SynGAP, Neuronal RasGAP, or Synaptic Ras-GAP 1) is encoded by SYNGAP1 on chromosome 6 at 6p21.32 (HGNC:11497; NCBI gene:8831; NCBI Reference Sequence: NG_016137.2). SynGAP1 is a major component protein of the post synaptic dense matrix and is involved in the N-methyl-D-aspartate receptor (NMDAR)-mediated signal transduction (Rumbaugh et al, 2006, Proc. Natl. Acad. Sci. USA. 103, 4344-4351). It is primarily expressed in the brain (mostly forebrain structures such as the cortex, hippocampus and olfactory bulb).

The SynGAP1 primary transcript is alternatively spliced at several sites to generate at least four C-terminal isoforms: SynGAP-α1, SynGAP1-α2, SynGAP1-β, and SynGAP1-γ. SynGAP1-α1 and SynGAP1-α2 isoforms skip exon 19 and are produced by selective splicing of exon 20, such that SynGAP1-α1 contains a PDZ ligand (−QTRV) and SynGAP1-α2 lacks this domain. The SynGAP1-β isoform includes a frameshifting extension of exon 18 leading to early termination, while the SynGAP1-γ isoform includes exon 19, which contains a short coding sequence followed by a STOP codon. These isoforms appear to have varying functions and may play different roles during development (Araki et al. 2020. bioRxiv 2020.01.28.922013). There are also at least three N-terminal isoforms (A-C) which are a consequence of transcriptional start site usage (for review, see e.g. Gamache et al., 2020, J Neurosc. 40(8):1596-1605).

Heterozygous loss-of-function mutations (e.g. nonsense mutations, large deletion and frameshift mutations) in SYNGAP1 can result in the formation of a truncated transcript, leading to the haploinsufficiency. By virtue of its role as a negative regulator of AMPAR insertion in the postsynaptic membrane, heterozygous mutations in SYNGAP1 and the resulting haploinsufficiency leads to neurodevelopmental defects that include altered dendritic spine and neuronal circuit formation. The variety of symptoms resulting from heterozygous loss-of-function mutations in SYNGAP1 can be classified as a single disorder: mental retardation, autosomal dominant 5 (MRD5). Onset of MRD5 is in the first year of life and the clinical features may be found in different combinations. Most, but not all, patients suffer from epileptic seizures (e.g. myoclonic seizures, reflex seizures, and drop attacks). Other clinical characteristics can include hypotonia, unsteady gait, strabismus, hip dysplasia and some dysmorphic features (e.g. myopathic facial appearance, broad nasal bridge, long nose and full lower lip vermilion). While all patients with SYNGAP1 loss-of-function mutations exhibit some form of intellectual disability or developmental delay (generally moderate to severe, although mild in some cases), around half are also diagnosed with autism spectrum disorder. The vast majority of heterozygous loss-of-function mutations in SYNGAP1 are de novo mutations.

The only therapies currently available to patients with MRD5 or any other disorder associated with a heterozygous loss-of-function mutation in SYNGAP1 are those that treat a symptom of the disorder, such as agents to treat epileptic seizures or interventions (e.g. speech therapy, physiotherapy, occupational therapy, etc.) to treat the behavioural or developmental symptoms of ASD, intellectual disability or developmental delay. Consequently, there remains a need for agents, compositions and methods for the treatment of MRD5 or any other disorder associated with a heterozygous loss-of-function mutation in SYNGAP1.

### SUMMARY OF THE DISCLOSURE

The present disclosure is predicated, at least in part, on the determination that a number of introns are retained in mature SynGAP1 mRNA in brain tissue, including introns 5, 8, 9 12, 13 and 14. Introns 8 and 9 in particular have relatively high retention rates.

Intron retention is a form of gene regulation that serves to direct intron-harbouring transcripts to nonsense-mediated decay, thereby reducing gene expression (Kurosaki & Maquat, 2016, J Cell Sci. 129 (3): 461-467). Intron-retaining transcripts have also been shown to serve as a reservoir of RNAs that undergo splicing and translation whenever their expression is required (Jacob & Smith, 2017, Hum Genet. 136 (9): 1043-1057). The process of transcription, which occurs at a rate of 1-4 kb/min, is especially rate-limiting for neuronal activation following neuronal stimuli (Darzarcq et al, 2007, Nat Strut. Mol. Biol. 14, 796-806). In contrast, splicing of retained introns is a much faster process, taking just seconds to a few minutes (Bayer and Osheim, 1988, Genes Dev. 2, 754-765; Singh and Padgett, 2009, Nat. Strut. Mol. Biol. 16, 1128-1133). Consequently, neurons can achieve a faster mode of gene regulation using intron retention and subsequent splicing and translation as compared to de novo transcription and translation. Intron retention has been demonstrated to occur in a pool of polyadenylated transcripts that are retained in the nucleus. Following neuronal stimulation, they undergo intron excision and are transported to the cytoplasm for further processing, thereby aiding in faster gene regulation.

As demonstrated herein, a retained intron in SynGAP1 mRNA or pre-RNA (such as polyadenylated SynGAP1 mRNA or pre-mRNA transcripts in the nucleus of a cell) can be targeted with antisense oligonucleotides so as to enhance splicing at the splice site of the retained intron, resulting in an increase in the amount of fully-spliced SynGAP1 mRNA. Consequently, the antisense oligonucleotides provided herein are useful for increasing the amount of SynGAP1 produced by a cell. The antisense oligonucleotides provided herein are therefore also useful as therapeutic agents for the treatment of diseases or disorders associated with heterozygous loss-of-function mutations in SYNGAP1, such as autosomal mental retardation type 5 (or SYNGAP1-related intellectual disability) wherein increasing the levels of SynGAP1 protein can provide a therapeutic effect. For example, targeting intron 8 and/or intron 9 with antisense oligonucleotides can increase the amount of fully-spliced SynGAP1 mRNA (as demonstrated herein), more so than targeting other introns, and thus may be particularly useful for treating disorders associated with a heterozygous loss-of-function mutation in SYNAGP1.

Accordingly, in one aspect, provided is a method for increasing levels of SynGAP1 protein in a cell, comprising contacting the cell with an antisense oligonucleotide that enhances splicing at a splice site of a retained intron in an intron-retaining SynGAP1 mRNA or pre-mRNA, wherein the retained intron is selected from among intron 5, 8, 9, 12, 13 and 14 and wherein the antisense oligonucleotide comprises a sequence of nucleobases that is complementary to a target region in the SynGAP1 mRNA or pre-mRNA.

In another aspect, provided is a method for increasing levels of SynGAP1 protein in a subject, comprising administering to the subject an antisense oligonucleotide that enhances splicing at a splice site of a retained intron in an intron-retaining SynGAP1 mRNA or pre-mRNA, wherein the retained intron is selected from among intron 5, 8, 9, 12, 13 and 14 and wherein the antisense oligonucleotide comprises a sequence of nucleobases that is complementary to a target region in the SynGAP1 mRNA or pre-mRNA. In some embodiments, the subject has a heterozygous loss-of-function mutation in SYNAGP1.

In some examples, the subject has a disorder associated with a heterozygous loss-of-function mutation in SYNAGP1, such as mental retardation, autosomal dominant 5 (MRD5), autism or intellectual disability.

Also provided is a method for treating a disorder associated with a heterozygous loss-of-function mutation in SYNAGP1, comprising administering to the subject an antisense oligonucleotide that enhances splicing at a splice site of a retained intron in an intron-retaining SynGAP1 mRNA or pre-mRNA, wherein the retained intron is selected from among intron 5, 8, 9, 12, 13 and 14, and wherein the antisense oligonucleotide comprises a sequence of nucleobases that is complementary to a target region in the SynGAP1 mRNA or pre-mRNA. In particular examples, the disorder is mental retardation, autosomal dominant 5 (MRD5), autism or intellectual disability.

In some embodiments of the methods of the disclosure, the antisense oligonucleotide binds to, or adjacent to, an intron splicing silencer (ISS); binds to nucleotides within a G-quadruplex; or binds to nucleotides with an RNA secondary structure. The ISS may be recognised by a heterogeneous nuclear ribonucleoprotein (hnRNP), such as hnRNPA1 or hnRNP I.

In one example, the retained intron is intron 8 and the ISS is at positions +17-22, +23-28, +17-28, or +57-62 relative to the 5′ splice site of intron 8.

In particular embodiments, the retained intron is intron 8 and the target region spans positions +4-35, +5-35, +6-35, +7-35, +8-35, +9-35, +10-35, +11-35, +12-35, +13-35, +4-34, +5-34, +6-34, +7-34, +8-34, +9-34, +10-34, +11-34, +12-34, +13-34, +4-33, +5-33, +6-33, +7-33, +8-33, +9-33, +10-33, +11-33, +12-33, +13-33, +4-32, +5-32, +6-32, +7-32, +8-32, +9-32, +10-32, +11-32, +12-32, +13-32, +4-31, +5-31, +6-31, +7-31, +8-31, +9-31, +10-31, +11-31, +12-31, +13-31, +4-30, +5-30, +6-30, +7-30, +8-30, +9-30, +10-30, +11-30, +12-30, +13-30, +4-29, +5-29, +6-29, +7-29, +8-29, +9-29, +10-29, +11-29, +12-29, +13-29, +4-28, +5-28, +6-28, +7-28, +8-28, +9-28, +10-28, +11-28, +12-28, +13-28, +4-27, +5-27, +6-27, +7-27, +8-27, +9-27, +10-27, +11-27, +12-27, +13-27, +4-26, +5-26, +6-26, +7-26, +8-26, +9-26, +10-26, +11-26, +12-26, +13-26, +4-25, +5-25, +6-25, +7-25, +8-25, +9-25, +10-25, +11-25, +12-25, +13-25, +4-24, +5-24, +6-24, +7-24, +8-24, +9-24, +10-24, +11-24, +12-24, +13-24, +4-23, +5-23, +6-23, +7-23, +8-23, +9-23+10-23, +11-23, +12-23, +13-23, +4-22, +5-22, +6-22, +7-22, +8-22, +9-22, +10-22, +11-22, +12-22, +13-22, +4-21, +5-21, +6-21, +7-21, +8-21, +9-21, +10-21, +11-21, +12-21, +13-21, +4-20, +5-20, +6-20, +7-20, +8-20, +9-20, +10-20, +11-20, +12-20, +13-20, +4-19, +5-19, +6-19, +7-19, +8-19, +9-19, +10-19, +11-19, +12-19, +13-19, +4-18, +5-18, +6-18, +7-18, +8-18, +9-18, +10-18, or +11-18 relative to the 5′ splice site of intron 8. In further embodiments, the retained intron is intron 8 and the target region spans positions +45-70, +46-70, +47-70, +48-70, +49-70, +50-70, +51-70, +52-70, +53-70, +45-69, +46-69, +47-69, +48-69, +49-69, +50-69, +51-69, +52-69, +53-69, +45-68, +46-68, +47-68, +48-68, +49-68, +50-68, +51-68, +52-68, +53-68, +45-67, +46-67, +47-67, +48-67, +49-67, +50-67, +51-67, +52-67, +53-67, +45-66, +46-66, +47-66, +48-66, +49-66, +50-66, +51-66, +52-66, +53-66, +45-65, +46-65, +47-65, +48-65, +49-65, +50-65, +51-65, +52-65, +53-65, +45-64, +46-64, +47-64, +48-64, +49-64, +50-64, +51-64, +52-64, +53-64, +45-63, +46-63, +47-63, +48-63, +49-63, +50-63, +51-63, +52-63, +53-63, +45-62, +46-62, +47-62, +48-62, +49-62, +50-62, +51-62, +52-62, or +53-62 relative to the 5′ splice site of intron 8.

In some examples, the antisense oligonucleotide comprises a sequence having at least or about 70%, 80%, or 90% sequence identity to a sequence set forth in any one of SEQ ID NOs:83-143, or a sequence having at least 8, 9, 10, 11, 12, 13, 14 or 15 contiguous nucleotides from a sequence set forth in any one of SEQ ID NOs:83-143. In one embodiment, the antisense oligonucleotide comprises the sequence set forth in any one of SEQ ID NOs:91-93, or a sequence comprising at least 8, 9, 10, 11, 12, 13, 14 or 15 contiguous nucleotides from a sequence set forth in any one of SEQ ID NOs:91-93.

In other examples, the retained intron is intron 9 and the ISS is at positions +21-29, +104-108 or +190-195 relative to the 5′ splice site of intron 8.

In particular examples, the retained intron is intron 9 and the target region spans +10-40, +11-40, +12-40, +13-40, +14-40, +15-40, +16-40, +17-40, +18-40, +10-39, +11-39, +12-39, +13-39, +14-39, +15-39, +16-39, +17-39, +18-39, +10-38, +11-38, +12-38, +13-38, +14-38, +15-38, +16-38, +17-38, +18-38, +10-37, +11-37, +12-37, +13-37, +14-37, +15-37, +16-37, +17-37, +18-37, +10-36, +11-36, +12-36, +13-36, +14-36, +15-36, +16-36, +17-36, +18-36, +10-35, +11-35, +12-35, +13-35, +14-35, +15-35, +16-35, +17-35, +18-35, +10-34, +11-34, +12-34, +13-34, +14-34, +15-34, +16-34, +17-34, +18-34, +10-33, +11-33, +12-33, +13-33, +14-33, +15-33, +16-33, +17-33, +18-33, +10-32, +11-32, +12-32, +13-32, +14-32, +15-32, +16-32, +17-32, +18-32, +10-31, +11-31, +12-31, +13-31, +14-31, +15-31, +16-31, +17-31, +18-31, +10-30, +11-30, +12-30, +13-30, +14-30, +15-30, +16-30, +17-30, or +18-30 relative to the 5′ splice site of intron 9. In some examples, the antisense oligonucleotide comprises a sequence having at least or about 70%, 80%, or 90% sequence identity to a sequence set forth in any one of SEQ ID NOs:144-167, or a sequence having at least 8, 9, 10, 11, 12, 13, 14 or 15 contiguous nucleotides from a sequence set forth in any one of SEQ ID NOs:144-167.

In further examples, the retained intron is intron 9 and the target region spans positions +87-120, +88-120, +89-120, +90-120, +91-120, +92-120, +93-120, +94-120, +95-120, +96-120, +97-120, +98-120, +87-119, +88-119, +89-119, +90-119, +91-119, +92-119, +93-119, +94-119, +95-119, +96-119, +97-119, +98-119, +87-118, +88-118, +89-118, +90-118, +91-118, +92-118, +93-118, +94-118, +95-118, +96-118, +97-118, +98-118, +87-117, +88-117, +89-117, +90-117, +91-117, +92-117, +93-117, +94-117, +95-117, +96-117, +97-117, +98-117, +87-116, +88-116, +89-116, +90-116, +91-116, +92-116, +93-116, +94-116, +95-116, +96-116, +97-116, +98-116, +87-115, +88-115, +89-115, +90-115, +91-115, +92-115, +93-115, +94-115, +95-115, +96-115, +97-115, +98-115, +87-114, +88-114, +89-114, +90-114, +91-114, +92-114, +93-114, +94-114, +95-114, +96-114, +97-114, +98-114, +87-113, +88-113, +89-113, +90-113, +91-113, +92-113, +93-113, +94-113, +95-113, +96-113, +97-113, +98-113, +87-112, +88-112, +89-112, +90-112, +91-112, +92-112, +93-112, +94-112, +95-112, +96-112, +97-112, +98-112, +87-111, +88-111, +89-111, +90-111, +91-111, +92-111, +93-111, +94-111, +95-111, +96-111, +97-111, +98-111, +87-110, +88-110, +89-110, +90-110, +91-110, +92-110, +93-110, +94-110, +95-110, +96-110, +97-110, or +98-110 relative to the 5′ splice site of intron 9. In some examples, the antisense oligonucleotide comprises a sequence having at least or about 70%, 80%, or 90% sequence identity to a sequence set forth in any one of SEQ ID NOs: 168-189, or a sequence having at least 8, 9, 10, 11, 12, 13, 14 or 15 contiguous nucleotides from a sequence set forth in any one of SEQ ID NOs: 168-189.

In other examples, the retained intron is intron 9 and the target region spans positions +175-205, +176-205, +177-205, +178-205, +179-205, +180-205, +181-205, +182-205, +183-205, +184-205, +185-205, +175-204, +176-204, +177-204, +178-204, +179-204, +180-204, +181-204, +182-204, +183-204, +184-204, +185-204, +175-203, +176-203, +177-203, +178-203, +179-203, +180-203, +181-203, +182-203, +183-203, +184-203, +185-203, +175-202, +176-202, +177-202, +178-202, +179-202, +180-202, +181-202, +182-202, +183-202, +184-202, +185-202, +175-201, +176-201, +177-201, +178-201, +179-201, +180-201, +181-201, +182-201, +183-201, +184-201, +185-201, +175-200, +176-200, +177-200, +178-200, +179-200, +180-200, +181-200, +182-200, +183-200, +184-200, +185-200, +175-199, +176-199, +177-199, +178-199, +179-199, +180-199, +181-199, +182-199, +183-199, +184-199, +185-199, +175-198, +176-198, +177-198, +178-198, +179-198, +180-198, +181-198, +182-198, +183-198, +184-198, +185-198, +175-197, +176-197, +177-197, +178-197, +179-197, +180-197, +181-197, +182-197, +183-197, +184-197, +185-197, +175-196, +176-196, +177-196, +178-196, +179-196, +180-196, +181-196, +182-196, +183-196, +184-196, +185-196, +175-195, +176-195, +177-195, +178-195, +179-195, +180-195, +181-195, +182-195, +183-195, +184-195, or +185-195, relative to the 5′ splice site of intron 9.

In the methods of the present disclosure, the antisense oligonucleotide may consist of, for example, from 8 to 50, 8 to 40, 8 to 35, 8 to 30, 8 to 25, 8 to 20, 8 to 15, 9 to 50, 9 to 40, 9 to 35, 9 to 30, 9 to 25, 9 to 20, 9 to 15, 10 to 50, 10 to 40, 10 to 35, 10 to 30, 10 to 25, 10 to 20, 10 to 15, 11 to 50, 11 to 40, 11 to 35, 11 to 30, 11 to 25, 11 to 20, 11 to 15, 12 to 50, 12 to 40, 12 to 35, 12 to 30, 12 to 25, 12 to 20, or 12 to 15 nucleobases. In some embodiments, the antisense oligonucleotide is at least 70%, 80%, 85%, 86%, 87%, 88%, 89%, 90%, 91%, 92%, 93%, 94%, 95%, 96%, 97%, 98%, or 99% complementary to the target region. In particular embodiments, the antisense oligonucleotide comprises least 8, 9, 10, 11, 12, 13, 14, 15, 16, 17, 18, 19 or 20 contiguous nucleobases that are 100% complementary to the target region.

The antisense oligonucleotide utilised in the methods of the present disclosure may comprise at least one modification, e.g. a nucleobase modification, a modification of the oligonucleotide backbone or a modification of a ribose sugar. In one example, the antisense oligonucleotide comprises a modified sugar selected from among a 2′-O-methyl (20Me), 2′-O-methoxy-ethyl (MOE), locked nucleic acids (LNA), 2′-fluoro or S-constrained-ethyl (cEt). In a further example, the antisense oligonucleotide comprises backbone that comprises phosphorothioates. In additional embodiments, the antisense oligonucleotide activates RNase H.

In the methods of the disclosure that include administering to the subject an antisense oligonucleotide, the subject may first be determined to have a heterozygous loss-of-function mutation in SYNAGP1. In particular examples, the subject has been genotyped to identify a heterozygous loss-of-function mutation in SYNAGP1. The antisense oligonucleotide may be administered to the subject by parenteral administration (e.g. subcutaneous administration, intravenous administration, intramuscular administration, intraarterial administration, intraperitoneal administration, or intracranial administration) or intranasal administration (e.g. intrathecal or intracerebroventricular administration). In some examples, the antisense oligonucleotide or composition is administered to the subject about every 1, 2, 3, 4, 5, 6, 7, 8, 9, 10, 11, 12 or more months.

In a further aspect, provided herein is an antisense oligonucleotide comprising a sequence of nucleobases that is complementary to a target region in an intron-retaining SynGAP1 mRNA or pre-mRNA, wherein the target region is in a retained intron and wherein the retained intron is selected from among intron 5, 8, 9, 12, 13 or 14.

In some embodiments, the antisense oligonucleotide binds to, or adjacent to, an intron splicing silencer (ISS); binds to nucleotides within a G-quadruplex; or binds to nucleotides with an RNA secondary structure. In a particular example, the ISS is recognised by a heterogeneous nuclear ribonucleoproteins (hnRNP), e.g. hnRNPA1 or hnRNP I.

In one example, the retained intron in which the target region is present is intron 8 and the ISS is at positions +17-22, +23-28, +17-28, or +57-62 relative to the 5′ splice site of intron 8.

In particular embodiments, the retained intron is intron 8 and the target region spans positions +4-35, +5-35, +6-35, +7-35, +8-35, +9-35, +10-35, +11-35, +12-35, +13-35, +4-34, +5-34, +6-34, +7-34, +8-34, +9-34, +10-34, +11-34, +12-34, +13-34, +4-33, +5-33, +6-33, +7-33, +8-33, +9-33, +10-33, +11-33, +12-33, +13-33, +4-32, +5-32, +6-32, +7-32, +8-32, +9-32, +10-32, +11-32, +12-32, +13-32, +4-31, +5-31, +6-31, +7-31, +8-31, +9-31, +10-31, +11-31, +12-31, +13-31, +4-30, +5-30, +6-30, +7-30, +8-30, +9-30, +10-30, +11-30, +12-30, +13-30, +4-29, +5-29, +6-29, +7-29, +8-29, +9-29, +10-29, +11-29, +12-29, +13-29, +4-28, +5-28, +6-28, +7-28, +8-28, +9-28, +10-28, +11-28, +12-28, +13-28, +4-27, +5-27, +6-27, +7-27, +8-27, +9-27, +10-27, +11-27, +12-27, +13-27, +4-26, +5-26, +6-26, +7-26, +8-26, +9-26, +10-26, +11-26, +12-26, +13-26, +4-25, +5-25, +6-25, +7-25, +8-25, +9-25, +10-25, +11-25, +12-25, +13-25, +4-24, +5-24, +6-24, +7-24, +8-24, +9-24, +10-24, +11-24, +12-24, +13-24, +4-23, +5-23, +6-23, +7-23, +8-23, +9-23+10-23, +11-23, +12-23, +13-23, +4-22, +5-22, +6-22, +7-22, +8-22, +9-22, +10-22, +11-22, +12-22, +13-22, +4-21, +5-21, +6-21, +7-21, +8-21, +9-21, +10-21, +11-21, +12-21, +13-21, +4-20, +5-20, +6-20, +7-20, +8-20, +9-20, +10-20, +11-20, +12-20, +13-20, +4-19, +5-19, +6-19, +7-19, +8-19, +9-19, +10-19, +11-19, +12-19, +13-19, +4-18, +5-18, +6-18, +7-18, +8-18, +9-18, +10-18, or +11-18 relative to the 5′ splice site of intron 8. In further embodiments, the retained intron is intron 8 and the target region spans positions +45-70, +46-70, +47-70, +48-70, +49-70, +50-70, +51-70, +52-70, +53-70, +45-69, +46-69, +47-69, +48-69, +49-69, +50-69, +51-69, +52-69, +53-69, +45-68, +46-68, +47-68, +48-68, +49-68, +50-68, +51-68, +52-68, +53-68, +45-67, +46-67, +47-67, +48-67, +49-67, +50-67, +51-67, +52-67, +53-67, +45-66, +46-66, +47-66, +48-66, +49-66, +50-66, +51-66, +52-66, +53-66, +45-65, +46-65, +47-65, +48-65, +49-65, +50-65, +51-65, +52-65, +53-65, +45-64, +46-64, +47-64, +48-64, +49-64, +50-64, +51-64, +52-64, +53-64, +45-63, +46-63, +47-63, +48-63, +49-63, +50-63, +51-63, +52-63, +53-63, +45-62, +46-62, +47-62, +48-62, +49-62, +50-62, +51-62, +52-62, or +53-62 relative to the 5′ splice site of intron 8.

In some examples, the antisense oligonucleotide comprises a sequence having at least or about 70%, 80%, or 90% sequence identity to a sequence set forth in any one of SEQ ID NOs:83-143, or a sequence having at least 8, 9, 10, 11, 12, 13, 14 or 15 contiguous nucleotides from a sequence set forth in any one of SEQ ID NOs:83-143. In one embodiment, the antisense oligonucleotide comprises the sequence set forth in any one of SEQ ID NOs:91-93, or a sequence comprising at least 8, 9, 10, 11, 12, 13, 14 or 15 contiguous nucleotides from a sequence set forth in any one of SEQ ID NOs:91-93.

In further embodiments, the retained intron is intron 8 and the target region spans positions +45-70, +46-70, +47-70, +48-70, +49-70, +50-70, +51-70, +52-70, +53-70, +45-69, +46-69, +47-69, +48-69, +49-69, +50-69, +51-69, +52-69, +53-69, +45-68, +46-68, +47-68, +48-68, +49-68, +50-68, +51-68, +52-68, +53-68, +45-67, +46-67, +47-67, +48-67, +49-67, +50-67, +51-67, +52-67, +53-67, +45-66, +46-66, +47-66, +48-66, +49-66, +50-66, +51-66, +52-66, +53-66, +45-65, +46-65, +47-65, +48-65, +49-65, +50-65, +51-65, +52-65, +53-65, +45-64, +46-64, +47-64, +48-64, +49-64, +50-64, +51-64, +52-64, +53-64, +45-63, +46-63, +47-63, +48-63, +49-63, +50-63, +51-63, +52-63, +53-63, +45-62, +46-62, +47-62, +48-62, +49-62, +50-62, +51-62, +52-62, or +53-62 relative to the 5′ splice site of intron 8.

In other examples, the retained intron is intron 9 and the ISS is at positions +21-29, +104-108 or +190-195 relative to the 5′ splice site of intron 8.

In particular examples, the retained intron is intron 9 and the target region spans +10-40, +11-40, +12-40, +13-40, +14-40, +15-40, +16-40, +17-40, +18-40, +10-39, +11-39, +12-39, +13-39, +14-39, +15-39, +16-39, +17-39, +18-39, +10-38, +11-38, +12-38, +13-38, +14-38, +15-38, +16-38, +17-38, +18-38, +10-37, +11-37, +12-37, +13-37, +14-37, +15-37, +16-37, +17-37, +18-37, +10-36, +11-36, +12-36, +13-36, +14-36, +15-36, +16-36, +17-36, +18-36, +10-35, +11-35, +12-35, +13-35, +14-35, +15-35, +16-35, +17-35, +18-35, +10-34, +11-34, +12-34, +13-34, +14-34, +15-34, +16-34, +17-34, +18-34, +10-33, +11-33, +12-33, +13-33, +14-33, +15-33, +16-33, +17-33, +18-33, +10-32, +11-32, +12-32, +13-32, +14-32, +15-32, +16-32, +17-32, +18-32, +10-31, +11-31, +12-31, +13-31, +14-31, +15-31, +16-31, +17-31, +18-31, +10-30, +11-30, +12-30, +13-30, +14-30, +15-30, +16-30, +17-30, or +18-30 relative to the 5′ splice site of intron 9. In some examples, the antisense oligonucleotide comprises a sequence having at least or about 70%, 80%, or 90% sequence identity to a sequence set forth in any one of SEQ ID NOs:144-167, or a sequence having at least 8, 9, 10, 11, 12, 13, 14 or 15 contiguous nucleotides from a sequence set forth in any one of SEQ ID NOs:144-167.

In further examples, the retained intron is intron 9 and the target region spans positions +90-120, +91-120, +92-120, +93-120, +94-120, +95-120, +96-120, +97-120, +98-120, +90-119, +91-119, +92-119, +93-119, +94-119, +95-119, +96-119, +97-119, +98-119, +90-118, +91-118, +92-118, +93-118, +94-118, +95-118, +96-118, +97-118, +98-118, +90-117, +91-117, +92-117, +93-117, +94-117, +95-117, +96-117, +97-117, +98-117, +90-116, +91-116, +92-116, +93-116, +94-116, +95-116, +96-116, +97-116, +98-116, +90-115, +91-115, +92-115, +93-115, +94-115, +95-115, +96-115, +97-115, +98-115, +90-114, +91-114, +92-114, +93-114, +94-114, +95-114, +96-114, +97-114, +98-114, +90-113, +91-113, +92-113, +93-113, +94-113, +95-113, +96-113, +97-113, +98-113, +90-112, +91-112, +92-112, +93-112, +94-112, +95-112, +96-112, +97-112, +98-112, +90-111, +91-111, +92-111, +93-111, +94-111, +95-111, +96-111, +97-111, +98-111, +90-110, +91-110, +92-110, +93-110, +94-110, +95-110, +96-110, +97-110, or +98-110 relative to the 5′ splice site of intron 9. In some examples, the antisense oligonucleotide comprises a sequence having at least or about 70%, 80%, or 90% sequence identity to a sequence set forth in any one of SEQ ID NOs: 168-189, or a sequence having at least 8, 9, 10, 11, 12, 13, 14 or 15 contiguous nucleotides from a sequence set forth in any one of SEQ ID NOs: 168-189.

In other examples, the retained intron is intron 9 and the target region spans positions +175-205, +176-205, +177-205, +178-205, +179-205, +180-205, +181-205, +182-205, +183-205, +184-205, +185-205, +175-204, +176-204, +177-204, +178-204, +179-204, +180-204, +181-204, +182-204, +183-204, +184-204, +185-204, +175-203, +176-203, +177-203, +178-203, +179-203, +180-203, +181-203, +182-203, +183-203, +184-203, +185-203, +175-202, +176-202, +177-202, +178-202, +179-202, +180-202, +181-202, +182-202, +183-202, +184-202, +185-202, +175-201, +176-201, +177-201, +178-201, +179-201, +180-201, +181-201, +182-201, +183-201, +184-201, +185-201, +175-200, +176-200, +177-200, +178-200, +179-200, +180-200, +181-200, +182-200, +183-200, +184-200, +185-200, +175-199, +176-199, +177-199, +178-199, +179-199, +180-199, +181-199, +182-199, +183-199, +184-199, +185-199, +175-198, +176-198, +177-198, +178-198, +179-198, +180-198, +181-198, +182-198, +183-198, +184-198, +185-198, +175-197, +176-197, +177-197, +178-197, +179-197, +180-197, +181-197, +182-197, +183-197, +184-197, +185-197, +175-196, +176-196, +177-196, +178-196, +179-196, +180-196, +181-196, +182-196, +183-196, +184-196, +185-196, +175-195, +176-195, +177-195, +178-195, +179-195, +180-195, +181-195, +182-195, +183-195, +184-195, or +185-195, relative to the 5′ splice site of intron 9.

In some embodiments, the antisense oligonucleotide may consist of, for example, from 8 to 50, 8 to 40, 8 to 35, 8 to 30, 8 to 25, 8 to 20, 8 to 15, 9 to 50, 9 to 40, 9 to 35, 9 to 30, 9 to 25, 9 to 20, 9 to 15, 10 to 50, 10 to 40, 10 to 35, 10 to 30, 10 to 25, 10 to 20, 10 to 15, 11 to 50, 11 to 40, 11 to 35, 11 to 30, 11 to 25, 11 to 20, 11 to 15, 12 to 50, 12 to 40, 12 to 35, 12 to 30, 12 to 25, 12 to 20, or 12 to 15 nucleobases. In some embodiments, the antisense oligonucleotide is at least 70%, 80%, 85%, 86%, 87%, 88%, 89%, 90%, 91%, 92%, 93%, 94%, 95%, 96%, 97%, 98%, or 99% complementary to the target region. In particular embodiments, the antisense oligonucleotide comprises least 8, 9, 10, 11, 12, 13, 14, 15, 16, 17, 18, 19 or 20 contiguous nucleobases that are 100% complementary to the target region.

The antisense oligonucleotide may comprise at least one modification, e.g. a nucleobase modification, a modification of the oligonucleotide backbone or a modification of a ribose sugar. In one example, the antisense oligonucleotide comprises a modified sugar selected from among a 2′-O-methyl (20Me), 2′-O-methoxy-ethyl (MOE), locked nucleic acids (LNA), 2′-fluoro or S-constrained-ethyl (cEt). In a further example, the antisense oligonucleotide comprises backbone that comprises phosphorothioates. In additional embodiments, the antisense oligonucleotide activates RNase H.

Also provided are compositions comprising an antisense oligonucleotide of the present disclosure, such as pharmaceutical compositions.

In a further aspect, provided is a use of an antisense oligonucleotide for the treatment of a disorder associated with a heterozygous loss-of-function mutation in SYNAGP1, wherein the antisense oligonucleotide enhances splicing at a splice site of a retained intron in an intron-retaining SynGAP1 mRNA or pre-mRNA, wherein the retained intron is selected from among intron 5, 8, 9, 12, 13 and 14 and wherein the antisense oligonucleotide comprises a sequence of nucleobases that is complementary to a target region in the SynGAP1 mRNA or pre-mRNA.

### BRIEF DESCRIPTION OF THE DRAWINGS

FIG. 1 is a schematic representation of intron retention in SYNGAP1, as analysed from information obtained from IRBase. The top panel shows a genomic map of the Syngap1 gene from UCSC browser. Thin lines represent the introns and the thick lines/blocks correspond to the exons. The bottom panel shows intron-retention events corresponding to introns in the genomic map. The height of the bars is indicative of the number of recorded events.

FIG. 2 is a schematic showing the design of primers for each exon-intron pair across the SynGAP1 sequence. A. Primers that are specific for intron-retaining transcripts: The forward primer was designed from the sequence of the preceding exon and the reverse primer from the sequence of the intron downstream to the exon. B. Primers specific to spliced transcripts: One of the primers was designed such that it spanned the junction of two nearby exons, while the other was designed from the sequence of the preceding or the succeeding exon accordingly.

FIG. 3 shows relative expression of introns in whole brain SynGAP1 mRNA obtained from two commercial sources. The expression of individual introns across the entire transcript was compared with the averaged exon expression. The results are a representation of three experiments, with the standard error of the mean indicated. A. mRNA from source 1—Ambion. B. mRNA from source 2—Takara.

FIG. 4 shows relative expression of introns in SynGAP1 mRNA from cell lines. The expression of individual introns across the entire transcript was compared with the averaged exon expression. The results are a representation of three experiments, with the standard error of the mean indicated. A. mRNA from SH-SY5Y cells. B. mRNA from SK-N-AS cells. C. mRNA from ARPE19 cells.

FIG. 5 is a schematic of the secondary structure prediction of a sequence of an intron of Syngap1.

FIG. 6 is a graphical representation of modulation of SynGAP1 transcript expression in the presence of antisense oligonucleotides. Effect of antisense oligonucleotides targeting the 5 prime end of SynGAP1 intron 8 on its expression. The antisense oligonucleotides were transfected into ARPE19 cells at a concentration of 200 nM. Following 24 hours incubation, the expression of Syngap1 was analysed by qPCR. Mock transfected cells were used as a negative control. The housekeeping gene GUSB was used for normalization. The number of biological replicates ranges from 3 to 9. The sequence targeted by the antisense oligonucleotides that induce upregulation is given below the x-axis.

FIG. 7 is a photographic representation of PCR products following amplification of RNA prepared from antisense oligonucleotide-treated cells. The effect of antisense oligonucleotides on the expression of the intron-retaining transcripts (Syngap1 E8-I8-E9) and on the mature transcripts (Syngap1 E8-E9) is shown.

FIG. 8 is a graphical representation of the effect of varying of the antisense oligonucleotides (ASOs; SYN-INT8+10, SYN-INT8+11) on Syngap1 expression. Following 24 hours incubation of ASOs, the expression of Syngap1 was analysed by qPCR. Mock transfected cells were used as a negative control. The housekeeping gene GUSB was used for normalization. The bars in the graph represent, from left to right for each ASO, 80 nM, 200 nM, 500 nM and 1000 nM ASO.

FIG. 9 is a graphical representation of the effect of varying treatment time periods of the ASOs (SYN-INT8+10, SYN-INT8+11) on Syngap1 expression. Following 24-96 hours ASO incubation, the expression of Syngap1 was analysed by qPCR. Mock transfected cells were used as a negative control. The housekeeping gene GUSB was used for normalization. The bars in the graph represent, from left to right for each ASO, 24 hr, 48 hr, 72 hr and 96 hr treatment using each ASO.

FIG. 10 is a graphical representation of modulation of SynGAP1 transcript expression in the presence of antisense oligonucleotides. Effect of antisense oligonucleotides targeting SynGAP1 intron 9 on its expression. The antisense oligonucleotides were transfected into ARPE19 cells at a concentration of 200 nM. Following 24 hours incubation, the expression of Syngap1 was analysed by qPCR. Mock transfected cells were used as a negative control. The housekeeping gene GUSB was used for normalization. The number of biological replicates ranges from 3 to 9. The sequence targeted by the antisense oligonucleotides that induce upregulation is given below the x-axis.

FIG. 11 is a photographic and graphical representation of PCR products following amplification of RNA prepared from cells treated with the ASO SYN-INT8+11 or SYN-INT9+89. A. Semi-quantitative PCR of cDNA prepared from ASO-treated ARPE19 cells. The primers used bound within the flanking exons (E8-E9) of intron 8 and (E9-E10) intron 9. The PCR products were separated on an agarose gel. The samples were ran as biological duplicates. B. Graphical representation of the quantification by Image J of 3 semi-quantitative PCR experiments assessing intron 8 transcripts following treatment with ASO SYN-INT8+11. C. Graphical representation of the quantification by Image J of 3 semi-quantitative PCR experiments assessing intron 9 transcripts following treatment with ASO SYN-INT9+89.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e. at least one or more than one) unless the context clearly dictates otherwise. Thus, for example, reference to “a polypeptide” includes a single polypeptide, as well as two or more polypeptides.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

An “antisense oligonucleotide” refers to a single-stranded oligonucleotide having a sequence that permits hybridization to a corresponding region or segment of a target nucleic acid. Reference to an antisense oligonucleotide includes reference to both unmodified and modified antisense oligonucleotides, wherein a modified antisense oligonucleotide contains at least one modified nucleoside and/or modified internucleoside linkage.

“Complementary,” as used herein, refers to the capacity for precise pairing between two nucleobases, such as between a nucleobase in an antisense oligonucleotide and a nucleobase in a SynGAP1 mRNA or pre-mRNA. The antisense oligonucleotide and the mRNA or pre-mRNA are complementary to each other when a sufficient number of corresponding positions in each molecule are occupied by nucleobases which can hydrogen bond with each other. Thus, “complementary” is used to indicate a sufficient degree of precise pairing over a sufficient number of nucleotides such that stable and specific binding occurs between the antisense oligonucleotide and the mRNA or pre-mRNA. It is understood that the antisense oligonucleotide need not be 100% complementary to the target region in the SYNGAP1 mRNA or pre-mRNA to hybridize thereto. Moreover, an oligonucleotide may be complementary to, and hybridize, over one or more segments such that intervening or adjacent segments are not involved in the hybridization event. “Complementary” as used herein therefore includes reference to less than 100% complementary, such at least or about 70%, 75%, 80%, 85%, 90% or 95% sequence complementarity.

As used herein, a “disorder associated with a loss-of-function mutation in SYNGAP1” refers to a disorder that is associated with, is partially or completely caused by, or has one or more symptoms that are partially or completely caused by, a mutation in SYNGAP1 that results in a loss-of-function phenotype, i.e. an decrease in the level (or amount) or activity of SynGAP1.

As used herein, “expression of SynGAP1” refers to the transcription of mRNA from SYNGAP1 or the translation of protein from the SynGAP1 mRNA. SynGAP1 expression can be assessed using any method known in the art, including, but not limited to, Northern blot, Western blot and qRT-PCR.

As used herein, a “loss-of-function mutation” is a mutation in SYNGAP1 that results in a decrease in expression and/or activity of the encoded SynGAP1 protein. Expression of the encoded SynGAP1 protein can be assessed using standard assays, such as Western blot. Typically, a loss-of-function mutation results in a decrease of at least or about 20%, 30%, 40%, 50%, 60%, 70%, 80%, 90%, 95% or more of the expression and/or activity of the encoded SynGAP1 protein. In some examples, the loss-of-function mutation results in a complete (i.e. 100%) loss of expression or activity of the encoded SynGAP1 protein, such as when the mutation is a mutation (e.g. nonsense mutation, large deletion or frameshift mutation) that results in the formation of a truncated transcript that is either not translated or is translated to a non-functional protein. A “heterozygous loss-of-function mutation” in SYNGAP1 is one that is present in only one copy of SYNGAP1 in the cell (i.e. one allele is a wild-type allele) and can lead to haploinsufficiency.

A “gapmer” as referred to herein is a chimeric antisense oligonucleotide in which an internal region having a plurality of nucleotides that support RNase H cleavage is positioned between external regions having one or more nucleotides, wherein the nucleotides comprising the internal region are chemically distinct from the nucleoside or nucleotides comprising the external regions.

As used herein, “hybridization” or “binding” or grammatical variations thereof means the pairing of substantially complementary strands of nucleic acids, such as between an antisense oligonucleotide of the disclosure and a SynGAP1 mRNA or pre-mRNA. One mechanism of pairing involves hydrogen bonding, which may be Watson-Crick, Hoogsteen or reversed Hoogsteen hydrogen bonding, between complementary nucleobases of the strands of nucleic acids. For example, adenine and thymine or uracil are complementary nucleotides which pair through the formation of hydrogen bonds. Hybridization can occur under varying circumstances. Reference to “hybridizes” or “binds” as used herein means that the antisense oligonucleotide hybridizes or binds to a target region in SynGAP1 mRNA or pre-mRNA by virtue of the complementarity in sequence between the antisense oligonucleotide and the target region, and does not significantly bind to a non-target region.

The terms “linked” and “attached” are used interchangeably and relate to any type of interaction that join two entities, such as an antisense oligonucleotide and a moiety (e.g. a cell penetrating peptide), and include covalent bonds or non-covalent bonds, such as, for example, hydrophobic/hydrophilic interactions, van der Waals forces, ionic bonds or hydrogen bonds.

The term “exon” refers to a portion of a gene that is present in the mature form of mRNA. Exons include the ORF (open reading frame), i.e., the sequence which encodes protein, as well as the 5′ and 3′ UTRs (untranslated regions). The UTRs are important for translation of the protein. Algorithms and computer programs are available for predicting exons in DNA sequences (e.g. Grail, Grail 2 and Genscan and US 20040219522 for determining an exon-intron junctions).

The term “intron” refers to a portion of a gene that is not translated into a wild-type protein and while present in genomic DNA and pre-mRNA, it is generally removed in the formation of mature mRNA by splicing.

The term “messenger RNA” or “mRNA” refers to RNA that is transcribed from genomic DNA and that carries the coding sequence for protein synthesis. The terms “precursor mRNA” or “pre-mRNA” refer to an immature single strand of messenger ribonucleic acid (mRNA) that contains one or more introns and that is directly transcribed from the DNA; for the purposes of the present disclosure, it is considered pre-mRNA until the poly(A) is added and 5′ and 3′ modifications take place. Pre-mRNA is transcribed by an RNA polymerase from a DNA template in the cell nucleus and is comprised of alternating sequences of introns and exons. In eukaryotes, pre-mRNA is processed into mRNA, which includes removal of the introns, i.e., “splicing”, and modifications to the 5′ and 3′ end (e.g., polyadenylation). mRNA typically comprises from 5′ to 3′; a 5′cap (modified guanine nucleotide), 5′ UTR (untranslated region), the coding sequence (beginning with a start codon and ending with a stop codon), the 3′ UTR, and the poly(A) tail. Eukaryotic pre-mRNAs exist only transiently before being processed into mRNA. As described herein, polyadenylated transcripts in the nucleus of a cell can have one or more retained introns even after initial splicing of the primary transcript and addition of the poly(A) tail. For the purposes of the present disclosure, these transcripts are considered mRNA with retained introns. When a pre-mRNA has been properly processed to an mRNA, it is exported out of the nucleus and translated into a protein by ribosomes in the cytoplasm. The term “fully-spliced mRNA” as used herein means that the mRNA does not contain any introns, or does not contain the intron being targeted by the antisense oligonucleotides and methods according to the present disclosure. For the purposes of the present disclosure, where a sequence is provided and is stated as being mRNA, pre-mRNA or RNA (or a region or site within the mRNA, pre-mRNA or RNA), any thymine (T) in the sequence is understood as being a uracil (U).

As used herein, “nucleobase” means a heterocyclic moiety capable of pairing with a base of another nucleic acid, and includes, for example, adenine (A), guanine (G), cytosine (C), thymine (T) and uracil (U). Reference herein to nucleobase also includes reference to a modified nucleobase.

A “nucleoside” as used herein refers to a nucleobase linked to a sugar. Reference herein to a nucleoside also includes reference to a modified nucleoside, which has a modified sugar moiety or modified nucleobase. A “nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics e.g. non furanose sugar units.

As used herein, “nucleotide” refers to a nucleoside having a phosphate group covalently linked to the sugar portion of the nucleoside. Reference herein to a nucleotide also includes reference to a modified nucleotide, which has a modified sugar moiety, modified internucleoside linkage, or modified nucleobase. A “nucleotide mimetic” includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage).

The term “splicing” refers to the modification of a pre-mRNA following transcription, in which introns are removed and exons are joined. Pre-mRNA splicing involves two sequential biochemical reactions. Both reactions involve the spliceosomal transesterification between RNA nucleotides. In a first reaction, the 2′-OH of a specific branch-point nucleotide within an intron, which is defined during spliceosome assembly, performs a nucleophilic attack on the first nucleotide of the intron at the 5′ splice site forming a lariat intermediate. In a second reaction, the 3′-OH of the released 5′ exon performs a nucleophilic attack at the last nucleotide of the intron at the 3′ splice site thus joining the exons and releasing the intron lariat.

As used herein, the term “sequence identity” or “% identical” or grammatical variations means that in a comparison of two sequences over a specified region the two sequences have the specified number or percentage of identical residues in the same position. Sequences can be aligned by any method known to those of skill in the art. Such methods typically maximize matches, and include methods such as using manual alignments and by using the numerous alignment programs available.

The term “splice site” refers to the junction between an exon and an intron in a pre-mRNA molecule (also known as a “splice junction”). The “splice site sequence” is the sequence surround the splice site that is capable of being recognised by the splicing machinery of the cell. A 5′ splice site (also referred to as a splice donor site) is the splice site at the 5′ end of the intron that marks the start of the intron and its boundary with the preceding exon sequence. A 3′ splice site (also referred to as a splice acceptor site) is the splice site at the 3′ end of the intron that marks the end of the intron and its boundary with the following exon sequence. Numbering used herein in reference to a 5′ splice site of an intron is therefore also in reference to the first nucleotide of the intron. Thus, for example, reference to position +1 relative to the 5′ splice site of an intron is reference to the first nucleotide in the intron sequence, e.g. reference to position +1 relative to the 5′ splice site of intron 8 is reference to nucleotide position 1 of the intron 8 sequence, e.g. position 1 of SEQ ID NO:6. In another example, reference to positions +18-27 relative to the 5′ splice site of an intron is reference to the 18th through to the 27th nucleotide position of the intron sequence, e.g. position 18 through to position 27 of the intron 8 set forth in SEQ ID NO:6.

As used herein the terms “treating” or “treatment” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and the like are to be considered in their broadest context. For example, treatment does not necessarily imply that a patient is treated until total recovery. In conditions which display or a characterized by multiple symptoms, the treatment or prevention need not necessarily remedy, prevent, hinder, retard, or reverse all of said symptoms, but may prevent, hinder, retard, or reverse one or more of said symptoms. In the context of the present invention, symptoms that may be ameliorated, reversed, prevented, retarded or the linked include but are not limited to seizures and spasms.

The term “subject” as used herein refers to an animal, in particular a mammal and more particularly a primate including a lower primate and even more particularly, a human who can benefit from the protocol of the present invention. A subject regardless of whether a human or non-human animal or embryo may be referred to as an individual, subject, animal, patient, host or recipient.

### Antisense Oligonucleotides for SYNGAP1

As demonstrated herein, introns 5, 8, 9, 12, 13, 14 and 18 are retained in mature SynGAP1 mRNA in brain tissue. Introns 8 and 9 in particular have relatively high retention rates. As demonstrated herein, a retained intron in SynGAP1 mRNA or pre-RNA can be targeted with antisense oligonucleotides so as to enhance splicing at the splice site of the retained intron, resulting in an increase in the amount of fully-spliced SynGAP1 mRNA (i.e. SynGAP1 mRNA that does not contain any intron). Such antisense oligonucleotides are therefore useful for increasing the amount of SynGAP1 produced by a cell, and thus useful as therapeutic agents for the treatment of disorders associated with heterozygous loss-of-function mutations in SYNGAP1, such as autosomal mental retardation type 5 (or SYNGAP1-related intellectual disability), where increasing the levels of SynGAP1 protein can provide a therapeutic effect.

Thus, provided herein are antisense oligonucleotides that enhance splicing at a splice site of a retained intron in an intron-retaining SynGAP1 mRNA or pre-mRNA, such as intron 5, 8, 9, 12, 13 or 14. In particular examples, the antisense oligonucleotides enhance splicing at a splice site of intron 8 or intron 9 in an intron-retaining SynGAP1 mRNA or pre-mRNA.

The antisense oligonucleotide can function to enhance splicing in one of many ways. In one example, the antisense oligonucleotide binds to, or adjacent to, an intronic splicing silencer (ISS) (also referred to as an ISS site or ISS motif). ISS are cis-acting elements (i.e. sequences) in the RNA that play a role in silencing or inhibiting splicing at a splice site. The ISS is bound by a RNA-binding protein (RBP) that acts as a silencing repressor. Exemplary RBPs that act as repressors include heterogeneous nuclear ribonucleoproteins (hnRNPs), such as hnRNP A1, A2/B1, C1/C2, E1/E2/E3/E4, F, G, H, I, K, L, M, P, Q1/Q2/Q3 and U (for review, see e.g. and hnRNP A2. The motifs recognised and bound by hnRNPs are not necessarily strict consensus sequences in the classical sense, but can be repeat elements (such as in the case of hnRNP L1, which recognises a CA repeat-rich element) or short and degenerate sequences. Moreover, hnRNPs may recognize specific structures rather than linear sequence motifs, such as in the case of hnRNP F (for review, see e.g. Geunes et al., 2016, Hum Genet. 135:851-867; Dvinge, 2018, FEBS Letters, 592:2987-3006). Notwithstanding this, algorithms are available to predict hnRNP binding motifs in RNA molecules (see e.g. Piva et al., 2009, Bioinformatics; Piva et al., 2012, Hum Mutat. 2012 January; 33(1):81-85). Binding of an antisense oligonucleotide to, or adjacent to, an ISS can prevent or inhibit binding of the RBP suppressor (e.g. an hnRNP, such as hnRNP A1), thereby enhancing splicing at a splice site of a retained intron, such as intron 5, 8, 9, 12, 13 or 14. In other examples, the antisense oligonucleotide binds to a site in SynGAP1 mRNA or pre-mRNA that has a propensity to form an RNA secondary structure (e.g. stem, hairpin loop, pseuoknot, bulge, internal loop or multiloop), thereby reducing formation of the structure and facilitating efficient recruitment of splicing factors so as to enhance splicing of the retained-intron. In further examples, the antisense oligonucleotide binds to a sequence involved in the formation of G-quadruplexes, which can stabilise the G-quadruplexe and enhance splicing (see e.g. Rouleau et al., 2015, Nucleic Acids Res. 43(1): 595-606; Ribeiro et al. 2015, Hum Genet. 134(1):37-44).

In some examples, the antisense oligonucleotide of the present disclosure binds to nucleotides (or a target region) within the targeted intron, i.e. the intron for which enhanced splicing is to be effected, e.g. intron 5, 8, 9, 12, 13 or 14. In other examples, the antisense oligonucleotide of the present disclosure binds to nucleotides (or a target region) in an adjacent exon, while still enhancing splicing at a splice site of the targeted intron. As noted above, for the purposes of the present disclosure, for any depiction of the sequence of an mRNA or pre-mRNA, or sequence of a region or site within the mRNA or pre-mRNA, any reference to T is understood to be a reference to U.

In one example, the antisense oligonucleotide binds to a target region within intron 8 in the intron-retaining SynGAP1 mRNA or pre-mRNA. Accordingly, in some examples, an antisense oligonucleotide of the present disclosure has a sequence of nucleobases that is complementary to a sequence of nucleotides within intron 8 of SynGAP1 pre-mRNA, such as within the intron 8 set forth as follows:

(SEQ ID NO: 6)
GTGAGTGTTGTGCCCTCAGGGAAAGGTGACTTGGGAATGGGCACTTGC
TTGGGGGTTAGTGAGGACAGGGCAAATTCACGAGATTGGGTTGTGCAG
AGGCTGACACTTGGATTTTCCTGGGCCTCAG

In some examples, the antisense oligonucleotide binds to (i.e. comprises a sequence that is complementary to) a target region in intron 8 in an intron-retaining SynGAP1 mRNA or pre-mRNA, wherein the target region spans positions +4-100, +4-80, +4-65, +4-30, or +50-70 relative to the 5′ splice site of intron 8. In particular embodiments, the antisense oligonucleotide binds to, or adjacent to, an ISS in intron 8. As determined herein, putative ISS recognised by hnRNPA1 are at positions +17-22 and +23-28 (i.e. spanning +17-28), and +57-62 relative to the 5′ splice site of intron 8 (bolded in the representation of SEQ ID NO:6, above). Thus, in some embodiments, the antisense oligonucleotide binds to, or adjacent to, nucleotides at positions +17-22, +23-28, +17-28, and/or +57-62 relative to the 5′ splice site of intron 8. For example, the antisense oligonucleotide may bind to one or more of the nucleotides at position +4, +5, +6, +7, +8, +9, +10, +11, +12, +13, +14, +15, +16, +17, +18, +19, +20, +21, +22, +23, +24, +25, +26, +27, +28, +29, +30, +31, +32, +33 or +34 relative to the 5′ splice site of intron 8, or may bind to one or more of the nucleotides at position +50, +51, +52, +53, +54, +55, +56, +57, +58, +59, +60, +61, +62, +63, +64 or +65 relative to the 5′ splice site of intron 8.

In one example, the antisense oligonucleotide binds to a target region that spans or is within positions +4-35, +5-35, +6-35, +7-35, +8-35, +9-35, +10-35, +11-35, +12-35, +13-35, +4-34, +5-34, +6-34, +7-34, +8-34, +9-34, +10-34, +11-34, +12-34, +13-34, +4-33, +5-33, +6-33, +7-33, +8-33, +9-33, +10-33, +11-33, +12-33, +13-33, +4-32, +5-32, +6-32, +7-32, +8-32, +9-32, +10-32, +11-32, +12-32, +13-32, +4-31, +5-31, +6-31, +7-31, +8-31, +9-31, +10-31, +11-31, +12-31, +13-31, +4-30, +5-30, +6-30, +7-30, +8-30, +9-30, +10-30, +11-30, +12-30, +13-30, +4-29, +5-29, +6-29, +7-29, +8-29, +9-29, +10-29, +11-29, +12-29, +13-29, +4-28, +5-28, +6-28, +7-28, +8-28, +9-28, +10-28, +11-28, +12-28, +13-28, +4-27, +5-27, +6-27, +7-27, +8-27, +9-27, +10-27, +11-27, +12-27, +13-27, +4-26, +5-26, +6-26, +7-26, +8-26, +9-26, +10-26, +11-26, +12-26, +13-26, +4-25, +5-25, +6-25, +7-25, +8-25, +9-25, +10-25, +11-25, +12-25, +13-25, +4-24, +5-24, +6-24, +7-24, +8-24, +9-24, +10-24, +11-24, +12-24, +13-24, +4-23, +5-23, +6-23, +7-23, +8-23, +9-23+10-23, +11-23, +12-23, +13-23, +4-22, +5-22, +6-22, +7-22, +8-22, +9-22, +10-22, +11-22, +12-22, +13-22, +4-21, +5-21, +6-21, +7-21, +8-21, +9-21, +10-21, +11-21, +12-21, +13-21, +4-20, +5-20, +6-20, +7-20, +8-20, +9-20, +10-20, +11-20, +12-20, +13-20, +4-19, +5-19, +6-19, +7-19, +8-19, +9-19, +10-19, +11-19, +12-19, +13-19, +4-18, +5-18, +6-18, +7-18, +8-18, +9-18, +10-18, or +11-18 relative to the 5′ splice site of intron 8 (e.g. the intron 8 set forth in SEQ ID NO:6), i.e. the antisense oligonucleotide has a sequence that is complementary to at least one the aforementioned regions.

In another example, the antisense oligonucleotide binds to a target region that spans or is within positions +45-70, +46-70, +47-70, +48-70, +49-70, +50-70, +51-70, +52-70, +53-70, +45-69, +46-69, +47-69, +48-69, +49-69, +50-69, +51-69, +52-69, +53-69, +45-68, +46-68, +47-68, +48-68, +49-68, +50-68, +51-68, +52-68, +53-68, +45-67, +46-67, +47-67, +48-67, +49-67, +50-67, +51-67, +52-67, +53-67, +45-66, +46-66, +47-66, +48-66, +49-66, +50-66, +51-66, +52-66, +53-66, +45-65, +46-65, +47-65, +48-65, +49-65, +50-65, +51-65, +52-65, +53-65, +45-64, +46-64, +47-64, +48-64, +49-64, +50-64, +51-64, +52-64, +53-64, +45-63, +46-63, +47-63, +48-63, +49-63, +50-63, +51-63, +52-63, +53-63, +45-62, +46-62, +47-62, +48-62, +49-62, +50-62, +51-62, +52-62, or +53-62 relative to the 5′ splice site of intron 8 (e.g. the intron 8 set forth in SEQ ID NO:6), i.e. the antisense oligonucleotide has a sequence that is complementary to at least one of the aforementioned regions.

In further embodiments, the antisense oligonucleotide binds to a target region that spans or is within positions +70-100, +70-99, +70-81, +70-80, +70-79, +71-81, +72-81, +73-81, +83-99, +83-98, +83-97, +84-99, +85-99, +86-99, +87-99, +84-97, +85-97, +86-97, or +87-97, relative to the 5′ splice site of intron 8, (e.g. the intron 8 set forth in SEQ ID NO:6), i.e. the antisense oligonucleotide has a sequence that is complementary to at least one of the aforementioned regions.

In some examples, the antisense oligonucleotide binds to, and thus comprises a sequence that is complementary to, positions +4-21, +5-22, +6-23, +7-24, +8-25, +9-26, +10-27, +11-28, +12-29, +13-30, +14-31, +15-32, +16-33, +17-34, +18-35, +19-36, +20-37, +21-38, +22-39, +23-40, +24-41, +25-42, +26-43, +27-44, +28-45, +29-46, +30-47, +31-48, +32-49, +33-50, +34-51, +35-52, +36-53, +37-54, +38-55, +39-56, +40-57, +41-58, +42-59, +43-60, +44-61, +45-62, +46-63, +47-64, +48-65, +49-66, +50-67, +51-68, +52-69, +53-70, +54-71, +55-72, +56-73, +57-74, +58-75, +59-76, +60-77, +61-78, or +62-79, +70-100, +70-99, +70-81, +70-80, +70-79, +71-81, +72-81, +73-81, +83-99, +83-98, +83-97, +84-99, +85-99, +86-99, +87-99, +84-97, +85-97, +86-97, or +87-97 relative to the 5′ splice site of intron 8. In some embodiments, the antisense oligonucleotide comprises a sequence having at least or about 70%, 80%, or 90% sequence identity to a sequence set forth in any one of SEQ ID NOs:83-143 (e.g., comprises 1, 2, 3, 4 or 5 nucleotide changes (e.g. substitutions, insertions or deletions) compared to the sequence set forth in any one of SEQ ID NOs:83-143), or a sequence having at least 8, 9, 10, 11, 12, 13, 14 or 15 contiguous nucleotides from a sequence set forth in any one of SEQ ID NOs:83-143. In a particular embodiment, the sequence has at least or about 70%, 80%, or 90% sequence identity to a sequence set forth in any one of SEQ ID NOs:86-96, or a sequence having at least 8, 9, 10, 11, 12, 13, 14 or 15 contiguous nucleotides. In a further example, the antisense oligonucleotide comprises the sequence set forth in any one of SEQ ID NOs:91-93, or a sequence comprising at least 8, 9, 10, 11, 12, 13, 14 or 15 contiguous nucleotides from a sequence set forth in any one of SEQ ID NOs:91-93.

In one embodiment, the antisense oligonucleotide binds to nucleotides within intron 9 in the intron-retaining SynGAP1 mRNA or pre-mRNA. Accordingly, in some examples, an antisense oligonucleotide of the present disclosure has a sequence of nucleobases that is complementary to a sequence of nucleotides within intron 9 of SynGAP1 pre-mRNA, such as within the intron 9 set forth as follows:

(SEQ ID NO: 7)
GTATGGCCCACACTCAGGCCCTCTTCTTCCCAAACCTGCCAGATGTCCA
CCCCAGACCCCAAGTCCACCCTTCCACAGCTTGATACTTCCTAACCCAG
AGTCCTAGGACTCCAGCCTCCAACACCTGATTCTGAAATTTCCCCAACC
CTGGCCACCCCCTTCCCTGCCCTTGGAAAGTGTGACCACACCCTCTTGT
GCCCCCACCCCCCAG

In some examples, the antisense oligonucleotide binds to (i.e. comprises a sequence that is complementary to) a target region in intron 9 of SynGAP1 spanning positions +4-200, +4-40, +80-120, or +160-200, relative to the 5′ splice site of intron 8. In particular embodiments, the antisense oligonucleotide binds to, or adjacent to, an ISS in intron 9. As determined herein, a putative ISS recognised by hnRNPA1 is at positions +104-108 relative to the 5′ splice site of intron 9 (bolded in SEQ ID NO:7, above), and putative ISS recognised by hnRNP I (PTB) are at positions +21-29 (multiple putative overlapping sites at +21-26, +22-26, +22-28, +24-29 and +25-29) and +190-195 relative to the 5′ splice site of intron 9 (underlined in SEQ ID NO:7, above). Thus, in some embodiments, the antisense oligonucleotide binds to, or adjacent to, nucleotides at positions +21-29, +104-108, or +190-195 relative to the 5′ splice site of intron 9. For example, the antisense oligonucleotide may bind to one or more of the nucleotides at position +10, +11, +12, +13, +14, +15, +16, +17, +18, +19, +20, +21, +22, +23, +24, +25, +26, +27, +28, +29, +30, +31, +32, +33, +34, +35 or +36 relative to the 5′ splice site of intron 9, or may bind to one or more of the nucleotides at position +90, +91, +92, +93, +94, +95, +96, +97, +98, +99, +100, +101, +102, +103, +104, +105, +106, +107, +108, +109, +110, +111, +112, +113, +114, +115, +116, +117, +118, +119 or +120 relative to the 5′ splice site of intron 9, or may bind to one or more of the nucleotides at position +175, +176, +177, +178, +179, +180, +181, +182, +183, +184, +185, +186, +187, +188, +189, +190, +191, +192, +193, +194, +195, +196, +197, +198, +199, +200, +201, +202, +203, +204, +205, +206, or +207 relative to the 5′ splice site of intron 9.

In one example, the antisense oligonucleotide binds to a target region in the intron-retaining SynGAP1 mRNA or pre-mRNA that spans or is within positions +10-41, +11-41, +12-41, +13-41, +14-41, +15-41, +16-41, +17-41, +18-41, +10-40, +11-40, +12-40, +13-40, +14-40, +15-40, +16-40, +17-40, +18-40, +10-39, +11-39, +12-39, +13-39, +14-39, +15-39, +16-39, +17-39, +18-39, +10-38, +11-38, +12-38, +13-38, +14-38, +15-38, +16-38, +17-38, +18-38, +10-37, +11-37, +12-37, +13-37, +14-37, +15-37, +16-37, +17-37, +18-37, +10-36, +11-36, +12-36, +13-36, +14-36, +15-36, +16-36, +17-36, +18-36, +10-35, +11-35, +12-35, +13-35, +14-35, +15-35, +16-35, +17-35, +18-35, +10-34, +11-34, +12-34, +13-34, +14-34, +15-34, +16-34, +17-34, +18-34, +10-33, +11-33, +12-33, +13-33, +14-33, +15-33, +16-33, +17-33, +18-33, +10-32, +11-32, +12-32, +13-32, +14-32, +15-32, +16-32, +17-32, +18-32, +10-31, +11-31, +12-31, +13-31, +14-31, +15-31, +16-31, +17-31, +18-31, +10-30, +11-30, +12-30, +13-30, +14-30, +15-30, +16-30, +17-30, or +18-30 relative to the 5′ splice site of intron 9 (e.g. the intron 9 set forth in SEQ ID NO:7), i.e. the antisense oligonucleotide has a sequence that is complementary to an aforementioned region. In some embodiments, the antisense oligonucleotide comprises a sequence having at least or about 70%, 80%, or 90% sequence identity to a sequence set forth in any one of SEQ ID NOs:144-167 (e.g., comprises 1, 2, 3, 4 or 5 nucleotide changes (e.g. substitutions, insertions or deletions) compared to the sequence set forth in any one of SEQ ID NOs:144-167), or a sequence having at least 8, 9, 10, 11, 12, 13, 14 or 15 contiguous nucleotides from a sequence set forth in any one of SEQ ID NOs:144-167. In a particular embodiment, the sequence has at least or about 70%, 80%, or 90% sequence identity to a sequence set forth in any one of SEQ ID NOs:152-155 (e.g., comprises 1, 2, 3, 4 or 5 nucleotide changes (e.g. substitutions, insertions or deletions) compared to the sequence set forth in any one of SEQ ID NOs:152-155), or a sequence having at least 8, 9, 10, 11, 12, 13, 14 or 15 contiguous nucleotides.

In another example, the antisense oligonucleotide binds to a target region in the intron-retaining SynGAP1 mRNA or pre-mRNA that spans or is within positions +87-120, +88-120, +89-120, +90-120, +91-120, +92-120, +93-120, +94-120, +95-120, +96-120, +97-120, +98-120, +87-119, +88-119, +89-119, +90-119, +91-119, +92-119, +93-119, +94-119, +95-119, +96-119, +97-119, +98-119, +87-118, +88-118, +89-118, +90-118, +91-118, +92-118, +93-118, +94-118, +95-118, +96-118, +97-118, +98-118, +87-117, +88-117, +89-117, +90-117, +91-117, +92-117, +93-117, +94-117, +95-117, +96-117, +97-117, +98-117, +87-116, +88-116, +89-116, +90-116, +91-116, +92-116, +93-116, +94-116, +95-116, +96-116, +97-116, +98-116, +87-115, +88-115, +89-115, +90-115, +91-115, +92-115, +93-115, +94-115, +95-115, +96-115, +97-115, +98-115, +87-114, +88-114, +89-114, +90-114, +91-114, +92-114, +93-114, +94-114, +95-114, +96-114, +97-114, +98-114, +87-113, +88-113, +89-113, +90-113, +91-113, +92-113, +93-113, +94-113, +95-113, +96-113, +97-113, +98-113, +87-112, +88-112, +89-112, +90-112, +91-112, +92-112, +93-112, +94-112, +95-112, +96-112, +97-112, +98-112, +87-111, +88-111, +89-111, +90-111, +91-111, +92-111, +93-111, +94-111, +95-111, +96-111, +97-111, +98-111, +87-110, +88-110, +89-110, +90-110, +91-110, +92-110, +93-110, +94-110, +95-110, +96-110, +97-110, or +98-110, relative to the 5′ splice site of intron 9 (e.g. the intron 9 set forth in SEQ ID NO:7), i.e. the antisense oligonucleotide has a sequence that is complementary to an aforementioned region. In some embodiments, the antisense oligonucleotide comprises a sequence having at least or about 70%, 80%, or 90% sequence identity to a sequence set forth in any one of SEQ ID NOs:168-189 (e.g., comprises 1, 2, 3, 4 or 5 nucleotide changes (e.g. substitutions, insertions or deletions) compared to the sequence set forth in any one of SEQ ID NOs:168-189), or a sequence having at least 8, 9, 10, 11, 12, 13, 14 or 15 contiguous nucleotides from a sequence set forth in any one of SEQ ID NOs: 166-187. In a particular embodiment, the sequence has at least or about 70%, 80%, or 90% sequence identity to a sequence set forth in any one of SEQ ID NOs:170-172 (e.g., comprises 1, 2, 3, 4 or 5 nucleotide changes (e.g. substitutions, insertions or deletions) compared to the sequence set forth in any one of SEQ ID NOs: 170-172), or a sequence having at least 8, 9, 10, 11, 12, 13, 14 or 15 contiguous nucleotides from a sequence set forth in any one of SEQ ID NOs: 170-172. In another embodiment, the sequence has at least or about 70%, 80%, or 90% sequence identity to a sequence set forth in any one of SEQ ID NOs:179-181 and 183 (e.g., comprises 1, 2, 3, 4 or 5 nucleotide changes (e.g. substitutions, insertions or deletions) compared to the sequence set forth in any one of SEQ ID NOs: 179-181 and 183), or a sequence having at least 8, 9, 10, 11, 12, 13, 14 or 15 contiguous nucleotides from a sequence set forth in any one of SEQ ID NOs: 177-179 and 181.

In a further examples, the antisense oligonucleotide binds to a target region in the intron-retaining SynGAP1 mRNA or pre-mRNA that spans or is within positions +175-205, +176-205, +177-205, +178-205, +179-205, +180-205, +181-205, +182-205, +183-205, +184-205, +185-205, +175-204, +176-204, +177-204, +178-204, +179-204, +180-204, +181-204, +182-204, +183-204, +184-204, +185-204, +175-203, +176-203, +177-203, +178-203, +179-203, +180-203, +181-203, +182-203, +183-203, +184-203, +185-203, +175-202, +176-202, +177-202, +178-202, +179-202, +180-202, +181-202, +182-202, +183-202, +184-202, +185-202, +175-201, +176-201, +177-201, +178-201, +179-201, +180-201, +181-201, +182-201, +183-201, +184-201, +185-201, +175-200, +176-200, +177-200, +178-200, +179-200, +180-200, +181-200, +182-200, +183-200, +184-200, +185-200, +175-199, +176-199, +177-199, +178-199, +179-199, +180-199, +181-199, +182-199, +183-199, +184-199, +185-199, +175-198, +176-198, +177-198, +178-198, +179-198, +180-198, +181-198, +182-198, +183-198, +184-198, +185-198, +175-197, +176-197, +177-197, +178-197, +179-197, +180-197, +181-197, +182-197, +183-197, +184-197, +185-197, +175-196, +176-196, +177-196, +178-196, +179-196, +180-196, +181-196, +182-196, +183-196, +184-196, +185-196, +175-195, +176-195, +177-195, +178-195, +179-195, +180-195, +181-195, +182-195, +183-195, +184-195, or +185-195, relative to the 5′ splice site of intron 9 (e.g. the intron 9 set forth in SEQ ID NO:7), i.e. the antisense oligonucleotide has a sequence that is complementary to an aforementioned region.

In another embodiment, the antisense oligonucleotide binds to nucleotides within intron 5 in the intron-retaining SynGAP1 mRNA or pre-mRNA. Accordingly, in some examples, an antisense oligonucleotide of the present disclosure has a sequence of nucleobases that is complementary to a sequence of nucleotides within intron 5 of an intron-retaining SynGAP1 mRNA or pre-mRNA, such as within the intron 5 set forth as follows:

(SEQ ID NO: 5)
GTACAGGGGCTGGAGCATGTGGGATGAGATTGATGTAATGTAGGGTCTC
CTGTGTGAGATGCAGAGGGAGGGGGTTATCTGTGTGCAAAGGTTGAAGG
ATTCAACTCAAGTTGGTTGGGGGATGTCATGGCACAGGGGACAGAACAG
AAAAGAACTAGAATAGGGATCTGTGAGCAGCAGGAGAGGGGTAGGGTGG
CAGAGAGAAGACAGACAGACAGGCTGGAAAGGGAATGAAGGTGAAGCCA
AGGAGGGACTCCTCAGGGACTCCTCAGGCCAAGAAGGATGGGCTCTAGC
CCAGGATCAAAGGAGCTGTACAGGAGGAGAGTGACCCTGGAGGAATGTT
TAAGGAATGCAGGGAAGGGGTTGGTAGGTGAGTGAGCAATAGGCTGTAG
GTGGAAGGGTGTCAGGGAAGGTCAGGAAATACAGGGGCAGCAGGTTGGA
GTGGGGCTGGGGGTGGCTGAATGAATGGATGATGGCTAGGGCTCAAGGA
CCTCATCAGTGAGGGAAGAGACAGTATAGAGCATGGCAGAGAAGGGGAG
GCTGGGACAGGTGTGCAGGGTGACAGAATGGGAAGCAACCCATGGACTG
AGGCATGAAGAAGCAGCCAGCGGAGAAGTCCAGAAGGCACTGTCCCTGA
GACCAGGCTGAAGGAGACCTCCACTGTTTGCCTTTGTTGCCTGCCATTT
GGGGTTCCTCTCTGGGTTTCCCCCTCACCCAGTCACTCCCCAGGGAGAA
CCATGCCCTCCCTTTCCCCCATGTCTGGCCACCCCCAGGATTGGGCAGG
TAGGGAGGTTGGGATAAAGTGAGTCACACCTTTCCCTGCCCCCCTCCCA
TGTTGCCAGAGCTGGATTTGGGGCCGGCAGGGGGTGAGGGCATGGTATT
CCTGGCCGCGGGGGCGGGGGGGGGGGTCCGGGGGCCGGGGGAGCGTCGC
GCTGACGGCAGCCAGAGCCTGCGATGACGGGGCTGCTATAAATAACTTC
TTGGAGGCTCCCACACCCAAGCTCCCCTCCCGCTTTCCCACTGCTCTCT
ACTCTTCATCCCCTGCCCATCTCCATACCGCTTTTGTATTGCTATCCTA
CCCCTCATTATTCCATGCCCCTAGCCCCCTTTATCTTCTGCCCTCCTGC
AGTGATTTTTTTGCATTCCATCCCCTCTTAGCCCTCACCTCGGTTCTCC
CGGCCATCTCTCCAGTTGGCCTTCCTCCTCTTCTCCTGTCCTCTGTCTT
GCTGCACATACCTTTGTCTCCCCCTTTCTTCTTCTTGCCCTACCTCCTC
TTCTTCCCTAGTCCGTGTATTCTGTCTTTTATCCTCTTTGAGCTCTTTT
CTGCCCACAGCTTTCTCCTATTTCTTATGCTTTTCCCTCACTCTTTCCC
CTGCTTCTGCTAAAACTTGTCCTCTTATGCTGTGTTCATTCATTCTTTG
AATCATTAAATGTTTATCAGGCACTAGCCGTGTGCCAGGCCCAGGCTAG
ACATATCTCTTCTCTGTGCCTTCACTTCTTTACTTCCACTTTTTCCTTT
ATACTGAGGCTCTGGTTTCTGGGGTTACCTGGAGGTACTACCTAGAAGT
GCCCCAGGCCCACTTTGTTCTCTCCTTTTTTTTTTTTCTTTTCTGCCAT
GGTCCATTTCTGGGTTGAGATATTTCTAGATGTCCCCAGTCCTCGCAAT
CCCTTAGGTGTGAGATGGTGGGAGTTTCTTTTTTTTCCTTTTTTTTTTT
TTAAATAGAAATAGGGTCTCACTGTGTTGCCCAGACTGGTCTTGAACTC
CTGGGCTCAAGTGACCCTCCCACCTCGGCCTTTGAAATGTTGGGATTAC
AGGTGTGAGCCACCAGGCCCAGGTGGAGCAGGGGAGTTCCTTAAAGGAT
TCTGATTTTTCTCACATCCCTCACGTCCTTCCTGATAGGCAGGGTTTCT
TTCTGTGTCTGTTTGGGAAGGGTGTTCAGGGGGCCTTCTCTCCAAGTCT
CCATCCTGGAACAGACTGATGATGCAGGGTACCTATGTGTCTAAGAAGA
GTAGGGGGGCCGGGCGCGGTGGCTCATGCCTGTAATCCCAGCACTTTGG
GAGGCTGAATCACTTGAGGTCAGGAGTTTGAGACCAGCCTGACCAACAG
GGTGAAACCCCGTCTCAGCTAAAAATACAAAAAAAAAAAAGAAAAAAAA
ATTAGCTGGGTGTGCTGAGGCAGGAGAGACGCTTGAGCCCAGGAGGCAG
AAGTTGCAGCAAGCCGAGATCACACCACTGTACTCCAGCCTGGGCGACA
GAGCAAGACTGTCTCAAAAAAAAAAAAAAAAAAAAAAAAGGAAGAGTGG
GAAGCCCTGATCCCTTCCTCTCCTGAACCTCCTGCCTGCCAG

In another embodiment, the antisense oligonucleotide binds to nucleotides within intron 12 in the intron-retaining SynGAP1 mRNA or pre-mRNA. Accordingly, in some examples, an antisense oligonucleotide of the present disclosure has a sequence of nucleobases that is complementary to a sequence of nucleotides within intron 12 of an intron-retaining SynGAP1 mRNA or pre-mRNA, such as within the intron 12 set forth as follows:

(SEQ ID NO: 8)
GTCAGCAGATCCCCTCTTTGCCCTATCCCCAGATGGCTCCAGAGGTTCC
TGGAGCCTGAGAAACTACCCTTTGAAGATTTTTTTTCTCCCCTTGTTTC
TCGAGGTGTCACCACTACTATCCCAACTCAGGCCCCCTCCACCTGCACC
CTCAGAGGCCCTCTTAGAGCTGGGCACTGAGCCCCCAGGTAACAGCCTC
ACCCTTCCAG

In a further embodiment, the antisense oligonucleotide binds to nucleotides within intron 13 in the intron-retaining SynGAP1 mRNA or pre-mRNA. Accordingly, in some examples, an antisense oligonucleotide of the present disclosure has a sequence of nucleobases that is complementary to a sequence of nucleotides within intron 13 of an intron-retaining SynGAP1 mRNA or pre-mRNA, such as within the intron 13 set forth as follows:

(SEQ ID NO: 9)
GTCAGCAGATCCCCTCTTTGCCCTATCCCCAGATGGCTCCAGAGGTTCC
TGGAGCCTGAGAAACTACCCTTTGAAGATTTTTTTTCTCCCCTTGTTTC
TCGAGGTGTCACCACTACTATCCCAACTCAGGCCCCCTCCACCTGCACC
CTCAGAGGCCCTCTTAGAGCTGGGCACTGAGCCCCCAGGTAACAGCCTC
ACCCTTCCAG

In another embodiment, the antisense oligonucleotide binds to nucleotides within intron 14 in the intron-retaining SynGAP1 mRNA or pre-mRNA. Accordingly, in some examples, an antisense oligonucleotide of the present disclosure has a sequence of nucleobases that is complementary to a sequence of nucleotides within intron 14 of an intron-retaining SynGAP1 mRNA or pre-mRNA, such as within the intron 14 set forth as follows:

(SEQ ID NO: 10)
GTGAGGGGCTCTCCCCTCCCCCGCCCTCCTCTCCTCTCCTGTCTGTTCC
CTCTCCCACTCCACTGGCCTTCGCCCTACTCCTCTCCTCTCCTCCTCCA
TGGACCTCATCTCCTCCATATGTGCCCAGCCCTGCCCCCATCCCTTCTC
TTGCTGCCCCCATCTCCCCTCCTCTAGGCCTCACCCCCTTCCCGGAGGG
GCCCTGTCCTTTCCCTTTACTCACCTGTCCCCTCCCATCCTCCCTGCCT
GCCCTCTTCAGGGCTGCCACCGCTAGCTCTCAGCCCTTCCCTCTGGGTC
CCACTTTTCACCCCAAGGCCTGTGCCAGACCACAGCAAGGTTCAATTGC
TAGGAGCCCTGACCTTACCTTCTGCTTGTGTGCCCCCTTCCCTTCTGAC
AG

The antisense oligonucleotides of the present disclosure can enhance splicing such that the amount or level of the fully-spliced SynGAP1 mRNA or the amount or level of SynGAP1 protein in the cell or population of cells that is contacted with the antisense oligonucleotide is increased by at least or about 20%, 30%, 40%, 50%, 60%, 70%, 80%, 90%, 100%, 110%, 120%, 130%, 140%, 150%, 200%, 250%, 300%, 350%, 400%, 450%, 500%, 600%, 700% or more compared to the amount or level of a fully-spliced SynGAP1 mRNA or of a SynGAP1 protein in a cell or population of cells that has not been contacted with an antisense oligonucleotide of the present disclosure. Thus, in some instances, the amount or level of a fully-spliced SynGap1 mRNA or of a SynGAP1 protein in the cell or population of cells following exposure to an antisense oligonucleotide of the present disclosure is 1.2×, 1.3×, 1.4×, 1.5×, 1.6×, 1.7×, 1.8×, 1.9×, 2×, 2.1×, 2.2×, 2.3×, 2.4×, 2.5×, 3×, 3.5×, 4×, 4.5×, 5×, 6×, 7× or more compared to the amount or level of a fully-spliced SynGAP1 mRNA or of a SynGAP1 protein in a cell or population of cells that has not been exposed to an antisense oligonucleotide of the present disclosure. In some examples, the fully-spliced SynGAP1 mRNA is that described as NCBI Reference Sequence: NM_006772.3 (SEQ ID NO:1), and/or the SynGAP1 protein is that described as NCBI Reference Sequence: NP_006763.3 (SEQ ID NO:3). In other examples, the fully-spliced SynGAP1 mRNA is that described as NCBI Reference Sequence: NM_001130066.2 (SEQ ID NO:2), and/or the SynGAP1 protein is that described as NCBI Reference Sequence: NP_001123538.1 (SEQ ID NO:4).

The antisense oligonucleotides of the present disclosure are typically 8 to 50, nucleobases in length, such as 8 to 50, 8 to 40, 8 to 35, 8 to 30, 8 to 25, 8 to 20, 8 to 15, 9 to 50, 9 to 40, 9 to 35, 9 to 30, 9 to 25, 9 to 20, 9 to 15, 10 to 50, 10 to 40, 10 to 35, 10 to 30, 10 to 25, 10 to 20, 10 to 15, 11 to 50, 11 to 40, 11 to 35, 11 to 30, 11 to 25, 11 to 20, 11 to 15, 12 to 50, 12 to 40, 12 to 35, 12 to 30, 12 to 25, 12 to 20, or 12 to 15 nucleobases in length. Thus, in particular examples, the antisense oligonucleotides are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

The antisense oligonucleotides may be 100% complementary across their entire length to a target region of an intron-retaining SynGAP1 mRNA or pre-mRNA or may be less than 100% complementary. Typically, the antisense oligonucleotides are at least 70%, 80%, 85%, 86%, 87%, 88%, 89%, 90%, 91%, 92%, 93%, 94%, 95%, 96%, 97%, 98%, or 99% complementary to a target region of an intron-retaining SynGAP1 mRNA or pre-mRNA, such as a region identified above in intron 5, 8, 9, 12, 13, or 14. The antisense oligonucleotides may contain, for example, at least 8, at least 9, at least 10, at least 11, at least 12, at least 13, at least 14, at least 15, at least 16, at least 17, at least 18, at least 19, or at least 20 contiguous nucleobases that are complementary to a target region in an intron-retaining SynGAP1 mRNA or pre-mRNA. In instances where the antisense oligonucleotides are not 100% complementary, the mismatched or non-complementary nucleobase(s) can be clustered or interspersed with complementary nucleobases and need not be contiguous to each other. The non-complementary nucleobase(s) may be located at the 5′ end and/or 3′ end of the antisense compound. Alternatively, the non-complementary nucleobase(s) can be at an internal position of the antisense oligonucleotide. When two or more non-complementary nucleobases are present, they can be either contiguous or non-contiguous.

In particular embodiments, antisense oligonucleotides of the present disclosure are up to 10, 12, 13, 14, 15, 16, 17, 18, 19, 20, 21, 22, 23, 24, 25, 26, 27, 28, 29 or 30 nucleobases in length and comprise no more than 6, 5, 4, 3, 2, or 1 non-complementary nucleobase(s) relative to a target region in an intron-retaining SynGAP1 mRNA or pre-mRNA.

The antisense oligonucleotides of the present disclosure can be produced using any method known in the art. Typically, the antisense oligonucleotides are produced using chemical synthesis methods. While the antisense oligonucleotides can be unmodified, more typically the antisense oligonucleotides of the present disclosure contain one or more modifications. These modifications can function to, for example, increase stability of the antisense oligonucleotide (e.g. increase resistance of the antisense oligonucleotide to degradation by nucleases), increase affinity of the antisense oligonucleotide to the target mRNA or pre-mRNA, increase steric hindrance by the antisense oligonucleotide, increase RNase H activity, and/or improve intracellular uptake. Exemplary modifications that are well known to those skilled in the art include, but are not limited to, modification of the nucleobase, modification of the backbone phosphate linkages (e.g. phosphodiester, phosphoramidate, or phosphorothioate (PS) modification), modifications of the ribose sugar (e.g. 2′-O-methyl (20Me), 2′-O-methoxy-ethyl (MOE), locked nucleic acids (LNA), 2′-fluoro and S-constrained-ethyl (cEt) modifications) and other modifications such as replacement of the entire sugar phosphate backbone with polyamide linkages to produce peptide nucleic acids (PNA) and the use of a morpholine ring instead of the ribose ring and phosphoroamidate intersubunit linkages to produce phosphorodiamidate morpholino oligomers (PMO) (broadly reviewed in, for example, Sardone et al. (2017) Molecules 22(4): 563 Evers et al. (2015) Adv Drug Del Rev 87:90-103; Kole et al. (2012) Nat Rev Drug Discov. 11(2): 125-140).

In particular embodiments, the antisense oligonucleotides of the present disclosure contain one or more modified nucleobases. These can function to, for example, increase stability or binding affinity of the antisense oligonucleotide. Exemplary modified nucleobases include, but are not limited to, N⁶-methyladenine, N²-methylguanine, hypoxanthine, 7-methylguanine, 5-methylcytosine, 5-hydroxymethylcytosine, pseudouracil, 4-thiouracil, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-a minomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N⁴-ethylcytosine, or derivatives thereof; N²-cyclopentylguanine (cPent-G), N2-cyclopentyl-2-aminopurine (cPent-AP), and N²-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, I-deoxy-2-O-methylribose; or pyrrolidine derivatives in which the ring oxygen has been replaced with nitrogen (azaribose)). In particular embodiments, the antisense oligonucleotides contain one or more modified nucleobases that increase the binding affinity of the antisense oligonucleotide to the SynGAP1 mRNA or pre-mRNA, such as 5-methylcytosine (5-me-C), 5-substituted pyrimidines, 6-azapyrimidines and N-2, N-6 and 0-6 substituted purines, including 2 aminopropyladenine, 5-propynyluracil and 5-propynylcytosine.

The antisense oligonucleotides of the present disclosure may comprise modified sugar moieties. Exemplary sugar moiety modifications include 2′-O-methyl (20Me), 2′-O-methoxy-ethyl (MOE), locked nucleic acids (LNA), 2′-fluoro and S-constrained-ethyl (cEt) modifications.

In particular embodiments, the backbones of the antisense oligonucleotides of the present disclosure comprise phosphorothioates, chiral phosphorothioates, phosphorodithioates, phosphotriesters, aminoalkylphosphotriesters, methyl or other alkyl phosphonates comprising 3′alkylene phosphonates or chiral phosphonates, phosphinates, phosphoramidates comprising 3′-amino phosphoramidate and aminoalkylphosphoramidates, thionophosphoramidates, thionoalkylphosphonates, thionoalkylphosphotriesters, or boranophosphates. In other embodiments, the backbone has no phosphorus atom. Exemplary oligonucleotide backbones that do not include a phosphorus atom include those that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside; see e.g. U.S. Pat. Nos. 5,698,685, 5,217,866, 5,142,047, 5,034,506, 5,166,315, 5,185,444, 5,521,063, 5,506,337, 8,076,476, 8,299,206 and 7,943,762); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In one example, the antisense oligonucleotides of the present disclosure are a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone (see e.g. U.S. Pat. Nos. 5,539,082; 5,714,331; and 5,719,262).

In particular embodiments, the antisense oligonucleotides of the present invention are partially or completely resistant to RNase H. Such antisense oligonucleotides can include 2′-O-methyl derivatives, and/or phosphorothioate backbones, both of which are resistant to nuclease degradation. In further examples, the antisense oligonucleotides do not activate RNase H, typically by virtue of the presence of one or more structural modifications that sterically hinders or prevent binding of RNase H to a duplex molecule containing the antisense oligonucleotide and the KCNT1 mRNA or pre-mRNA. For example, such antisense oligonucleotides include those where at least one, or all, of the inter-nucleotide bridging phosphate residues are modified phosphates, such as methyl phosphonates, methyl phosphorothioates, phosphoromorpholidates, phosphoropiperazidates and phosphoramidates. For example, every other one of the internucleotide bridging phosphate residues may be modified as described. In another non-limiting example, such antisense molecules are molecules wherein at least one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl).

In other examples, the antisense oligonucleotides of the present disclosure activate RNase H when they form a DNA-RNA duplex with the SynGAP1 mRNA or pre-mRNA. Exemplary of such antisense oligonucleotides are gapmers, which are chimeric molecules containing at least one region modified so as to confer increased resistance to nuclease degradation, increased cellular uptake, increased binding affinity for the target nucleic acid, and a second region that serves as a substrate for RNase H. Gapmers have an internal region having a plurality of nucleotides that support RNase H cleavage. This internal region is positioned between external regions having a plurality of nucleotides that are chemically distinct from the nucleosides of the internal region, and which serve to, for example, increase stability of the antisense oligonucleotide and protect it from nuclease degradation. In certain embodiments, the external regions of the gapmer contain β-D-ribonucleosides, β-D-deoxyribonucleosides, 2′-modified nucleosides (e.g. 2′-MOE, and 2′-O—CH₃, among others), bridged nucleic acids (BNAs), or locked nucleic acids (LNAs).

The antisense oligonucleotides of the present disclosure may also be linked to one or more one or more moieties that enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, carbohydrates, phospholipids, biotin, phenazine, folate, phenanthridine, anthraquinone, acridine, fluoresceins, rhodamines, coumarins, and various dyes.

In particular embodiments, the antisense oligonucleotides are linked to a cell-penetrating peptide (CPP) that is effective to enhance transport of the compound into cells. The transport moiety can be attached to either terminus of the antisense oligonucleotide, resulting in increased penetration of the antisense oligonucleotides into cells and macromolecular translocation within multiple tissues in vivo upon systemic administration. In one embodiment, the cell-penetrating peptide is an arginine-rich peptide transporter. Antisense oligonucleotides linked with arginine-rich CPPs were able to cross the blood-brain barrier and were widely distributed throughout the brain of wild-type mice following systemic delivery (Du et al. Hum. Mol. Genet., 20 (2011), pp. 3151-3160). In another embodiment, the cell-penetrating peptide may be Penetratin or the Tat peptide. These peptides are well known in the art and are disclosed, for example, in US Publication No. 20100016215. The transport moieties described above have been shown to greatly enhance cell entry of attached oligomers, relative to uptake of the oligomer in the absence of the attached transport moiety. For example, antisense oligonucleotides linked with arginine-rich CPPs were able to cross the blood-brain barrier and were widely distributed throughout the brain of wild-type mice following systemic delivery (Du et al. Hum. Mol. Genet., 20 (2011), pp. 3151-3160). Uptake may be enhanced at least ten-fold, or at least twenty-fold, relative to the unconjugated compound. In other examples, the antisense oligonucleotide is coupled to a dopamine reuptake inhibitor (DRI), a selective serotonin reuptake inhibitor (SSRI), a noradrenaline reuptake inhibitor (NRI), a norepinephrine-dopamine reuptake inhibitor (NDRI), or a serotonin-norepinephrine-dopamine reuptake inhibitor (SNDRI), as described in, e.g., U.S. Pat. No. 9,193,969. In further examples, the antisense oligonucleotides are conjugated to peptides collectively known as “angiopeps” which are capable of crossing the blood-brain barrier by receptor-mediated transcytosis using the low-density lipoprotein receptor-related protein-1 (LRP-1), and which allow the delivery of systemically administered antisense-peptide conjugates to the brain (see e.g. WO200979790).

The antisense oligonucleotides can also be modified to have one or more stabilizing groups that are generally attached to one or both termini to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps.

### Assessment of the Antisense Oligonucleotides

Antisense oligonucleotides of the present disclosure can be designed rationally, so as to target a specific region or site in an intron (e.g. an ISS, a G-quadruplex or a region with a propensity for secondary structure) and/or by methods such as antisense microwalk or tiling that cover the whole intron or just a region of an intron. For example, the antisense oligonucleotides used in the antisense walk can be tiled every 1, 2, 3, 4, 5, 6, 7, 8, 9 or 10 nucleotides from approximately 100 nucleotides upstream of the 5 ‘ splice site of the retained intron (e.g. in the preceding exon) to approximately 100 nucleotides downstream of the 5’ splice site and/or from approximately 100 nucleotides upstream of the 3 ‘ splice site of the retained intron to approximately 100 nucleotides downstream of the 3’ splice site of the target/retained intron (e.g. in the following exon). The activity of these antisense oligonucleotides can then be assessed and confirmed using various techniques known in the art. For example, the ability of the antisense oligonucleotides to enhance splicing, and thereby increase production of fully-spliced SynGAP1 mRNA and/or SynGAP1 protein can be assessed using in vitro assays to confirm that the antisense oligonucleotides are suitable for use in the methods of the present disclosure. Mouse models can be used to not only assess the ability of the antisense oligonucleotides to increase the level or amount of fully-spliced SynGAP1 mRNA and/or SynGAP1 protein in vivo, but to also ameliorate symptoms associated with heterozygous loss-of-function SYNGAP1 mutations.

In one example, cells such as mammalian neuronal cells (e.g. ARPE19, SH-SY5Y or SK-N-AS cells) are transfected with an antisense oligonucleotide of the present disclosure. The levels of fully-spliced SynGAP1 mRNA and intron-retaining SynGAP1 mRNA can be assessed using qRT-PCR or Northern blot as is well known in the art. The level SynGAP1 protein can also be assessed, such as by Western blot on total cell lysates or fractions.

The levels of fully-spliced SynGAP1 mRNA, intron-retaining SynGAP1 mRNA and/or SynGAP1 protein observed when cells are exposed to an antisense oligonucleotide of the present disclosure are compared to the respective levels observed when cells are exposed with a negative control antisense oligonucleotide, so as to determine the change resulting from the antisense oligonucleotide of the present disclosure. Typically, the level of fully-spliced SynGAP1 mRNA and/or SynGAP1 protein is increased by at least or about 20%, 25%, 30%, 35%, 40%, 45%, 50%, 55%, 60%, 65%, 70%, 75%, 80%, 85%, 90%, 100%, 110%, 120%, 125%, 30%, 135%, 140%, 145%, 150%, 155%, 160%, 165%, 170%, 175%, 180%, 185%, 190%, 200%, 250%, 300%, 350%, 400% or more. In such instances, the antisense oligonucleotides of the present disclosure can be used for treating a disease or condition associated with a heterozygous loss-of-function mutation in SYNGAP1.

Mouse models can also be used to assess and confirm the activity of the antisense oligonucleotides of the present disclosure. For example, an antisense oligonucleotide can be administered to a heterozygous SYNGAP1 knockout mouse, which displays physical and behavioural traits similar to those observed in patients with SYNGAP1-related intellectual disability (see e.g. Nakajima et al. 2019, Neuropsychopharmacol Rep. 39(3):223-237; Guo et al., 2009, Neuropsychopharmacol. 2009 34(7):1659-72). The ability of the antisense oligonucleotides of the present disclosure to enhance splicing, increase the levels of fully-spliced SynGAP1 mRNA and/or SynGAP1 protein, and/or ameliorate any symptoms associated with the SYNGAP1 mutation can then be assessed. In a particular example, SynGAP1 mRNA and/or protein levels in the brain, and in particular the neurons, are assessed. The levels of fully-spliced SynGAP1 mRNA, intron-retaining SynGAP1 mRNA and/or SynGAP1 protein following administration of an antisense oligonucleotide of the present disclosure are compared to the respective levels observed when a negative control antisense oligonucleotide is administered to the mice, so as to determine the change resulting from the antisense oligonucleotide of the present disclosure. Typically, the level of fully-spliced SynGAP1 mRNA and/or SynGAP1 protein is increased by at least or about 20%, 25%, 30%, 35%, 40%, 45%, 50%, 55%, 60%, 65%, 70%, 75%, 80%, 85%, 90%, 100%, 110%, 120%, 125%, 30%, 135%, 140%, 145%, 150%, 155%, 160%, 165%, 170%, 175%, 180%, 185%, 190%, 200%, 250%, 300%, 350%, 400% or more. In another example, the effect of administration of an antisense oligonucleotide of the present disclosure on the physical and/or behavioural traits of the mice is assessed. For example, behavioural and electrophysiological measures of memory and seizure in the mice can be assessed as described by Creson et al. (eLife 2019; 8:e46752).

### Compositions

The present disclosure provides compositions comprising the antisense oligonucleotides described above and herein. In particular examples, provided are pharmaceutical compositions comprising the antisense oligonucleotides and a pharmaceutically acceptable carrier. The compositions can also comprise additional ingredients such as carriers, diluents, stabilizers and excipients. The compositions can include one or more than one antisense oligonucleotide (e.g. two or more antisense oligonucleotides targeting the same or different introns), and further may comprise one or more other therapeutic agents.

The carriers, diluents, stabilizers and excipients can include buffers such as phosphate, citrate, or other organic acids; antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

The antisense oligonucleotides may also be formulated in compositions with liposomes, nanoparticles, microparticles, microspheres, lipid particles, vesicles, and the like, for the introduction of the antisense oligonucleotides of the present disclosure into cells. In embodiments, a penetration enhancer is included to effect the efficient delivery of the antisense oligonucleotide, e.g., to aid diffusion across cell membranes and/or enhance the permeability of a lipophilic drug. In some embodiments, the penetration enhancer is a surfactant, fatty acid, bile salt, chelating agent, or non-chelating nonsurfactant. Thus, provided are liposomes, nanoparticles, microparticles, microspheres, lipid particles, and vesicles comprising an antisense oligonucleotide of the present disclosure.

In embodiments, the antisense oligonucleotide is formulated in the context of a viral vector (e.g. adeno-associated viral (AAV) vector) where the vector comprises a genome that encodes an antisense oligonucleotide of the present disclosure.

Compositions comprising the antisense oligonucleotides encompass compositions comprising any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure also provides pharmaceutically acceptable salts of the antisense oligonucleotides described herein and other bio equivalents. Suitable pharmaceutically acceptable salts include, but are not limited to, sodium and potassium salts.

### Methods

The antisense oligonucleotides described above and herein can be used to increase levels of fully-spliced SynGAP1 mRNA and/or SynGAP1 protein in a cell (e.g. a neuronal cell) or in a subject. Consequently, the antisense oligonucleotides described above and herein can be used to treat a disorder associated with a heterozygous loss-of-function mutation in SYNAGP1, e.g. mental retardation, autosomal dominant 5 (MRD5; sometimes referred to as SYNGAP1-related intellectual disability) or autism or intellectual disability associated with a heterozygous loss-of-function mutation in SYNAGP1. The methods of the present disclosure therefore include a step of contacting a cell to an antisense oligonucleotide of the present disclosure, and/or administering an antisense oligonucleotide of the present disclosure to a subject. As would be appreciated, the phrase “administering an antisense oligonucleotide” and grammatical variations thereof encompasses embodiments where a composition comprising the antisense oligonucleotide is administered to subject, and embodiments where a composition comprising an agent that encodes the antisense oligonucleotide (e.g. a viral vector) is administered to subject. In the latter embodiment, it is understood that the antisense oligonucleotide is expressed in vivo, thereby effecting administration of the antisense oligonucleotide to the subject.

In some examples, the subject presenting with a disease or condition that may be associated with a heterozygous loss-of-function mutation in SYNAGP1 is genotyped to confirm the presence of a known heterozygous loss-of-function mutation in SYNAGP1 prior to administration of the antisense oligonucleotides and compositions thereof. For example, whole exome sequencing can be performed on the subject. Known heterozygous loss-of-function mutations in SYNAGP1 may include, but are not limited to, those described in Vlaskamp et al. (Neurology, 2019, 92(2):e96-e97). In other examples, the subject is first genotyped to identify the presence of a mutation in SYNGAP1 and this mutation is then confirmed to be a loss-of-function mutation, e.g. by assessing the levels of SynGAP1 mRNA or protein.

The precise amount or dose of the antisense oligonucleotide administered to the subject depends on, for example, the efficacy of the antisense oligonucleotide, the presence of other moieties (e.g. CCPs), the route of administration, the number of dosages administered, and other considerations, such as the weight, age and general state of the subject. Particular dosages and administration protocols can be empirically determined or extrapolated from, for example, studies in animal models or previous studies in humans, or may be otherwise determined by those skilled in the art using standard procedures.

The antisense oligonucleotides can be administered by any method and route understood to be suitable by a skilled artisan. Typically, the antisense oligonucleotides are administered parenterally, such as by subcutaneous administration, intravenous administration, intramuscular administration, intraarterial administration, intraperitoneal administration, or intracranial administration, e.g., intrathecal or intracerebroventricular administration. In other embodiments, the antisense oligonucleotides are delivered intranasally. Administration of the antisense oligonucleotides in the methods described herein preferably results in delivery of the antisense oligonucleotides to the central nervous system. In particular embodiments, the antisense oligonucleotides are administered intrathecally or by intracerebroventricular administration. The methods of the present invention can involve any combination of any two or more routes.

The antisense oligonucleotides can be administered to a subject one time or more than one time, including 2, 3, 4, 5 or more times. Where the antisense oligonucleotides are administered more than one time, the time between dosage administration can be, for example, 1, 2, 3, 4, 5, 6, 7, 8, 9, 10, 11, 12 or more weeks, or 1, 2, 3, 4, 5, 6, 7, 8, 9, 10, 11, 12 or more months. Selecting an optimal protocol is well within the level of skill of the skilled artisan and may depend on, for example, the half-life of the antisense oligonucleotide and the severity of the condition. In a particular embodiment, the antisense oligonucleotides are administered about every 3 months.

The antisense oligonucleotides, if desired, can be presented in a package, in a kit or dispenser device, such as a syringe with a needle, or a vial and a syringe with a needle, which can contain one or more unit dosage forms. The kit or dispenser device can be accompanied by instructions for administration.

In order that the invention may be readily understood and put into practical effect, particular preferred embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### Example 1

### Materials and Methods

Cell culture: The ARPE19 cell line used in this study was obtained from ATCC, USA. The cell lines SH-SY5Y and SK-N-AS were obtained from ECACC. The human brain RNA was purchased from Ambion, USA and from Takara-Bio, USA. The cells were grown in Dulbeccos modified Eagle's medium (DMEM) supplemented with 10% FBS. The cells were maintained at 37° C. and 5% CO₂. Frozen stocks of the cells were made within passage 6 and stored in liquid nitrogen. Cells were used for experiments below a passage number of 20.

Cell transfection: Cells were plated in a 96 well plate at a density of 10,000 cells per well. Transfections were carried out following 18-24 hours of incubation. The instructions provided with the Lipofectamine 3000 transfection reagent from Thermofisher Scientific were followed. Briefly, the transfection reagent was mixed with OptiMEM in one tube, and the ASO was mixed with the same in another tube. The contents of both the tubes were gently mixed together, and following an incubation, the transfection complex was layered onto the cells containing fresh medium. The cells were then incubated for the required time.

RNA isolation: Total RNA was extracted from cells treated with ASO for the required time periods using the Qiagen RNeasy minikit. Briefly, the cells were pelleted, and RNA was isolated according to the manufacturer's instructions. The genomic DNA elution column was used to free the RNA of any contaminating genomic DNA. RNA quantity and integrity were determined by nanodrop.

Reverse transcription: A total of 500 ug RNA was reverse transcribed to cDNA using the Promega M-MLV reverse transcriptase enzyme as per the kit instructions. The first strand synthesis of cDNA was performed using OligodT primers, which ensured that only mature polyadenylated RNA transcripts were reverse transcribed.

Intron retention analysis: cDNA from the various tissue/cell samples were analysed using the GoTaq® green kit provided by Promega. The cDNA was added to the reaction mastermix and pipetted into the qPCR plate. The primers for each exon-exon and exon-intron pair were then pipetted into the wells. Each reaction had a technical duplicate and a water control. Absence of genomic DNA contamination was ensured with the RT minus reactions.

Antisense oligonucleotide (ASO) screening using the Taqman® Fast Advanced Cells to Ct kit: Cells were seeded in 96 well plates at the required density. Within the following 18-24 hours, transfections were carried out and the cells were incubated up to the time of screening. The further experimental procedures were carried out according to the manufacturer's instructions. Briefly, the medium was aspirated, and the cells were washed with cold PBS. DNase-containing lysis solution was added to the cells and incubated for 5 min at room temperature. Stop solution was then added to the cells and the cells were incubated for 2 min at room temperature in order to halt the lysis. A total of 20% of lysate was used in the conversion to cDNA using the kit components for reverse transcription. The cDNA was then added to the master mix made using the components for qPCR provided in the kit at a concentration of 25%. Taqman® primers were used for the assay. Each reaction was duplexed with a housekeeping gene for in-well normalization of expression. The ASO treated cells were normalized to mock transfected cells.

Semiquantitative PCR: The cDNA was subjected to PCR using Taq polymerase from NEB with the suitable primers. The PCR products were separated on a 2% agarose gel and viewed using a gel documentation system.

### Example 2

### Intron Retention in Human SYNGAP1

### A. In Silico Analysis

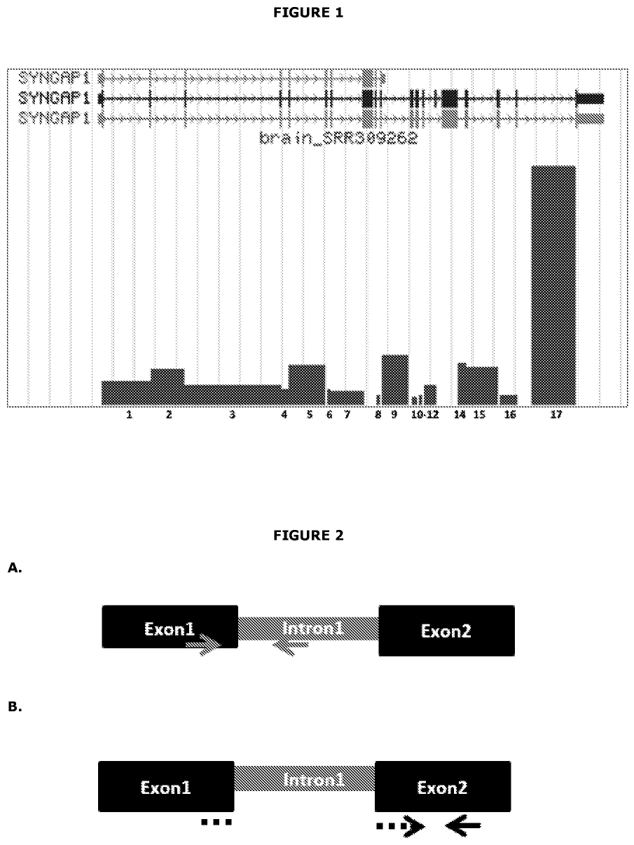

IRBase (Middleton et al., 2017, Genome Biol. 18: 51) is an RNA sequencing resource of over 2000 human samples, in which a specific intron retention event of a gene in a particular tissue can be assessed. Using this database, the events of intron retention of SYNGAP1 in brain tissue was analysed. As shown in FIG. 1, several introns of SYNGAP1 exhibit retention, with intron 17 showing the highest number of events (thin lines represent the introns and the thick lines/blocks correspond to the exons; the height of the bars is indicative of the number of recorded events of intron retention).

### B. In Vitro Validation

In vitro validation of the intron retention was carried out by analysing the levels of sequences corresponding to introns by quantitative PCR. To achieve this, primers were designed that would specifically detect the presence of retained introns relative to the exons flanking them. The use of cDNA that had been reverse transcribed from DNase-treated polyadenylated RNA ensured that pre-mRNA transcripts, and not genomic DNA, was detected by the primers.

Intron retention events in the mature SynGAP1 mRNA were analysed by real-time PCR. Two sets of primers were designed for each Exon-Intron pair across the SynGAP1 sequence (NM_006772; SEQ ID NO:1), consisting of 19 exons and 18 introns:

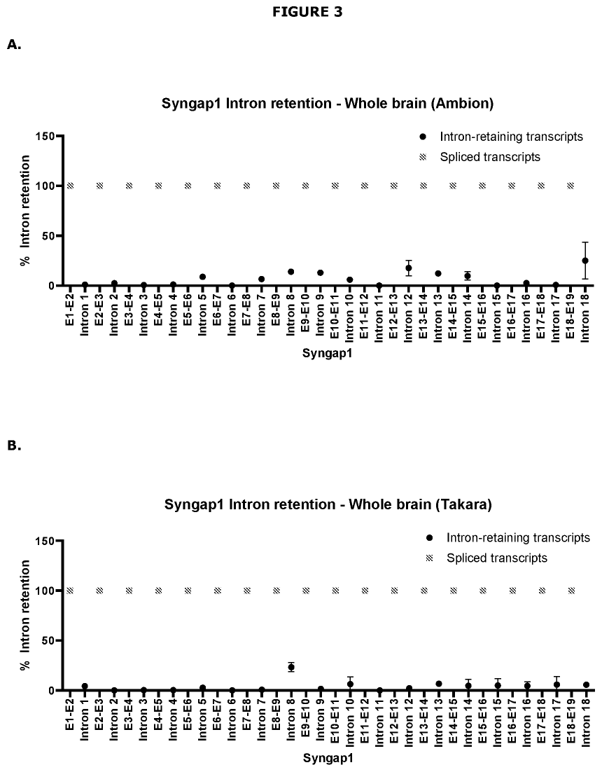

A: primers that are specific for intron-retaining transcripts: The forward primer was designed from the sequence of the preceding exon and the reverse primer from the sequence of the intron downstream to the exon (FIG. 2A).
B: primers specific to spliced transcripts: One of the primers was designed such that it spanned the junction of two nearby exons, while the other was designed from the sequence of the preceding or the succeeding exon accordingly (FIG. 2B).

The primers are set forth below in Table 1.

TABLE 1

| | | SEQ |
|---|---|---|
| Primer | Sequence | ID NO |
| SYN-E1-E2F | CTATGCCCCCTTCAGAGATGTAC | 11 |
| SYN-E1-E2R | TGGTTCCCCGAGATGATG | 12 |
| SYN-E1-I1F | CTCCACCTCCTCCTCCTTCT | 13 |
| SYN-E1-I1R | CCCACCACGTACCTCTGAAG | 14 |
| SYN-E2,3,4F | TCATGCTGGATGAGTATGAGATACA | 15 |
| SYN-E2,3,4R | CCCCTGGGACACTCTTCCT | 16 |
| SYN-E2-I2F | CTCTATGCACCGAACCCAAT | 17 |
| SYN-E2-I2R | GAGGACTCTCCCCATTCTCC | 18 |
| SYN-E3-I3F | TCGCAACAAACTGCTGAGAC | 19 |
| SYN-E3-I3R | ACTTTGTCTCCGCCTTCTCC | 20 |
| SYN-E4-E5F | CTCGCAAGGCTTCCTGAG | 21 |
| SYN-E4-E5R | CGGTCATGGTCAGCACTTC | 22 |
| SYN-E4-I4F | GCTCTAGGAGGAAGAGTGTC | 23 |
| SYN-E4-I4R | TGCCCTCTTTCTCAGACTCC | 24 |
| SYN-E5-E6F | GACCGTGCTCGGCTGAT | 25 |
| SYN-E5-E6R | TGCACTGGCTTGATAATGGA | 26 |
| SYN-E5-I5F | ACGGCTAAAAAGCTCCATCA | 27 |
| SYN-E5-I5R | CTCCCTCTGCATCTCACACA | 28 |
| SYN-E6-E7F | GGAGTGCTGTGTTGAGGTAACA | 29 |
| SYN-E6-E7R | TACTGCCCGCTCCAGATT | 30 |
| SYN-E6-I6F | GCCCAGGAGTTCTGTTTTGA | 31 |
| SYN-E6-I6R | CCTACCCTTTCCTCCAGTCC | 32 |
| SYN-E7-E8F | TAAAGCCCAACAAGGACAACA | 33 |
| SYN-E7-E8R | AGAAGACGGTGTCCCCAGAG | 34 |
| SYN-E7-I7F | GCCGAAAGAGACAAATGGAT | 35 |
| SYN-E7-I7R | CCCAAGCCTCTCCTCCTTTA | 36 |
| SYN-E8-E9F | GCCAAGCACTTCCTTTCAGAC | 37 |
| SYN-E8-E9R | CAATGGCATCCTTGAGGTATT | 38 |
| SYN-E8-I8F | CCATTATCGGATGCTGTGTG | 39 |
| SYN-E8-I8R | TGTCCTCACTAACCCCCAAG | 40 |
| SYN-E9-E10F | CATTGGAGAGTTCATCCGTG | 41 |
| SYN-E9-E10R | CAGTGGGAGTTGACCACCTT | 42 |
| SYN-E9-I9F | AACACGCTTGCCACTAAAGC | 43 |
| SYN-E9-I9R | TGTTGGAGGCTGGAGTCCTA | 44 |
| SYN-E10-E11F | TCAACTCCCACTGCGTGTTC | 45 |
| SYN-E10-E11R | TCGTGAGGTCTGCTCATCTG | 46 |
| SYN-E10-I10F | CTGCGAATGTGCTGTGAGTT | 47 |
| SYN-E10-I10R | AATTTGTCCCCATTCTGGTG | 48 |
| SYN-E11-E12F | CTTTTCCAAGTTTACCTCAAAGG | 49 |
| SYN-E11-E12R | CCAAGTCGATGTAACCCTCAA | 50 |
| SYN-E11-I11F | ATGAGCAGACCTCACGAACC | 51 |
| SYN-E11-I11R | TCATAGCCTCCCCATCTCAG | 52 |
| SYN-E12-E13F | AGCAAGGAATCCCTCCTGAA | 53 |
| SYN-E12-E13R | TGTAGCCCTGCATCTCAGC | 54 |
| SYN-E12-I12F | GACACGCTAACCAACAGCAG | 55 |
| SYN-E12-I12R | TCGAGAAACAAGGGGAGAAA | 56 |
| SYN-E13-E14F | CCTGAACGACATCAGCACAG | 57 |
| SYN-E13-E14R | TCGATGGAGCTGTTGAGGTC | 58 |
| SYN-E13-I13F | TGAGATGCACGGCTACATGA | 59 |
| SYN-E13-I13R | TCGTCTCCCCTGAGAACTTG | 60 |
| SYN-E14-E15F | TCAACAGCTCTATGGACATGG | 61 |
| SYN-E14-E15R | GCATCTTCTGCTCTGGCTCT | 62 |
| SYN-E14-I14F | TGCATCGACCTTCAGTCCTT | 63 |
| SYN-E14-I14R | GGGCACATATGGAGGAGATG | 64 |
| SYN-E15-E16F | ACCAAGCAGCATTCTCAGACA | 65 |
| SYN-E15-E16R | GGCTCTCATCCATCCATTTT | 66 |
| SYN-E15-I15F | CAGTCCCCAGAGCCAAGTTA | 67 |
| SYN-E15-I15R | TCTTCCCTCCCTGTTGTGAC | 68 |
| SYN-E16-E17F | GCTGGATAGGGTGAAGGAGTAC | 69 |
| SYN-E16-E17R | CTCCAGTCGGGCCTGATAC | 70 |
| SYN-E16-I16F | ATCGAGCGGGAAGAGTACAA | 71 |
| SYN-E16-I16R | AACTGACCCTGGAGGTTTCC | 72 |
| SYN-E17-E18F | ATAGAATCATTGGCAGGCTGA | 73 |
| SYN-E17-E18R | CAGAGCGTCGAGCATCCT | 74 |
| SYN-E17-I17F | GTCCCAGGAAGAACAAACCA | 75 |
| SYN-E17-I17R | CTCAGGCTCTCCCTCACAAC | 76 |
| SYN-E18-E19F | ACTCTCAGGAGAGGCAGCTTC | 77 |
| SYN-E18-E19R | TTCCTCGGTGATGTCCAATC | 78 |
| SYN-E18-I18F | CTGCCAGAACCCAAGAAGAG | 79 |
| SYN-E18-I18R | AGTGACAAAGGCACAGACGA | 80 |

### C. Intron Retention in Commercial Sources of Human Whole Brain RNA

Human brain RNA was used to check for intron retention in SynGAP1 mRNA. In order to include a wider spectrum of RNA samples, RNA from two commercial suppliers (Ambion and Takara) was obtained. As the primers span the intron, any genomic DNA isolated along with the RNA would be detected and could lead to an overestimation of intron retention. The RNA was therefore treated with DNase before reverse transcription. In addition, reactions without the reverse transcriptase enzyme were included to ensure the absence of genomic DNA. During reverse transcription of the RNA to cDNA, oligodT primers were used in order to favour the selection of transcripts that were mature (polyadenylated) and possessed retained introns. This prevented the reverse transcription of immature RNA transcripts whose introns were in the process of splicing.

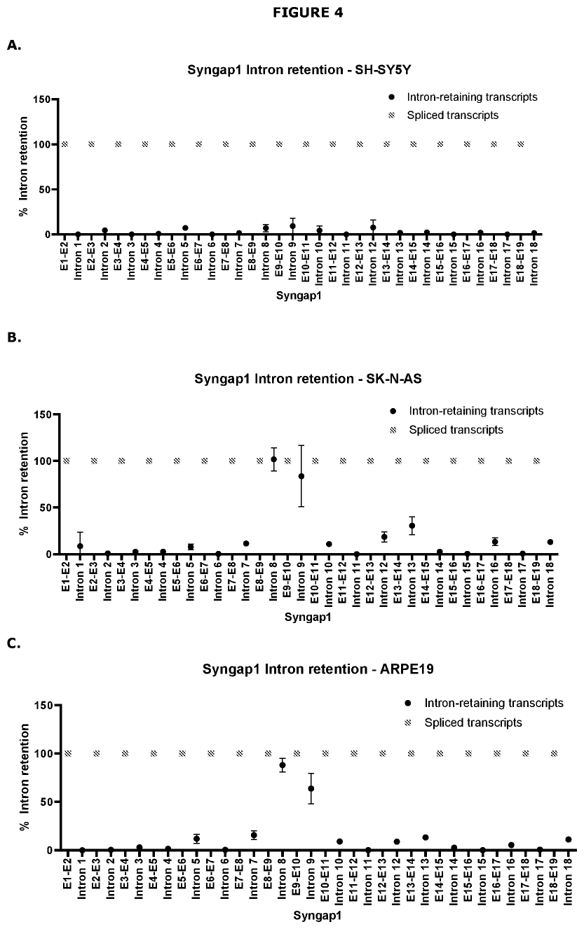

Using the strategy described above, a number of introns showing varying levels of retention in the first sample of human whole brain RNA (from Ambion) were detected, with the levels ranging from less than 10% to up to 40% of the expression of the exons. The introns showing the highest retention included introns 5, 8, 9, 12, 13, 14 and 18 (FIG. 3A). A somewhat different profile of intron retention was observed in the second source of human brain RNA (from Takara), where intron 8 showed the highest retention of 20% with respect to the exons (FIG. 3B).

In summary, several introns are retained to varying extents in SynGAP1 mature mRNA transcripts. Comparison of the retention profile between two different sources of RNA showed overlapping results with some introns showing retention in both samples. Intron 8 emerged as a common retained intron with similar levels of retention in the two samples.

### D. Intron Retention in the Cell Culture System

Intron retention in several cell lines, SH-SY5Y, SK-N-AS and ARPE19, was assessed as described above. SH-SY5Y and SK-N-AS are transformed neuronal-like cell lines that were derived from metastatic bone tumours. A human retinal pigment epithelial cell line, ARPE19, as also examined.

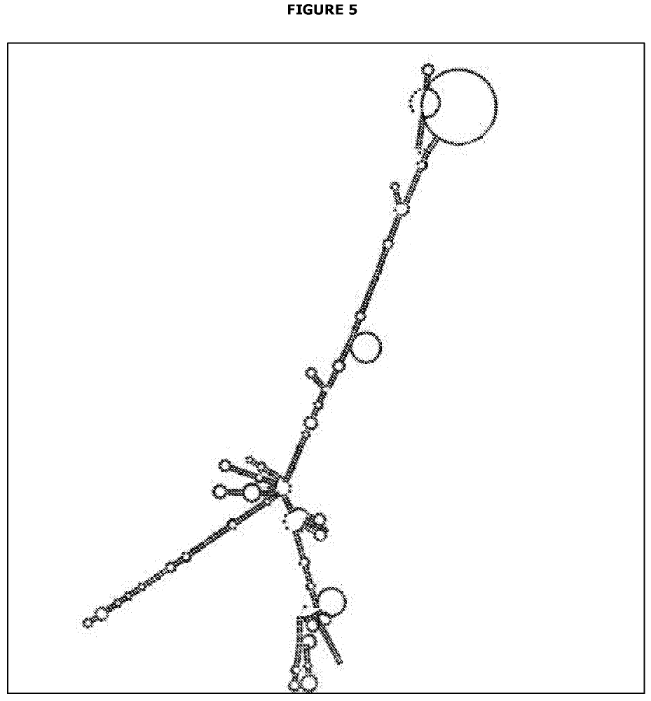

Similar to the intron retention profile observed in brain tissue, a few introns were retained up to 10% of the exon expression levels when SH-SY5Y cells were assessed (FIG. 4A). SK-N-AS cells showed high retention of introns 8 and 9, and although there was a high variation among biological replicates, the retention levels of these introns were >50% of the exon expression. The other introns in this cell line that showed retention were introns 7, 10, 12, 13, 16 and 18. In ARPE19 cells, introns 8 and 9, which showed highest retention in SH-SY5Y and SK-N-AS, as well as in one of the human brain samples sourced, also showed the highest retention. The other introns that showed retention were introns 5, 7, 13 and 18 (FIG. 4C).

In summary, among the introns retained in SynGAP1 mRNA, intron 8 (SEQ ID NO:6) and 9 (SEQ ID NO:7) show the highest retention levels among the different cell lines and sources tested.

### Example 3

### Identification of Antisense Oligonucleotides to Reduce Intro Retention

Targeting of an ASO to a particular sequence can sterically block the access of proteins, such as the spliceosome, to the nucleic acid molecule. Similarly, ASOs can be used to block sites such as splicing enhancer or silencer sequences, thereby altering the splicing propensity of a sequence. Blocking intronic splicing silencer (ISS) sites in the retained introns would in effect induce their splicing. Various tools and studies were therefore performed to identify ASOs that target the pool of SynGAP1 transcripts that bear retained introns and block ISS sites, so as to induce splicing and thereby increase the levels of spliced transcripts transported to the cytoplasm for translation into protein.

### A. Splicing Sequence Prediction Tools

Although ISS sequences serve as ideal antisense targets, they are often inconspicuous in long introns. To identify the part(s) of the intron sequence that would potentially increase splicing when targeted, available prediction tools (Human Splicing Finder, SpliceAid2, RBP Map, PESXs and RegRNA 2.0) were utilised for the in silico analysis of the intron sequences.

### B. QGRS Mapper

QGRS Mapper was used to predict the formation of G-quadruplexes in SynGAP1 pre-mRNA, which can be targeted by ASOs to induce splicing. G-quadruplexes are secondary structures that form between DNA/RNA when G quartets are linked by loop nucleotides. They have been reported to be involved in regulatory roles including translation and in the regulation of alternative splicing of pre-mRNA (Gomez et al, 2004, Nucleic Acids Res 32(1):371-9). Table 2 below shows the sequences identified using this process.

TABLE 2
QGRS sequences found (overlaps not included)

| | | | |
|---|---|---|---|
| 5 | 22 | GGATGATGGCTAGGGCTCAAGG | 20 |
| 71 | 17 | GGGGAGGCTGGGACAGG | 21 |
| 310 | 17 | GGATTGGGCAGGTAGGG | 21 |
| 396 | 20 | GGCAGGGGGTGAGGGCATGG | 21 |
| 429 | 25 | GGGGGCGGGGGGGGGGGTCCGGGGG | 82 |
| 497 | 28 | GGGGCTGCTATAAATAACTTCTTGGAGG | 2 |

### C. RNA Secondary Structure Prediction

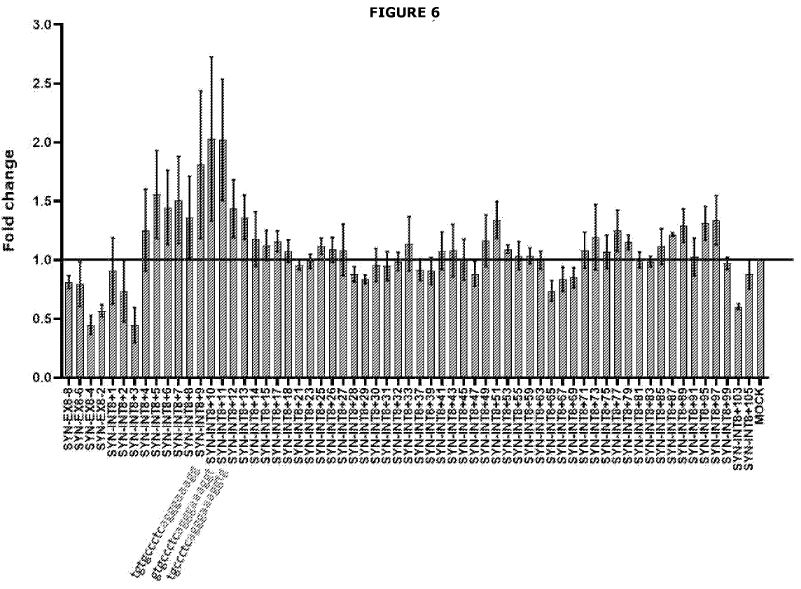

Poor splicing factor recruitment results in weakened splicing, and this has been reported to be affected by the secondary structure of the pre-mRNA (Buratti et al., 2004, Mol. Cell Biol. 24(24), 10505-10514). In-silico prediction of pre-mRNA secondary structure was performed using prediction tools such as MFold and RNA fold. FIG. 5 shows the secondary structure prediction of a sequence of intron 8 of SynGAP1. ASOs can be designed to target regions with high propensity for secondary structure.

d. Identifying Target Sequences

Based on published reports, the importance of the role of the splice repressors hnRNPA1 and hnRNP I on the splicing mechanism was inferred (Yimin Hua et al., 2008, Am. 3. Hum. Genet. 82, 834-848). The prediction tool SpliceAid2 (http://www.introni.it/splicing.html) was used to narrow down the regions of the intronic sequences near the 5′ and 3′ splice sites that might be bound by these splicing silencers. HnRNPA1 sites were predicted to be at positions 17-22 and 23-28 (CAGGGA and AAGGUG; i.e. spanning positions 17-28), and 57-62 (TAGTGA) from the 5′ splice site of the intron. There were no predicted hnRNP I binding sites in intron 8. Intron 9, which also showed significant retention, had a predicted HnRNPA1 binding site 104-108 bp from the 5′ splice site. The hnRNP I binding sites were at 21-29 bp (multiple putative overlapping sites at 21-26, 22-26, 22-28, 24-29 and 25-29 bp) and 190-195 bp from the 5′ splice site of intron 9.

### E. Design, Screening and Validation of ASOs Targeting Intron 8

1. Design and Screening of Intron 8 ASOs

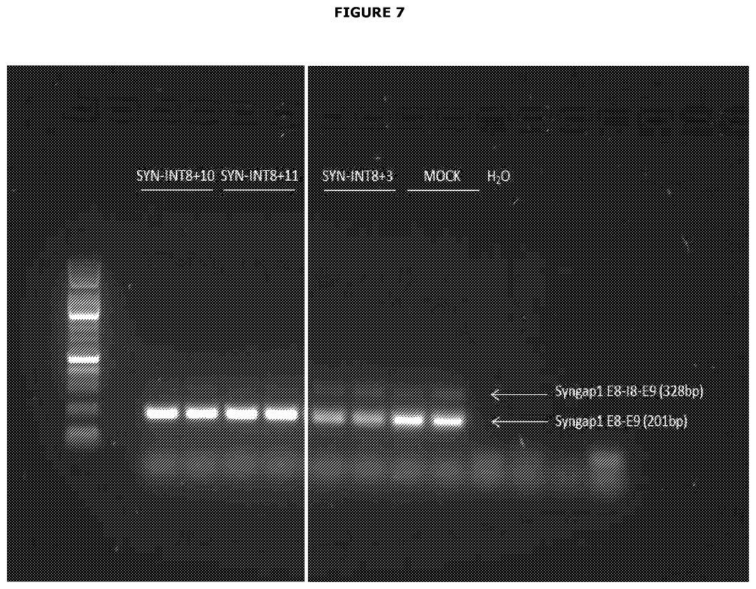

The ASOs were designed based on the predictions of the binding sites for the splicing repressors hnRNPA1 and hnRNP I, such that the ASOs would target those sites. The ASOs were 18 nucleotide-long, fully modified oligonucleotides with phosphorothioate (PS) backbone (to increase their stability) and 2′-O-methoxyethylribose (2′-MOE) sugar modifications (to increase binding affinity and reduce toxicity). A microwalk strategy was used to design the ASOs, starting from the 5′ end of intron 8 (see FIG. 6). ASOs with off-target binding sites were excluded. Table 3 sets forth the ASOs.

TABLE 3

| ASO Name | Sequence | SEQ ID NO. |
|---|---|---|
| SYN-EX8-8 | CAACACTCACCTTGGCCT | 192 |
| SYN-EX8-6 | CACAACACTCACCTTGGC | 193 |
| SYN-EX8-4 | GGCACAACACTCACCTTG | 194 |
| SYN-EX8-2 | AGGGCACAACACTCACCT | 195 |
| SYN-INT8+1 | TGAGGGCACAACACTCAC | 83 |
| SYN-INT8+2 | CTGAGGGCACAACACTCA | 84 |
| SYN-INT8+3 | CCTGAGGGCACAACACTC | 85 |
| SYN-INT8+4 | CCCTGAGGGCACAACACT | 86 |
| SYN-INT8+5 | TCCCTGAGGGCACAACAC | 87 |
| SYN-INT8+6 | TTCCCTGAGGGCACAACA | 88 |
| SYN-INT8+7 | TTTCCCTGAGGGCACAAC | 89 |
| SYN-INT8+8 | CTTTCCCTGAGGGCACAA | 90 |
| SYN-INT8+9 | CCTTTCCCTGAGGGCACA | 91 |
| SYN-INT8+10 | ACCTTTCCCTGAGGGCAC | 92 |
| SYN-INT8+11 | CACCTTTCCCTGAGGGCA | 93 |
| SYN-INT8+12 | TCACCTTTCCCTGAGGGC | 94 |
| SYN-INT8+13 | GTCACCTTTCCCTGAGGG | 95 |
| SYN-INT8+14 | AGTCACCTTTCCCTGAGG | 96 |
| SYN-INT8+15 | AAGTCACCTTTCCCTGAG | 97 |
| SYN-INT8+17 | CCAAGTCACCTTTCCCTG | 98 |
| SYN-INT8+18 | CCCAAGTCACCTTTCCCT | 99 |
| SYN-INT8+21 | ATTCCCAAGTCACCTTTC | 100 |
| SYN-INT8+23 | CCATTCCCAAGTCACCTT | 101 |
| SYN-INT8+25 | GCCCATTCCCAAGTCACC | 102 |
| SYN-INT8+26 | TGCCCATTCCCAAGTCAC | 103 |
| SYN-INT8+27 | GTGCCCATTCCCAAGTCA | 104 |
| SYN-INT8+28 | AGTGCCCATTCCCAAGTC | 105 |
| SYN-INT8+29 | AAGTGCCCATTCCCAAGT | 106 |
| SYN-INT8+30 | CAAGTGCCCATTCCCAAG | 107 |
| SYN-INT8+31 | GCAAGTGCCCATTCCCAA | 108 |
| SYN-INT8+32 | AGCAAGTGCCCATTCCCA | 109 |
| SYN-INT8+33 | AAGCAAGTGCCCATTCCC | 110 |
| SYN-INT8+37 | CCCCAAGCAAGTGCCCAT | 111 |
| SYN-INT8+39 | ACCCCCAAGCAAGTGCCC | 112 |
| SYN-INT8+41 | TAACCCCCAAGCAAGTGC | 113 |
| SYN-INT8+43 | ACTAACCCCCAAGCAAGT | 114 |
| SYN-INT8+45 | TCACTAACCCCCAAGCAA | 115 |
| SYN-INT8+47 | CCTCACTAACCCCCAAGC | 116 |
| SYN-INT8+49 | GTCCTCACTAACCCCCAA | 117 |
| SYN-INT8+51 | CTGTCCTCACTAACCCCC | 118 |
| SYN-INT8+53 | CCCTGTCCTCACTAACCC | 119 |
| SYN-INT8+55 | TGCCCTGTCCTCACTAAC | 120 |
| SYN-INT8+59 | AATTTGCCCTGTCCTCAC | 121 |
| SYN-INT8+63 | CGTGAATTTGCCCTGTCC | 122 |
| SYN-INT8+65 | CTCGTGAATTTGCCCTGT | 123 |
| SYN-INT8+67 | ATCTCGTGAATTTGCCCT | 124 |
| SYN-INT8+69 | CAATCTCGTGAATTTGCC | 125 |
| SYN-INT8+71 | CCCAATCTCGTGAATTTG | 126 |
| SYN-INT8+73 | AACCCAATCTCGTGAATT | 127 |
| SYN-INT8+75 | ACAACCCAATCTCGTGAA | 128 |
| SYN-INT8+77 | GCACAACCCAATCTCGTG | 129 |
| SYN-INT8+79 | CTGCACAACCCAATCTCG | 130 |
| SYN-INT8+81 | CTCTGCACAACCCAATCT | 131 |
| SYN-INT8+83 | GCCTCTGCACAACCCAAT | 132 |
| SYN-INT8+85 | CAGCCTCTGCACAACCCA | 133 |
| SYN-INT8+87 | GTCAGCCTCTGCACAACC | 134 |
| SYN-INT8+89 | GTGTCAGCCTCTGCACAA | 135 |
| SYN-INT8+91 | AAGTGTCAGCCTCTGCAC | 136 |
| SYN-INT8+95 | ATCCAAGTGTCAGCCTCT | 137 |
| SYN-INT8+97 | AAATCCAAGTGTCAGCCT | 138 |
| SYN-INT8+99 | GAAAATCCAAGTGTCAGC | 139 |
| SYN-INT8+103 | CCAGGAAAATCCAAGTGT | 140 |
| SYN-INT8+105 | GCCCAGGAAAATCCAAGT | 141 |
| SYN-INT8+107 | AGGCCCAGGAAAATCCAA | 142 |
| SYN-INT8+109 | TGAGGCCCAGGAAAATCC | 143 |

The ASOs were transfected into ARPE19 cells with lipofectamine 3000 at a concentration of 200 nM, and cells were lysed 24 hours later. Reverse transcription and qPCR analysis was performed as described in Example 1 using the Taqman® Fast Advance Cells to Ct kit (Thermofisher Scientific). A positive control (the ASO SYN-IVS15-36, which targets intron 15; WO2017/106377) was included in each screening round to ensure a functional screening system. The expression of SynGAP1 in cells following ASO treatment was compared and normalised with cells transfected with mock-transfected cells.

As shown in FIG. 6, among the ASOs tested, ASOs SYN-INT8+5 to SYN-INT8+11 caused an upregulation of 1.5 fold and above of SynGAP1 mRNA.

2. Validation of the Mechanism of Syngap1 Upregulation

In order to elucidate the mechanism of action of the ASOs on the upregulation of the SynGAP1 transcript, the levels of the two transcripts (those with retained introns and the mature transcripts) were determined using primers that could amplify both the transcripts. These primers bound the intron-flanking exons, i.e. exon 8 and 9.

TABLE 4

| Primer name | Sequence | SEQ ID NO: |
|---|---|---|
| SYN-E8-E9 | CCATTATCGGATGCTGTGTGC | 81 |
| (with I8) F | | |
| SYN-E8-E9 | GCTTTAGTGGCAAGCGTGTT | 82 |
| (with I8) R | | |

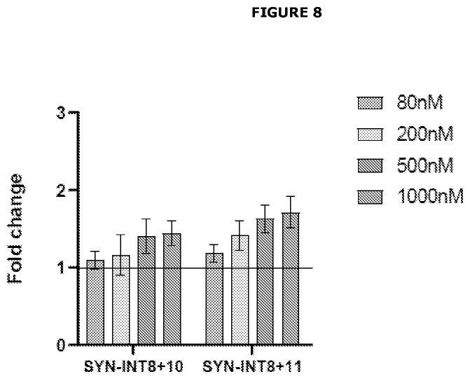

Following PCR of the RNA prepared from ASO-treated cells, the products were separated on a gel. As shown in FIG. 7, cells transfected with the ASOs that caused the highest upregulation of SynGAP1 mRNA (SYN-INT8+10 and SYN-INT8+11) had the least amount of intron-retaining transcripts, while the ASO that did not cause any upregulation (SYN-INT8+3) had a higher level of unspliced transcript. This suggested that ASOs that cause upregulation of SynGAP1 mRNA exert their effect through the splicing out of the retained intron 8.

3. Dose- and Time-Dependent Assessment of SYN-INT8+10 and SYN-INT8+11

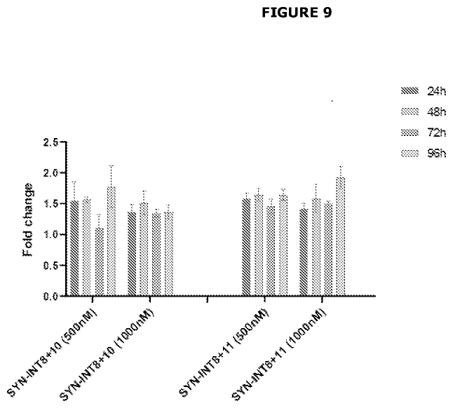

The ASOs that caused the highest upregulation of Syngap1, SYN-INT8+10 and SYN-INT8+11, were tested for a dose-dependent response in the upregulation of Syngap1. The cells were treated with concentrations varying from 80 nM to 1000 nM for 24 hours, and the expression of Syngap1 was analysed by qPCR. The response to the ASO was observed to increase with higher concentrations, but a saturating effect was observed at concentrations above 500 nM (FIG. 8).

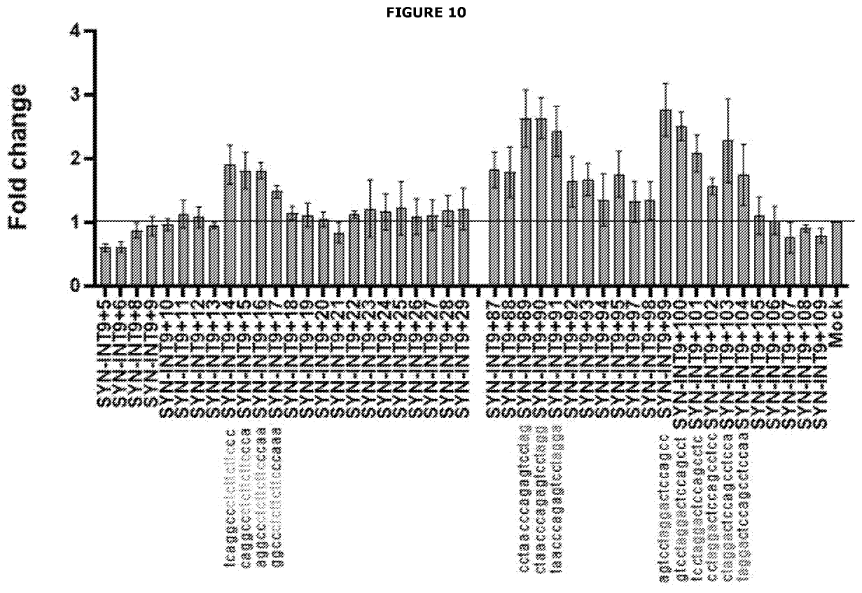

The time-dependent effect of the ASOs SYN-INT8+10 and SYN-INT8+11 on the upregulation of Syngap1 was also analysed. The ASOs were transfected in ARPE19 cells at a concentration of 500 nM & 1000 nM. Following 24-96 hours incubation, the expression of Syngap1 was analysed by qPCR. The upregulation caused by the ASOs lasted for up to 96 hours for both the tested ASOs. (FIG. 9).

### F. Design, Screening and Validation of ASOs Targeting Intron 9

1. Design and Screening of Intron 9 ASOs

The intron 9 ASOs were designed based on the predictions of the binding sites for the splicing repressors hnRNPA1 and hnRNPI, such that the ASOs would target those sites. The ASOs were 18 nucleotide-long, fully modified oligonucleotides with phosphorothioate (PS) backbone (to increase their stability) and 2′-O-methoxyethylribose (2′-MOE) sugar modifications (to increase binding affinity and reduce toxicity). A microwalk strategy was used to design the ASOs, starting from the 5′ end of intron 9. ASOs with off-target binding sites were excluded. Table 5 sets forth the ASOs.

TABLE 5

| ASO Name | Sequence | SEQ ID NO. |
|---|---|---|
| SYN-INT9+5 | AGGGCCTGAGTGTGGGCC | 144 |
| SYN-INT9+6 | GAGGGCCTGAGTGTGGGC | 145 |
| SYN-INT9+8 | AAGAGGGCCTGAGTGTGG | 146 |
| SYN-INT9+9 | GAAGAGGGCCTGAGTGTG | 147 |
| SYN-INT9+10 | AGAAGAGGGCCTGAGTGT | 148 |
| SYN-INT9+11 | AAGAAGAGGGCCTGAGTG | 149 |
| SYN-INT9+12 | GAAGAAGAGGGCCTGAGT | 150 |
| SYN-INT9+13 | GGAAGAAGAGGGCCTGAG | 151 |
| SYN-INT9+14 | GGGAAGAAGAGGGCCTGA | 152 |
| SYN-INT9+15 | TGGGAAGAAGAGGGCCTG | 153 |
| SYN-INT9+16 | TTGGGAAGAAGAGGGCCT | 154 |
| SYN-INT9+17 | TTTGGGAAGAAGAGGGCC | 155 |
| SYN-INT9+18 | GTTTGGGAAGAAGAGGGC | 156 |
| SYN-INT9+19 | GGTTTGGGAAGAAGAGGG | 157 |
| SYN-INT9+20 | AGGTTTGGGAAGAAGAGG | 158 |
| SYN-INT9+21 | CAGGTTTGGGAAGAAGAG | 159 |
| SYN-INT9+22 | GCAGGTTTGGGAAGAAGA | 160 |
| SYN-INT9+23 | GGCAGGTTTGGGAAGAAG | 161 |
| SYN-INT9+24 | TGGCAGGTTTGGGAAGAA | 162 |
| SYN-INT9+25 | CTGGCAGGTTTGGGAAGA | 163 |
| SYN-INT9+26 | TCTGGCAGGTTTGGGAAG | 164 |
| SYN-INT9+27 | ATCTGGCAGGTTTGGGAA | 165 |
| SYN-INT9+28 | CATCTGGCAGGTTTGGGA | 166 |
| SYN-INT9+29 | ACATCTGGCAGGTTTGGG | 167 |
| SYN-INT9+87 | AGGACTCTGGGTTAGGAA | 168 |
| SYN-INT9+88 | TAGGACTCTGGGTTAGGA | 169 |
| SYN-INT9+89 | CTAGGACTCTGGGTTAGG | 170 |
| SYN-INT9+90 | CCTAGGACTCTGGGTTAG | 171 |
| SYN-INT9+91 | TCCTAGGACTCTGGGTTA | 172 |
| SYN-INT9+92 | GTCCTAGGACTCTGGGTT | 173 |
| SYN-INT9+93 | AGTCCTAGGACTCTGGGT | 174 |
| SYN-INT9+94 | GAGTCCTAGGACTCTGGG | 175 |
| SYN-INT9+95 | GGAGTCCTAGGACTCTGG | 176 |
| SYN-INT9+97 | CTGGAGTCCTAGGACTCT | 177 |
| SYN-INT9+98 | GCTGGAGTCCTAGGACTC | 178 |
| SYN-INT9+99 | GGCTGGAGTCCTAGGACT | 179 |
| SYN-INT9+100 | AGGCTGGAGTCCTAGGAC | 180 |
| SYN-INT9+101 | GAGGCTGGAGTCCTAGGA | 181 |
| SYN-INT9+102 | GGAGGCTGGAGTCCTAGG | 182 |
| SYN-INT9+103 | TGGAGGCTGGAGTCCTAG | 183 |
| SYN-INT9+104 | TTGGAGGCTGGAGTCCTA | 184 |
| SYN-INT9+105 | GTTGGAGGCTGGAGTCCT | 185 |
| SYN-INT9+106 | TGTTGGAGGCTGGAGTCC | 186 |
| SYN-INT9+107 | GTGTTGGAGGCTGGAGTC | 187 |
| SYN-INT9+108 | GGTGTTGGAGGCTGGAGT | 188 |
| SYN-INT9+109 | AGGTGTTGGAGGCTGGAG | 189 |

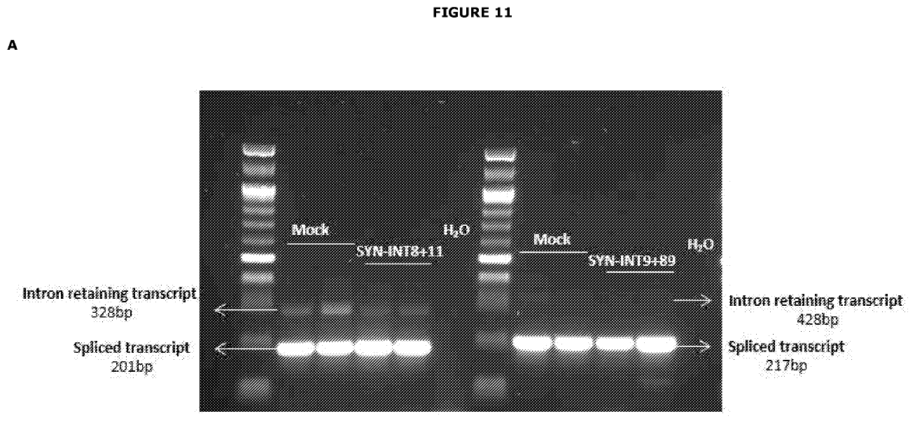

As shown in FIG. 10, among the ASOs tested, several ASOs caused an upregulation of higher than 1.5 fold upregulation in the expression of Syngap1 mRNA as compared to mock transfected cells. SYN-INT9+89, SYN-INT9+90, SYN-INT9+91 and SYN-INT9+99 caused upregulation of 2.5 fold and above of SynGAP1 mRNA.

2. Validation of the Mechanism of Syngap1 Upregulation

In order to elucidate the mechanism of action of the ASOs on the upregulation of the SynGAP1 transcript, the levels of the two transcripts (those with retained introns and the mature transcripts) were determined using primers that could amplify both the transcripts. These primers (see Table 6) bound the intron-flanking exons, i.e. exon 9 and 10.

TABLE 6

| Primer name | Sequence | SEQ ID NO: |
|---|---|---|
| hSYN-E9-I9- | AACACGCTTGCCACTAAAGC | 190 |
| E10F | | |
| hSYN-E9-I9- | AGTGGGAGTTGACCACCTTG | 191 |
| E10R | | |

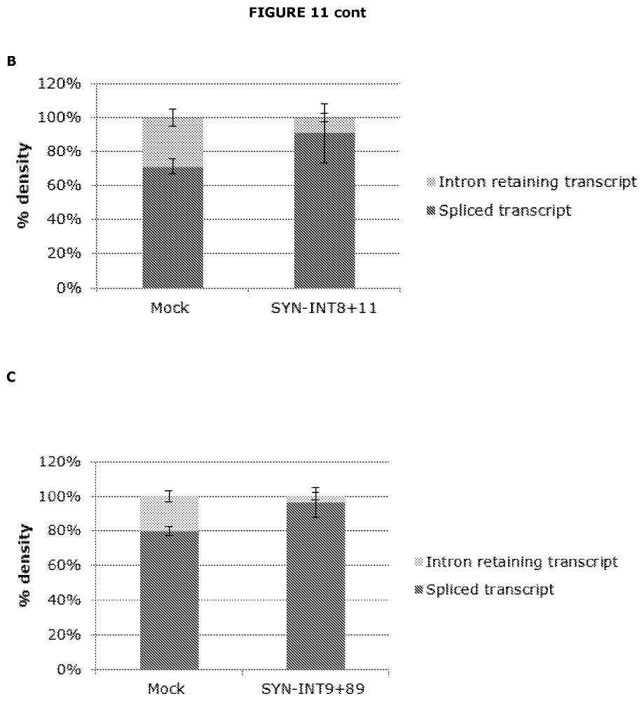

Following PCR of the RNA prepared from ASO-treated cells, the products were separated on a gel. As shown in FIG. 11, cells transfected with the intron 9 ASO that caused the highest upregulation of SynGAP1 mRNA (SYN-INT9+89) had less amount of intron-retaining transcripts as compared to the mock transfected cells. This suggested that this and other intron 9 ASOs that cause upregulation of SynGAP1 mRNA exert their effect through the splicing out of the retained intron 9. The effect of the intron 8-targeting ASO, SYN-INT8+11, was also assessed for comparison.

### Other Sequences Referred to in the Disclosure

SEQ ID NO: 1; SynGAP1 transcript 1 (NM_006772):
CTCTCTCTCGGCTGCCGCTGCTGCCGTTGGCTCTTATTCTCCTCCTCCTCCTCCTCTCTCCTCCTCTCTG
CTTCTCTCTGCTCCTCTCTCCTCCTCTCTCCTCCTCCTCCTCCTCCACCTCCTCCTCCTTCTCCCCCTCTT
TCTCCCCCTCTTTCTCTCTTCTTTCTCCCCCGTCCCCCCGCCCCCTCCCCCCAGGCCTGATGAGCAGGTC
TCGAGCCTCCATCCATCGGGGGAGCATCCCCGCGATGTCCTATGCCCCCTTCAGAGATGTACGGGGAC
CCTCTATGCACCGAACCCAATACGTTCATTCCCCGTATGATCGTCCTGGTTGGAACCCTCGGTTCTGCA
TCATCTCGGGGAACCAGCTGCTCATGCTGGATGAGGATGAGATACACCCCCTACTGATCCGGGACCGG
AGGAGCGAGTCCAGTCGCAACAAACTGCTGAGACGCACAGTCTCCGTGCCGGTGGAGGGGCGGCCCC
ACGGCGAGCATGAATACCACTTGGGTCGCTCGAGGAGGAAGAGTGTCCCAGGGGGGAAGCAGTACAG
CATGGAGGGTGCCCCTGCTGCGCCCTTCCGGCCCTCGCAAGGCTTCCTGAGCCGACGGCTAAAAAGCT
CCATCAAACGAACGAAGTCACAACCCAAACTTGACCGGACCAGCAGCTTTCGCCAGATCCTGCCTCGCT
TCCGAAGTGCTGACCATGACCGGGCCCGGCTGATGCAAAGCTTTAAGGAGTCACACTCTCATGAGTCC
TTGCTGAGTCCTAGCAGTGCAGCTGAGGCATTGGAGCTCAACTTGGATGAAGATTCCATTATCAAGCCA
GTGCACAGCTCCATCCTGGGCCAGGAGTTCTGTTTTGAGGTAACAACTTCATCAGGAACAAAATGCTTT
GCCTGTCGGTCTGCGGCCGAAAGAGACAAATGGATTGAGAATCTGCAGCGGGCAGTAAAGCCCAACAA
GGACAACAGCCGCCGGGTAGACAATGTGCTAAAGCTGTGGATCATAGAGGCCCGGGAGCTGCCCCCC
AAGAAGCGGTACTACTGTGAGCTCTGCCTGGATGACATGCTGTATGCACGCACCACCTCCAAGCCCCG
CTCTGCCTCTGGGGACACCGTCTTCTGGGGCGAGCACTTCGAGTTTAACAACCTGCCGGCTGTCCGTG
CCCTGCGGCTGCATCTGTACCGTGACTCAGACAAAAAGCGCAAGAAGGACAAGGCAGGCTATGTCGGC
CTGGTGACTGTGCCAGTGGCCACCCTGGCTGGGCGCCACTTCACAGAGCAGTGGTACCCTGTAACCCT
GCCAACAGGCAGTGGGGGATCTGGGGGCATGGGTTCGGGAGGGGGAGGGGGCTCGGGGGGTGGCT
CAGGGGGCAAGGGCAAAGGAGGTTGCCCGGCTGTGCGGCTGAAAGCACGTTACCAGACAATGAGCAT
CTTGCCCATGGAGCTATATAAAGAGTTTGCAGAGTATGTCACCAACCATTATCGGATGCTGTGTGCAGT
CTTGGAGCCCGCCCTGAATGTCAAAGGCAAGGAGGAGGTTGCCAGTGCACTAGTTCACATCCTGCAGA
GTACAGGCAAGGCCAAGGACTTCCTTTCAGACATGGCCATGTCTGAGGTAGACCGGTTCATGGAACGG
GAGCACCTCATATTCCGCGAGAACACGCTTGCCACTAAAGCCATAGAAGAGTATATGAGACTGATTGGT
CAGAAATACCTCAAGGATGCCATTGGAGAATTCATCCGTGCTCTGTATGAATCTGAGGAAAACTGCGAG
GTAGACCCTATCAAGTGCACAGCATCCAGTTTGGCAGAGCACCAGGCCAACCTGCGAATGTGCTGTGA
GTTGGCCCTGTGCAAGGTGGTCAACTCCCACTGCGTGTTCCCGAGGGAGCTGAAGGAGGTGTTTGCTT
CGTGGCGGCTGCGCTGCGCAGAGCGAGGCCGGGAGGACATCGCAGACAGGCTTATCAGCGCCTCACT
CTTCCTGCGCTTCCTCTGCCCAGCGATTATGTCGCCCAGTCTCTTTGGGCTTATGCAGGAGTACCCAGA
TGAGCAGACCTCACGAACCCTCACCCTCATTGCCAAGGTCATCCAGAACCTGGCCAACTTTTCCAAGTT
TACCTCAAAGGAGGACTTTCTGGGCTTCATGAATGAGTTTCTGGAGCTGGAATGGGGTTCCATGCAGCA
GTTTTTGTATGAGATCTCCAATCTGGACACGCTAACCAACAGCAGTAGCTTTGAGGGTTACATCGACTT
GGGCCGAGAGCTCTCCACACTGCATGCCCTACTCTGGGAGGTGCTGCCCCAGCTCAGCAAGGAAGCCC
TCCTGAAGCTGGGTCCACTGCCCCGGCTCCTCAACGACATCAGCACAGCTCTGAGGAACCCCAACATC
CAAAGGCAGCCAAGCCGCCAGAGTGAGCGGCCCCGGCCTCAGCCTGTGGTACTGCGGGGGCCATCGG
CTGAGATGCAGGGCTACATGATGCGGGACCTCAACAGCTCCATCGACCTTCAGTCCTTCATGGCTCGA
GGCCTCAACAGCTCTATGGACATGGCTCGCCTCCCCTCCCCAACCAAGGAAAAGCCACCCCCACCACC
GCCTGGTGGTGGTAAAGACCTGTTCTATGTAAGCCGTCCACCCCTGGCCCGTTCCTCACCAGCATACTG
CACGAGCAGCTCGGACATCACAGAGCCAGAGCAGAAGATGCTGAGTGTCAACAAGAGTGTGTCCATGC
TGGACTTACAGGGTGATGGGCCTGGTGGCCGCCTCAACAGCAGCAGTGTTTCGAACCTGGCGGCCGTA
GGGGACCTGCTGCACTCAAGCCAGGCCTCGCTGACAGCAGCCTTGGGGCTACGGCCTGCGCCTGCCG
GACGCCTCTCCCAGGGGAGTGGCTCATCCATCACGGCGGCTGGCATGCGCCTCAGCCAGATGGGTGT
CACCACAGACGGTGTCCCTGCCCAGCAACTGCGAATCCCCCTCTCCTTCCAGAACCCTCTCTTCCACAT
GGCTGCTGATGGGCCAGGTCCCCCAGGCGGCCATGGAGGGGGCGGTGGCCATGGCCCACCTTCCTCC
CATCACCACCACCACCACCATCACCACCACCGAGGTGGAGAGCCCCCTGGGGACACCTTTGCCCCATT
CCATGGCTATAGCAAGAGTGAGGACCTCTCTTCCGGGGTCCCCAAGCCCCCTGCTGCCTCCATCCTTCA
TAGCCACAGCTACAGTGATGAGTTTGGACCCTCTGGCACTGACTTCACCCGTCGGCAGCTTTCACTCCA
GGACAACCTGCAGCACATGCTGTCCCCTCCCCAGATCACCATTGGTCCCCAGAGGCCAGCCCCCTCAG
GGCCTGGAGGTGGGAGCGGTGGGGGCAGCGGTGGGGGTGGCGGGGGCCAGCCGCCTCCATTGCAG
AGGGGCAAGTCTCAGCAGTTGACAGTCAGCGCAGCCCAGAAACCCCGGCCATCCAGCGGGAATCTATT
GCAGTCCCCAGAGCCAAGTTATGGCCCCGCCCGTCCACGGCAACAGAGCCTCAGCAAGGAGGGCAGC
ATTGGGGGCAGCGGGGGCAGCGGTGGCGGAGGGGGTGGGGGGCTGAAGCCCTCCATCACCAAGCAG
CATTCTCAGACACCATCCACATTGAACCCCACAATGCCAGCCTCTGAGCGGACAGTGGCCTGGGTCTCC
AACATGCCTCACCTGTCGGCTGACATCGAGAGTGCCCACATCGAGCGGGAAGAGTACAAGCTCAAGGA
GTACTCAAAATCGATGGATGAGAGCCGGCTGGATAGGGTGAAGGAGTACGAGGAGGAGATTCACTCA
CTGAAAGAGCGGCTGCACATGTCCAACCGGAAGCTGGAAGAGTATGAGCGGAGGCTGCTGTCCCAGG
AAGAACAAACCAGCAAAATCCTGATGCAGTATCAGGCCCGACTGGAGCAGAGTGAGAAGAGGCTAAGG
CAGCAGCAGGCAGAGAAGGATTCCCAGATCAAGAGCATCATTGGCAGGCTGATGCTGGTGGAGGAGG
AGCTGCGCCGGGACCACCCCGCCATGGCTGAGCCGCTGCCAGAACCCAAGAAGAGGCTGCTCGACGC
TCAGGAGAGGCAGCTTCCCCCCTTGGGTCCAACAAACCCGCGTGTGACGCTGGCCCCACCGTGGAATG
GCCTGGCCCCCCCAGCCCCACCACCCCCACCCCGGCTGCAGATTACGGAGAACGGCGAGTTCCGAAAC
ACCGCAGACCACTAGCCCACCCAGCATCAGAGACCTTCTCTTCCTTTCCTGTGCACCCCACCCTGTAAC
AGCACCAACCACCAGGATTGGACATCACCGAGGAACAGCGGGATTGCCTCCCCGAATGCCTCCCTGGG
AGGCACACTGATTGCCCACCCCCACCACTGCACCATTTCCAGGAGGGAGAGTGGGGACCCTCAGCCGC
CCCCTTTTCCTTCCCATTGGGGTGCTGCCCTCTCTTTGACCCCCAGGGACCCTTGCCCCAGGACACCGC
CTACCCCGTACAGACCCCTTCACTCCGGGGTGCTATCCCCATCCTCTGCCTCATCGTTCCCCTGAGCAC
TGGGGGACAGACCCTCACCCCCACCCTGGGGGTGTGGCACCTCCAAACTTTCAACTTCAGGGTGATTT
TTTTAGCAGTAACCAGAGCTGACAATCTAACTCCCCTCCACCGCCCCATTTTGGCCTCCCCTGCCCCCCT
TGTTATGGGGAGGGGACCCCGGGTGAGGGGGCCCTATTACCCCTTGATTTCTCAGGAGCGTCTGGGG
GGGCTCAGCACGCACAAACTCCTTCTCCTTCTACCACTCTTAAATTTACTCCCTCCCCACCCAGAACCCA
GATGGGGTGGAGGGGGCCACCGGGGCAGGGAGGGGGCGGCAAGGGGGGAATGGGAGTTGTCTCCC
CTTCTCCCCACACCTGATCTGCTCTCGGCTGGTCCCAGAGCGGGGTGAGGGGGCTTATGCCCCCCCCT
CCCCCAGTGTGTTGGGTGGGGTGGAATTGAGGTTAGGGTGAGGGGTCAGGGTTTAGGAGGGTGTGTA
TGTTGGGAGGACAGGCTAGTTGATCTGTCCTACTCTGACACACAGTCCCCTCTGCCCCTTCCTTCTCTCT
TCTTGGTCTCTACTCCCAGGGGGAGGGGGGAACTTACTCTAGGAAAAGCCATGTCTCTCTCCCCCAGG
GTGGGGGGACCTGTGTTGGAGGAGGGGTGTTGGGGGGCCCCCTTCCATGACTCTGTCCCCTGGGGGA
GGTAGGACAGGGCTGGGCTTCCCTCTCATCCTCCCCCTCCCAATCTCCTTCCACCTCCCTCCCTCCCGC
CAGCTCCACGATTTTTCGGTGTTTCTCTGTACATAGTTTTCTGGCGGGATAGGGGAGGTAGGATGGATG
GGGTTTGGGGTGGGTAGGCCATGGGAGGGGAGAAGCCCCTCCTTGGCACCCCCTCTTCCCTGACTGC
TGTCCCCTACCCAGCCTTGCCCCCTTCATCCTTTTGCGTTTGGTATTGAGACTCTCCTAGACTCTACTCC
TCTTTCTTTTGTATGGACAGTTCCCCTTCAGTCCCATCCCCCTACACATACACCCAGCCGGGGCCAAATT
TATACTTATATAAAAGTTGTAAATATGTGAAATTTTATCCCTGTGCCCTTTCCCCACCTCAGGCCCTACCC
CTGGACCCTCCCCAACCTTCCTTCTCTCTTCTTTGGCTGTTGTAATTATCTGGGGTTTGTACTGTACATAT
CCGGGGT6T6T6T6T6TGGGCTGGGGGCAACCCTTCTGTACAGAGCTTCCTGGCCCCCTCCCCCCCCG
CCCCTCTGCTTCCCTCCCCACCCACCACCTCAAGGGTAGGGAGTTGCTCTTCCTACCTGTTTTATTTTGT
TTTCTCGTTCTCCCTCCCCACCCCACTCCCAGCCTTATCTATCCCCCCTCACTGTCCCCTTTTCTCCACTC
CCAGCCCCATTTCCTTTTTTTCTGGAGTGTGTGGTGAAACAGAAAAAAACATGTTTAATAAACGGAGATT
GTTCTTTTA
SEQ ID NO: 2; SynGAP1 transcript 2 (NM_001130066.2):
CTCTCTCTCGGCTGCCGCTGCTGCCGTTGGCTCTTATTCTCCTCCTCCTCCTCCTCTCTCCTCCTCTCTG
CTTCTCTCTGCTCCTCTCTCCTCCTCTCTCCTCCTCCTCCTCCTCCACCTCCTCCTCCTTCTCCCCCTCTT
TCTCCCCCTCTTTCTCTCTTCTTTCTCCCCCGTCCCCCCGCCCCCTCCCCCCAGGCCTGATGAGCAGGTC
TCGAGCCTCCATCCATCGGGGGAGCATCCCCGCGATGTCCTATGCCCCCTTCAGAGATGTACGGGGAC
CCTCTATGCACCGAACCCAATACGTTCATTCCCCGTATGATCGTCCTGGTTGGAACCCTCGGTTCTGCA
TCATCTCGGGGAACCAGCTGCTCATGCTGGATGAGGATGAGATACACCCCCTACTGATCCGGGACCGG
AGGAGCGAGTCCAGTCGCAACAAACTGCTGAGACGCACAGTCTCCGTGCCGGTGGAGGGGCGGCCCC
ACGGCGAGCATGAATACCACTTGGGTCGCTCGAGGAGGAAGAGTGTCCCAGGGGGGAAGCAGTACAG
CATGGAGGGTGCCCCTGCTGCGCCCTTCCGGCCCTCGCAAGGCTTCCTGAGCCGACGGCTAAAAAGCT
CCATCAAACGAACGAAGTCACAACCCAAACTTGACCGGACCAGCAGCTTTCGCCAGATCCTGCCTCGCT
TCCGAAGTGCTGACCATGACCGGGCCCGGCTGATGCAAAGCTTTAAGGAGTCACACTCTCATGAGTCC
TTGCTGAGTCCTAGCAGTGCAGCTGAGGCATTGGAGCTCAACTTGGATGAAGATTCCATTATCAAGCCA
GTGCACAGCTCCATCCTGGGCCAGGAGTTCTGTTTTGAGGTAACAACTTCATCAGGAACAAAATGCTTT
GCCTGTCGGTCTGCGGCCGAAAGAGACAAATGGATTGAGAATCTGCAGCGGGCAGTAAAGCCCAACAA
GGACAACAGCCGCCGGGTAGACAATGTGCTAAAGCTGTGGATCATAGAGGCCCGGGAGCTGCCCCCC
AAGAAGCGGTACTACTGTGAGCTCTGCCTGGATGACATGCTGTATGCACGCACCACCTCCAAGCCCCG
CTCTGCCTCTGGGGACACCGTCTTCTGGGGCGAGCACTTCGAGTTTAACAACCTGCCGGCTGTCCGTG
CCCTGCGGCTGCATCTGTACCGTGACTCAGACAAAAAGCGCAAGAAGGACAAGGCAGGCTATGTCGGC
CTGGTGACTGTGCCAGTGGCCACCCTGGCTGGGCGCCACTTCACAGAGCAGTGGTACCCTGTAACCCT
GCCAACAGGCAGTGGGGGATCTGGGGGCATGGGTTCGGGAGGGGGAGGGGGCTCGGGGGGTGGCT
CAGGGGGCAAGGGCAAAGGAGGTTGCCCGGCTGTGCGGCTGAAAGCACGTTACCAGACAATGAGCAT
CTTGCCCATGGAGCTATATAAAGAGTTTGCAGAGTATGTCACCAACCATTATCGGATGCTGTGTGCAGT
CTTGGAGCCCGCCCTGAATGTCAAAGGCAAGGAGGAGGTTGCCAGTGCACTAGTTCACATCCTGCAGA
GTACAGGCAAGGCCAAGGACTTCCTTTCAGACATGGCCATGTCTGAGGTAGACCGGTTCATGGAACGG
GAGCACCTCATATTCCGCGAGAACACGCTTGCCACTAAAGCCATAGAAGAGTATATGAGACTGATTGGT
CAGAAATACCTCAAGGATGCCATTGGAGAATTCATCCGTGCTCTGTATGAATCTGAGGAAAACTGCGAG
GTAGACCCTATCAAGTGCACAGCATCCAGTTTGGCAGAGCACCAGGCCAACCTGCGAATGTGCTGTGA
GTTGGCCCTGTGCAAGGTGGTCAACTCCCACTGCGTGTTCCCGAGGGAGCTGAAGGAGGTGTTTGCTT
CGTGGCGGCTGCGCTGCGCAGAGCGAGGCCGGGAGGACATCGCAGACAGGCTTATCAGCGCCTCACT
CTTCCTGCGCTTCCTCTGCCCAGCGATTATGTCGCCCAGTCTCTTTGGGCTTATGCAGGAGTACCCAGA
TGAGCAGACCTCACGAACCCTCACCCTCATTGCCAAGGTCATCCAGAACCTGGCCAACTTTTCCAAGTT
TACCTCAAAGGAGGACTTTCTGGGCTTCATGAATGAGTTTCTGGAGCTGGAATGGGGTTCCATGCAGCA
GTTTTTGTATGAGATCTCCAATCTGGACACGCTAACCAACAGCAGTAGCTTTGAGGGTTACATCGACTT
GGGCCGAGAGCTCTCCACACTGCATGCCCTACTCTGGGAGGTGCTGCCCCAGCTCAGCAAGGAAGCCC
TCCTGAAGCTGGGTCCACTGCCCCGGCTCCTCAACGACATCAGCACAGCTCTGAGGAACCCCAACATC
CAAAGGCAGCCAAGCCGCCAGAGTGAGCGGCCCCGGCCTCAGCCTGTGGTACTGCGGGGGCCATCGG
CTGAGATGCAGGGCTACATGATGCGGGACCTCAACAGCTCTATGGACATGGCTCGCCTCCCCTCCCCA
ACCAAGGAAAAGCCACCCCCACCACCGCCTGGTGGTGGTAAAGACCTGTTCTATGTAAGCCGTCCACC
CCTGGCCCGTTCCTCACCAGCATACTGCACGAGCAGCTCGGACATCACAGAGCCAGAGCAGAAGATGC
TGAGTGTCAACAAGAGTGTGTCCATGCTGGACTTACAGGGTGATGGGCCTGGTGGCCGCCTCAACAGC
AGCAGTGTTTCGAACCTGGCGGCCGTAGGGGACCTGCTGCACTCAAGCCAGGCCTCGCTGACAGCAG
CCTTGGGGCTACGGCCTGCGCCTGCCGGACGCCTCTCCCAGGGGAGTGGCTCATCCATCACGGCGGC
TGGCATGCGCCTCAGCCAGATGGGTGTCACCACAGACGGTGTCCCTGCCCAGCAACTGCGAATCCCCC
TCTCCTTCCAGAACCCTCTCTTCCACATGGCTGCTGATGGGCCAGGTCCCCCAGGCGGCCATGGAGGG
GGCGGTGGCCATGGCCCACCTTCCTCCCATCACCACCACCACCACCATCACCACCACCGAGGTGGAGA
GCCCCCTGGGGACACCTTTGCCCCATTCCATGGCTATAGCAAGAGTGAGGACCTCTCTTCCGGGGTCC
CCAAGCCCCCTGCTGCCTCCATCCTTCATAGCCACAGCTACAGTGATGAGTTTGGACCCTCTGGCACTG
ACTTCACCCGTCGGCAGCTTTCACTCCAGGACAACCTGCAGCACATGCTGTCCCCTCCCCAGATCACCA
TTGGTCCCCAGAGGCCAGCCCCCTCAGGGCCTGGAGGTGGGAGCGGTGGGGGCAGCGGTGGGGGTG
GCGGGGGCCAGCCGCCTCCATTGCAGAGGGGCAAGTCTCAGCAGTTGACAGTCAGCGCAGCCCAGAA
ACCCCGGCCATCCAGCGGGAATCTATTGCAGTCCCCAGAGCCAAGTTATGGCCCCGCCCGTCCACGGC
AACAGAGCCTCAGCAAGGAGGGCAGCATTGGGGGCAGCGGGGGCAGCGGTGGCGGAGGGGGTGGG
GGGCTGAAGCCCTCCATCACCAAGCAGCATTCTCAGACACCATCCACATTGAACCCCACAATGCCAGCC
TCTGAGCGGACAGTGGCCTGGGTCTCCAACATGCCTCACCTGTCGGCTGACATCGAGAGTGCCCACAT
CGAGCGGGAAGAGTACAAGCTCAAGGAGTACTCAAAATCGATGGATGAGAGCCGGCTGGATAGGGAG
TACGAGGAGGAGATTCACTCACTGAAAGAGCGGCTGCACATGTCCAACCGGAAGCTGGAAGAGTATGA
GCGGAGGCTGCTGTCCCAGGAAGAACAAACCAGCAAAATCCTGATGCAGTATCAGGCCCGACTGGAGC
AGAGTGAGAAGAGGCTAAGGCAGCAGCAGGCAGAGAAGGATTCCCAGATCAAGAGCATCATTGGCAG
GCTGATGCTGGTGGAGGAGGAGCTGCGCCGGGACCACCCCGCCATGGCTGAGCCGCTGCCAGAACCC
AAGAAGAGGCTGCTCGACGCTCAGAGAGGCAGCTTCCCCCCTTGGGTCCAACAAACCCGCGTGTGACG
CTGGCCCCACCGTGGAATGGCCTGGCCCCCCCAGCCCCACCACCCCCACCCCGGCTGCAGATTACGGA
GAACGGCGAGTTCCGAAACACCGCAGACCACTAGCCCACCCAGCATCAGAGACCTTCTCTTCCTTTCCT
GTGCACCCCACCCTGTAACAGCACCAACCACCAGGATTGGACATCACCGAGGAACAGCGGGATTGCCT
CCCCGAATGCCTCCCTGGGAGGCACACTGATTGCCCACCCCCACCACTGCACCATTTCCAGGAGGGAG
AGTGGGGACCCTCAGCCGCCCCCTTTTCCTTCCCATTGGGGTGCTGCCCTCTCTTTGACCCCCAGGGAC
CCTTGCCCCAGGACACCGCCTACCCCGTACAGACCCCTTCACTCCGGGGTGCTATCCCCATCCTCTGCC
TCATCGTTCCCCTGAGCACTGGGGGACAGACCCTCACCCCCACCCTGGGGGTGTGGCACCTCCAAACT
TTCAACTTCAGGGTGATTTTTTTAGCAGTAACCAGAGCTGACAATCTAACTCCCCTCCACCGCCCCATTT
TGGCCTCCCCTGCCCCCCTTGTTATGGGGAGGGGACCCCGGGTGAGGGGGCCCTATTACCCCTTGATT
TCTCAGGAGCGTCTGGGGGGGCTCAGCACGCACAAACTCCTTCTCCTTCTACCACTCTTAAATTTACTC
CCTCCCCACCCAGAACCCAGATGGGGTGGAGGGGGCCACCGGGGCAGGGAGGGGGCGGCAAGGGG
GGAATGGGAGTTGTCTCCCCTTCTCCCCACACCTGATCTGCTCTCGGCTGGTCCCAGAGCGGGGTGAG
GGGGCTTATGCCCCCCCCTCCCCCAGTGTGTTGGGTGGGGTGGAATTGAGGTTAGGGTGAGGGGTCA
GGGTTTAGGAGGGTGTGTATGTTGGGAGGACAGGCTAGTTGATCTGTCCTACTCTGACACACAGTCCC
CTCTGCCCCTTCCTTCTCTCTTCTTGGTCTCTACTCCCAGGGGGAGGGGGGAACTTACTCTAGGAAAAG
CCATGTCTCTCTCCCCCAGGGTGGGGGGACCTGTGTTGGAGGAGGGGTGTTGGGGGGCCCCCTTCCA
TGACTCTGTCCCCTGGGGGAGGTAGGACAGGGCTGGGCTTCCCTCTCATCCTCCCCCTCCCAATCTCCT
TCCACCTCCCTCCCTCCCGCCAGCTCCACGATTTTTCGGTGTTTCTCTGTACATAGTTTTCTGGCGGGAT
AGGGGAGGTAGGATGGATGGGGTTTGGGGTGGGTAGGCCATGGGAGGGGAGAAGCCCCTCCTTGGC
ACCCCCTCTTCCCTGACTGCTGTCCCCTACCCAGCCTTGCCCCCTTCATCCTTTTGCGTTTGGTATTGAG
ACTCTCCTAGACTCTACTCCTCTTTCTTTTGTATGGACAGTTCCCCTTCAGTCCCATCCCCCTACACATAC
ACCCAGCCGGGGCCAAATTTATACTTATATAAAAGTTGTAAATATGTGAAATTTTATCCCTGTGCCCTTT
CCCCACCTCAGGCCCTACCCCTGGACCCTCCCCAACCTTCCTTCTCTCTTCTTTGGCTGTTGTAATTATC
TGGGGTTTGTACTGTACATATCCGGGGTGTGTGTGTGTGGGCTGGGGGCAACCCTTCTGTACAGAGCT
TCCTGGCCCCCTCCCCCCCCGCCCCTCTGCTTCCCTCCCCACCCACCACCTCAAGGGTAGGGAGTTGCT
CTTCCTACCTGTTTTATTTTGTTTTCTCGTTCTCCCTCCCCACCCCACTCCCAGCCTTATCTATCCCCCCT
CACTGTCCCCTTTTCTCCACTCCCAGCCCCATTTCCTTTTTTTCTGGAGTGTGTGGTGAAACAGAAAAAA
ACATGTTTAATAAACGGAGATTGTTCTTTTA
SEQ ID NO: 3; SynGAP1 isoform 1 (NP_006763.2):
MSRSRASIHRGSIPAMSYAPFRDVRGPSMHRTQYVHSPYDRPGWNPRFCIISGNQLLMLDEDEIHPLLIRD
RRSESSRNKLLRRTVSVPVEGRPHGEHEYHLGRSRRKSVPGGKQYSMEGAPAAPFRPSQGFLSRRLKSSIK
RTKSQPKLDRTSSFRQILPRFRSADHDRARLMQSFKESHSHESLLSPSSAAEALELNLDEDSIIKPVHSSIL
GQEFCFEVTTSSGTKCFACRSAAERDKWIENLQRAVKPNKDNSRRVDNVLKLWIIEARELPPKKRYYCELC
LDDMLYARTTSKPRSASGDTVFWGEHFEFNNLPAVRALRLHLYRDSDKKRKKDKAGYVGLVTVPVATLAG
RHFTEQWYPVTLPTGSGGSGGMGSGGGGGSGGGSGGKGKGGCPAVRLKARYQTMSILPMELYKEFAEYV
TNHYRMLCAVLEPALNVKGKEEVASALVHILQSTGKAKDFLSDMAMSEVDRFMEREHLIFRENTLATKAIEE
YMRLIGQKYLKDAIGEFIRALYESEENCEVDPIKCTASSLAEHQANLRMCCELALCKVVNSHCVFPRELKEVF
ASWRLRCAERGREDIADRLISASLFLRFLCPAIMSPSLFGLMQEYPDEQTSRTLTLIAKVIQNLANFSKFTSK
EDFLGFMNEFLELEWGSMQQFLYEISNLDTLTNSSSFEGYIDLGRELSTLHALLWEVLPQLSKEALLKLGPLP
RLLNDISTALRNPNIQRQPSRQSERPRPQPVVLRGPSAEMQGYMMRDLNSSIDLQSFMARGLNSSMDMAR
LPSPTKEKPPPPPPGGGKDLFYVSRPPLARSSPAYCTSSSDITEPEQKMLSVNKSVSMLDLQGDGPGGRLN
SSSVSNLAAVGDLLHSSQASLTAALGLRPAPAGRLSQGSGSSITAAGMRLSQMGVTTDGVPAQQLRIPLSF
QNPLFHMAADGPGPPGGHGGGGGHGPPSSHHHHHHHHHHRGGEPPGDTFAPFHGYSKSEDLSSGVPKP
PAASILHSHSYSDEFGPSGTDFTRRQLSLQDNLQHMLSPPQITIGPQRPAPSGPGGGSGGGSGGGGGGQP
PPLQRGKSQQLTVSAAQKPRPSSGNLLQSPEPSYGPARPRQQSLSKEGSIGGSGGSGGGGGGGLKPSITK
QHSQTPSTLNPTMPASERTVAWVSNMPHLSADIESAHIEREEYKLKEYSKSMDESRLDRVKEYEEEIHSLK
ERLHMSNRKLEEYERRLLSQEEQTSKILMQYQARLEQSEKRLRQQQAEKDSQIKSIIGRLMLVEEELRRDHP
AMAEPLPEPKKRLLDAQERQLPPLGPTNPRVTLAPPWNGLAPPAPPPPPRLQITENGEFRNTADH
SEQ ID NO: 4; SynGAP1 isoform 2 (NP_001123538.1):
MSRSRASIHRGSIPAMSYAPFRDVRGPSMHRTQYVHSPYDRPGWNPRFCIISGNQLLMLDEDEIHPLLIRD
RRSESSRNKLLRRTVSVPVEGRPHGEHEYHLGRSRRKSVPGGKQYSMEGAPAAPFRPSQGFLSRRLKSSIK
RTKSQPKLDRTSSFRQILPRFRSADHDRARLMQSFKESHSHESLLSPSSAAEALELNLDEDSIIKPVHSSIL
GQEFCFEVTTSSGTKCFACRSAAERDKWIENLQRAVKPNKDNSRRVDNVLKLWIIEARELPPKKRYYCELC
LDDMLYARTTSKPRSASGDTVFWGEHFEFNNLPAVRALRLHLYRDSDKKRKKDKAGYVGLVTVPVATLAG
RHFTEQWYPVTLPTGSGGSGGMGSGGGGGSGGGSGGKGKGGCPAVRLKARYQTMSILPMELYKEFAEYV
TNHYRMLCAVLEPALNVKGKEEVASALVHILQSTGKAKDFLSDMAMSEVDRFMEREHLIFRENTLATKAIEE
YMRLIGQKYLKDAIGEFIRALYESEENCEVDPIKCTASSLAEHQANLRMCCELALCKVVNSHCVFPRELKEVF
ASWRLRCAERGREDIADRLISASLFLRFLCPAIMSPSLFGLMQEYPDEQTSRTLTLIAKVIQNLANFSKFTSK
EDFLGFMNEFLELEWGSMQQFLYEISNLDTLTNSSSFEGYIDLGRELSTLHALLWEVLPQLSKEALLKLGPLP
RLLNDISTALRNPNIQRQPSRQSERPRPQPVVLRGPSAEMQGYMMRDLNSSMDMARLPSPTKEKPPPPPPG
GGKDLFYVSRPPLARSSPAYCTSSSDITEPEQKMLSVNKSVSMLDLQGDGPGGRLNSSSVSNLAAVGDLL
HSSQASLTAALGLRPAPAGRLSQGSGSSITAAGMRLSQMGVTTDGVPAQQLRIPLSFQNPLFHMAADGPG
PPGGHGGGGGHGPPSSHHHHHHHHHHRGGEPPGDTFAPFHGYSKSEDLSSGVPKPPAASILHSHSYSDE
FGPSGTDFTRRQLSLQDNLQHMLSPPQITIGPQRPAPSGPGGGSGGGSGGGGGGQPPPLQRGKSQQLTV
SAAQKPRPSSGNLLQSPEPSYGPARPRQQSLSKEGSIGGSGGSGGGGGGGLKPSITKQHSQTPSTLNPTM
PASERTVAWVSNMPHLSADIESAHIEREEYKLKEYSKSMDESRLDREYEEEIHSLKERLHMSNRKLEEYERR
LLSQEEQTSKILMQYQARLEQSEKRLRQQQAEKDSQIKSIIGRLMLVEEELRRDHPAMAEPLPEPKKRLLDA
QRGSFPPWVQQTRV

1. A method for increasing levels of SynGAP1 protein in a cell, comprising contacting the cell with an antisense oligonucleotide that enhances splicing at a splice site of a retained intron in an intron-retaining SynGAP1 mRNA or pre-mRNA, wherein the retained intron is selected from among intron 5, 8, 9, 12, 13 and 14 and wherein the antisense oligonucleotide comprises a sequence of nucleobases that is complementary to a target region in the SynGAP1 mRNA or pre-mRNA.

2-61. (canceled)
